SEQUENCE LISTING (17 sequences)

SEQ ID NO: 1 - DNA, 1608 bp, synthetic construct
  source 1..1608: mol_type = other DNA
atggcgtccctttcccttgcacctgttaacatctttaaggcaggagctgatgaagagagagcagagacagctcgtctgacttcttttattggtgccatcgccattggagacttggtaaagagcaccttgggacccaaaggcatggacaaaattcttctaagcagtggacgagatgcctctcttatggtaaccaatgatggtgccactattctaaaaaacattggtgttgacaatccagcagctaaagttttagttgatatgtcaagggttcaagatgatgaagttggtgatggcactacctctgttaccgttttagcagcagaattattaagggaagcagaatctttaattgcaaaaaagattcatccacagaccatcatagcgggttggagagaagccacgaaggctgcaagagaggcgctgttgagttctgcagttgatcatggttccgatgaagttaaattccgtcaagatttaatgaatattgcgggcacaacattatcctcaaaacttcttactcatcacaaagaccactttacaaagttagctgtagaagcagttctcagactgaaaggctctggcaacctggaggcaattcatattatcaagaagctaggaggaagtttggcagattcctatttagatgaaggcttcctgttggataaaaaaattggagtaaatcaaccaaaacgaattgaaaatgctaaaattcttattgcaaatactggtatggatacagacaaaataaagatatttggttcccgggtaagagttgactctacagcaaaggttgcagaaatagaacatgcggaaaaggaaaaaatgaaggagaaagttgaacgtattcttaagcatggaataaattgctttattaacaggcaattaatttataattatcctgaacagctctttggtgctgctggtgtcatggctattgagcatgcagattttgcaggtgtggaacgcctagctcttgtcacaggtggtgaaattgcctctacctttgatcacccagaactggtgaagcttggaagttgcaaacttatcgaggaagtcatgattggagaagacaaactcattcacttttctggggttgcccttggtgaggcttgtaccattgttttgcgtggtgccactcaacaaattttagatgaagcagaaagatcattgcatgatgctctttgtgttcttgcgcaaactgtaaaggactctagaacagtttatggaggaggctgttctgagatgttgatggctcatgctgtgacacagcttgccaatagaacaccaggcaaagaagctgttgcaatggagtcttatgctaaagcactgagaatgttgccaaccatcatagctgacaatgcaggctatgacagtgcagacctggtggcacagctcagggctgctcacagtgaaggcaataccactgctggattggatatgagggaaggcaccattggagatatggctatcctgggtataacagaaagttttcaagtgaagcgacaggttcttctgagtgcagctgaagcagcagaggtgattctgcgtgtggacaacatcatcaaagcggcacccaggaaacgtgtccctgatcaccacccctgttaa

SEQ ID NO: 2 - DNA, 1596 bp, synthetic construct
  source 1..1596: mol_type = other DNA
atggcggcggtgaagaccctgaaccccaaggccgaggtggcccgagcgcaggcggcgctggcggtcaacatcagcgcagcgcggggtctgcaggacgtgctaaggaccaacctggggcccaagggcaccatgaagatgctcgtttctggcgctggagacatcaaacttactaaagacggcaatgtgctgcttcacgaaatgcaaattcaacacccaacagcttccttaatagcaaaggtagcaacagcccaggatgatataactggtgatggtacgacttctaatgtcctaatcattggagagctgctgaaacaggcggatctctacatttctgaaggccttcatcctagaataatcactgaaggatttgaagctgcaaaggaaaaggcccttcagtttttggaagaagtcaaagtaagcagagagatggacagggaaacacttatagatgtggccagaacatctcttcgtactaaagttcatgctgaacttgcagatgtcttaacagaggctgtagtggactccattttggccattaaaaagcaagatgaacctattgatctcttcatgattgagatcatggagatgaaacataaatctgaaactgatacaagcttaatcagagggcttgttttggaccacggagcacggcatcctgatatgaagaaaagggtggaggatgcatacatcctcacttgtaacgtgtcattagagtatgagaaaacagaagtgaattctggctttttttacaagagtgcagaagagagagaaaaactcgtgaaagctgaaagaaaattcattgaagatagggttaaaaaaataatagaactgaaaaggaaagtctgtggcgattcagataaaggatttgttgttattaatcaaaagggaattgaccccttttccttagatgctctttcaaaagaaggcatagttgctctgcgcagagctaaaaggagaaatatggagaggctgactcttgcttgtggtggggtagccctgaattcttttgacgacctaagtcctgactgcttgggacatgcaggacttgtatatgagtatacattgggagaagagaagtttacctttattgagaaatgtaacaaccctcgttctgtcacattattgatcaaaggaccaaataagcacacactcactcagatcaaagatgcagtgagggacggcttgagggctgtcaaaaatgctattgatgatggctgtgtggttccaggtgctggtgccgtggaagtggcaatggcagaagccctgattaaacataagcccagtgtaaagggcagggcacagcttggagtccaagcatttgctgatgcattgctcattattcccaaggttcttgctcagaactctggttttgaccttcaggaaacattagttaaaattcaagcagaacattcagaatcaggtcagcttgtgggtgtggacctgaacacaggtgagccaatggtggcagcagaagtaggcgtatgggataactattgtgtaaagaaacagcttcttcactcctgcactgtgattgccaccaacattctcttggttgatgagatcatgcgagctggaatgtcttctctgaaaggttga

SEQ ID NO: 3 - DNA, 1671 bp, synthetic construct
  source 1..1671: mol_type = other DNA
atggaggggcctttgtccgtgttcggtgaccgcagcactggggaaacgatccgctcccaaaacgttatggctgcagcttcgattgccaatattgtaaaaagttctcttggtccagttggcttggataaaatgttggtggatgatattggtgatgtaaccattactaacgatggtgcaaccatcctgaagttactggaggtagaacatcctgcagctaaagttctttgtgagctggctgatctgcaagacaaagaagttggagatggaactacttcagtggttattattgcagcagaactcctaaaaaatgcagatgaattagtcaaacagaaaattcatcccacatcagttattagtggctatcgacttgcttgcaaggaagcagtgcgttatatcaatgaaaacctaattgttaacacagatgaactgggaagagattgcctgattaatgctgctaagacatccatgtcttccaaaatcattggaataaatggtgatttctttgctaacatggtagtagatgctgtacttgctattaaatacacagacataagaggccagccacgctatccagtcaactctgttaatattttgaaagcccatgggagaagtcaaatggagagtatgctcatcagtggctatgcactcaactgtgtggtgggatcccagggcatgcccaagagaatcgtaaatgcaaaaattgcttgccttgacttcagcctgcaaaaaacaaaaatgaagcttggtgtacaggtggtcattacagaccctgaaaaactggaccaaattagacagagagaatcagatatcaccaaggagagaattcagaagatcctggcaactggtgccaatgttattctaaccactggtggaattgatgatatgtgtctgaagtattttgtggaggctggtgctatggcagttagaagagttttaaaaagggaccttaaacgcattgccaaagcttctggagcaactattctgtcaaccctggccaatttggaaggtgaagaaacttttgaagctgcaatgttgggacaggcagaagaagtggtacaggagagaatttgtgatgatgagctgatcttaatcaaaaatactaaggctcgtacgtctgcatcgattatcttacgtggggcaaatgatttcatgtgtgatgagatggagcgctctttacatgatgcactttgtgtagtgaagagagttttggagtcaaaatctgtggttcccggtgggggtgctgtagaagcagccctttccatataccttgaaaactatgcaaccagcatggggtctcgggaacagcttgcgattgcagagtttgcaagatcacttcttgttattcccaatacactagcagttaatgctgcccaggactccacagatctggttgcaaaattaagagcttttcataatgaggcccaggttaacccagaacgtaaaaatctaaaatggattggtcttgatttgagcaatggtaaacctcgagacaacaaacaagcaggggtgtttgaaccaaccatagttaaagttaagagtttgaaatttgcaacagaagctgcaatcaccattcttcgaattgatgatcttattaaattacatccagaaagtaaagatgataaacatggaagttatgaagatgctgttcactctggagcccttaatgattga

SEQ ID NO: 4 - DNA, 1638 bp, synthetic construct
  source 1..1638: mol_type = other DNA
atgatgggccatcgtccagtgctcgtgctcagccagaacacaaagcgtgaatccggaagaaaagttcaatctggaaacatcaatgctgccaagactattgcagatatcatccgaacatgtttgggacccaagtccatgatgaagatgcttttggacccaatgggaggcattgtgatgaccaatgatggcaatgccattcttcgagagattcaagtccagcatccagcggccaagtccatgatcgaaattagccggacccaggatgaagaggttggagatgggaccacatcagtaattattcttgcaggggaaatgctgtctgtagctgagcacttcctggagcagcagatgcacccaacagtggtgatcagtgcttaccgcaaggcattggatgatatgatcagcaccctaaagaaaataagtatcccagtcgacatcagtgacagtgatatgatgctgaacatcatcaacagctctattactaccaaagccatcagtcggtggtcatctttggcttgcaacattgccctggatgctgtcaagatggtacagtttgaggagaatggtcggaaagagattgacataaaaaaatatgcaagagtggaaaagatacctggaggcatcattgaagactcctgtgtcttgcgtggagtcatgattaacaaggatgtgacccatccacgtatgcggcgctatatcaagaaccctcgcattgtgctgctggattcttctctggaatacaagaaaggagaaagccagactgacattgagattacacgagaggaggacttcacccgaattctccagatggaggaagagtacatccagcagctctgtgaggacattatccaactgaagcccgatgtggtcatcactgaaaagggcatctcagatttagctcagcactaccttatgcgggccaatatcacagccatccgcagagtccggaagacagacaataatcgcattgctagagcctgtggggcccggatagtcagccgaccagaggaactgagagaagatgatgttggaacaggagcaggcctgttggaaatcaagaaaattggagatgaatactttactttcatcactgactgcaaagaccccaaggcctgcaccattctcctccggggggctagcaaagagattctctcggaagtagaacgcaacctccaggatgccatgcaagtgtgtcgcaatgttctcctggaccctcagctggtgccagggggtggggcctccgagatggctgtggcccatgccttgacagaaaaatccaaggccatgactggtgtggaacaatggccatacagggctgttgcccaggccctagaggtcattcctcgtaccctgatccagaactgtggggccagcaccatccgtctacttacctcccttcgggccaagcacacccaggagaactgtgagacctggggtgtaaatggtgagacgggtactttggtggacatgaaggaactgggcatatgggagccattggctgtgaagctgcagacttataagacagcagtggagacggcagttctgctactgcgaattgatgacatcgtttcaggccacaaaaagaaaggcgatgaccagagccggcaaggcggggctcctgatgctggccaggagtga

SEQ ID NO: 5 - DNA, 2040 bp, synthetic construct
  source 1..2040: mol_type = other DNA
atgataagtgccagccgagctgcagcagcccgtctcgtgggcgccgcagcctcccggggccctacggccgcccgccaccaggatagctggaatggccttagtcatgaggcttttagacttgtttcaaggcgggattatgcatcagaagcaatcaagggagcagttgttggtattgatttgggtactaccaactcctgcgtggcagttatggaaggtaaacaagcaaaggtgctggagaatgccgaaggtgccagaaccaccccttcagttgtggcctttacagcagatggtgagcgacttgttggaatgccggccaagcgacaggctgtcaccaacccaaacaatacattttatgctaccaagcgtctcattggccggcgatatgatgatcctgaagtacagaaagacattaaaaatgttccctttaaaattgtccgtgcctccaatggtgatgcctgggttgaggctcatgggaaattgtattctccgagtcagattggagcatttgtgttgatgaagatgaaagagactgcagaaaattacttggggcacacagcaaaaaatgctgtgatcacagtcccagcttatttcaatgactcgcagagacaggccactaaagatgctggccagatatctggactgaatgtgcttcgggtgattaatgagcccacagctgctgctcttgcctatggtctagacaaatcagaagacaaagtcattgctgtatatgatttaggtggtggaacttttgatatttctatcctggaaattcagaaaggagtatttgaggtgaaatccacaaatggggataccttcttaggtggggaagactttgaccaggccttgctacggcacattgtgaaggagttcaagagagagacaggggttgatttgactaaagacaacatggcacttcagagggtacgggaagctgctgaaaaggctaaatgtgaactctcctcatctgtgcagactgacatcaatttgccctatcttacaatggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcattgaagctgttcgaaatggcatacaaaaagatggcatctgagcgagaaggctctggaagttctggcactggggaacaaaaggaagatcaaaaggaggaaaaacagtaa

SEQ ID NO: 6 - DNA, 2175 bp, synthetic construct
  source 1..2175: mol_type = other DNA
atgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattag

SEQ ID NO: 7 - protein, 535 aa, synthetic construct
MASLSLAPVNIFKAGADEERAETARLTSFIGAIAIGDLVKSTLGPKGMDKILLSSGRDASLMVTNDGATILKNIGVDNPAAKVLVDMSRVQDDEVGDGTTSVTVLAAELLREAESLIAKKIHPQTIIAGWREATKAAREALLSSAVDHGSDEVKFRQDLMNIAGTTLSSKLLTHHKDHFTKLAVEAVLRLKGSGNLEAIHIIKKLGGSLADSYLDEGFLLDKKIGVNQPKRIENAKILIANTGMDTDKIKIFGSRVRVDSTAKVAEIEHAEKEKMKEKVERILKHGINCFINRQLIYNYPEQLFGAAGVMAIEHADFAGVERLALVTGGEIASTFDHPELVKLGSCKLIEEVMIGEDKLIHFSGVALGEACTIVLRGATQQILDEAERSLHDALCVLAQTVKDSRTVYGGGCSEMLMAHAVTQLANRTPGKEAVAMESYAKALRMLPTIIADNAGYDSADLVAQLRAAHSEGNTTAGLDMREGTIGDMAILGITESFQVKRQVLLSAAEAAEVILRVDNIIKAAPRKRVPDHHPC

SEQ ID NO: 8 - protein, 531 aa, synthetic construct
MAAVKTLNPKAEVARAQAALAVNISAARGLQDVLRTNLGPKGTMKMLVSGAGDIKLTKDGNVLLHEMQIQHPTASLIAKVATAQDDITGDGTTSNVLIIGELLKQADLYISEGLHPRIITEGFEAAKEKALQFLEEVKVSREMDRETLIDVARTSLRTKVHAELADVLTEAVVDSILAIKKQDEPIDLFMIEIMEMKHKSETDTSLIRGLVLDHGARHPDMKKRVEDAYILTCNVSLEYEKTEVNSGFFYKSAEEREKLVKAERKFIEDRVKKIIELKRKVCGDSDKGFVVINQKGIDPFSLDALSKEGIVALRRAKRRNMERLTLACGGVALNSFDDLSPDCLGHAGLVYEYTLGEEKFTFIEKCNNPRSVTLLIKGPNKHTLTQIKDAVRDGLRAVKNAIDDGCVVPGAGAVEVAMAEALIKHKPSVKGRAQLGVQAFADALLIIPKVLAQNSGFDLQETLVKIQAEHSESGQLVGVDLNTGEPMVAAEVGVWDNYCVKKQLLHSCTVIATNILLVDEIMRAGMSSLKG

SEQ ID NO: 9 - protein, 556 aa, synthetic construct
MEGPLSVFGDRSTGETIRSQNVMAAASIANIVKSSLGPVGLDKMLVDDIGDVTITNDGATILKLLEVEHPAAKVLCELADLQDKEVGDGTTSVVIIAAELLKNADELVKQKIHPTSVISGYRLACKEAVRYINENLIVNTDELGRDCLINAAKTSMSSKIIGINGDFFANMVVDAVLAIKYTDIRGQPRYPVNSVNILKAHGRSQMESMLISGYALNCVVGSQGMPKRIVNAKIACLDFSLQKTKMKLGVQVVITDPEKLDQIRQRESDITKERIQKILATGANVILTTGGIDDMCLKYFVEAGAMAVRRVLKRDLKRIAKASGATILSTLANLEGEETFEAAMLGQAEEVVQERICDDELILIKNTKARTSASIILRGANDFMCDEMERSLHDALCVVKRVLESKSVVPGGGAVEAALSIYLENYATSMGSREQLAIAEFARSLLVIPNTLAVNAAQDSTDLVAKLRAFHNEAQVNPERKNLKWIGLDLSNGKPRDNKQAGVFEPTIVKVKSLKFATEAAITILRIDDLIKLHPESKDDKHGSYEDAVHSGALND

SEQ ID NO: 10 - protein, 545 aa, synthetic construct
MMGHRPVLVLSQNTKRESGRKVQSGNINAAKTIADIIRTCLGPKSMMKMLLDPMGGIVMTNDGNAILREIQVQHPAAKSMIEISRTQDEEVGDGTTSVIILAGEMLSVAEHFLEQQMHPTVVISAYRKALDDMISTLKKISIPVDISDSDMMLNIINSSITTKAISRWSSLACNIALDAVKMVQFEENGRKEIDIKKYARVEKIPGGIIEDSCVLRGVMINKDVTHPRMRRYIKNPRIVLLDSSLEYKKGESQTDIEITREEDFTRILQMEEEYIQQLCEDIIQLKPDVVITEKGISDLAQHYLMRANITAIRRVRKTDNNRIARACGARIVSRPEELREDDVGTGAGLLEIKKIGDEYFTFITDCKDPKACTILLRGASKEILSEVERNLQDAMQVCRNVLLDPQLVPGGGASEMAVAHALTEKSKAMTGVEQWPYRAVAQALEVIPRTLIQNCGASTIRLLTSLRAKHTQENCETWGVNGETGTLVDMKELGIWEPLAVKLQTYKTAVETAVLLLRIDDIVSGHKKKGDDQSRQGGAPDAGQE

SEQ ID NO: 11 - protein, 679 aa, synthetic construct
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 12 - protein, 724 aa, synthetic construct
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 13 - protein, 334 aa, synthetic construct
MGFLLDKKIGVNQPKRIENAKILIANTGMDTDKIKIFGSRVRVDSTAKVAEIEHAEKEKMKEKVERILKHGINCFINRQLIYNYPEQLFGAAGVMAIEHADFAGVERLALVTGGEIASTFDHPELVKLGSCKLIEEVMIGEDKLIHFSGVALGGKPIPNPLLGLDSTEACTIVLRGATQQILDEAERSLHDALCVLAQTVKDSRTVYGGGCSEMLMAHAVTQLANRTPGKEAVAMESYAKALRMLPTIIADNAGYDSADLVAQLRAAHSEGNTTAGLDMREGTIGDMAILGITESFQVKRQVLLSAAEAAEVILRVDNIIKAAPRKRVPDHHPC

SEQ ID NO: 14 - protein, 187 aa, synthetic construct
MGFLLDKKIGVNQPKRIENAKILIANTGMDTDKIKIFGSRVRVDSTAKVAEIEHAEKEKMKEKVERILKHGINCFINRQLIYNYPEQLFGAAGVMAIEHADFAGVERLALVTGGEIASTFDHPELVKLGSCKLIEEVMIGEDKLIHFSGVALGGSGGILGITESFQVKRQVLLSAAEAAEVILRVDN

SEQ ID NO: 15 - DNA, 1005 bp, synthetic construct
  source 1..1005: mol_type = other DNA
atgggcttcctgttggataaaaaaattggagtaaatcaaccaaaacgaattgaaaatgctaaaattcttattgcaaatactggtatggatacagacaaaataaagatatttggttcccgggtaagagttgactctacagcaaaggttgcagaaatagaacatgcggaaaaggaaaaaatgaaggagaaagttgaacgtattcttaagcatggaataaattgctttattaacaggcaattaatttataattatcctgaacagctctttggtgctgctggtgtcatggctattgagcatgcagattttgcaggtgtggaacgcctagctcttgtcacaggtggtgaaattgcctctacctttgatcacccagaactggtgaagcttggaagttgcaaacttatcgaggaagtcatgattggagaagacaaactcattcacttttctggggttgcccttggtggtaagcctatccctaaccctctcctcggtctcgattctacggaggcttgtaccattgttttgcgtggtgccactcaacaaattttagatgaagcagaaagatcattgcatgatgctctttgtgttcttgcgcaaactgtaaaggactctagaacagtttatggaggaggctgttctgagatgttgatggctcatgctgtgacacagcttgccaatagaacaccaggcaaagaagctgttgcaatggagtcttatgctaaagcactgagaatgttgccaaccatcatagctgacaatgcaggctatgacagtgcagacctggtggcacagctcagggctgctcacagtgaaggcaataccactgctggattggatatgagggaaggcaccattggagatatggctatcctgggtataacagaaagttttcaagtgaagcgacaggttcttctgagtgcagctgaagcagcagaggtgattctgcgtgtggacaacatcatcaaagcggcacccaggaaacgtgtccctgatcaccacccctgttag

SEQ ID NO: 16 - DNA, 564 bp, synthetic construct
  source 1..564: mol_type = other DNA
atgggcttcctgttggataaaaaaattggagtaaatcaaccaaaacgaattgaaaatgctaaaattcttattgcaaatactggtatggatacagacaaaataaagatatttggttcccgggtaagagttgactctacagcaaaggttgcagaaatagaacatgcggaaaaggaaaaaatgaaggagaaagttgaacgtattcttaagcatggaataaattgctttattaacaggcaattaatttataattatcctgaacagctctttggtgctgctggtgtcatggctattgagcatgcagattttgcaggtgtggaacgcctagctcttgtcacaggtggtgaaattgcctctacctttgatcacccagaactggtgaagcttggaagttgcaaacttatcgaggaagtcatgattggagaagacaaactcattcacttttctggggttgcccttggtggaagtggtggaatcctgggtataacagaaagttttcaagtgaagcgacaggttcttctgagtgcagctgaagcagcagaggtgattctgcgtgtggacaactga

SEQ ID NO: 17 - protein, 14 aa, synthetic construct
GKPIPNPLLGLDST

(19) United States
(12) Patent Application Publication
(10) Pub. No.: US 2024/0350586 A1
(43) Pub. Date: Oct. 24, 2024

(54) CHAPERONES AS AN AUTOPHAGY RECEPTORS FOR CLEARANCES OF PROTEIN AGGREGATES AND/OR AGGREGATION-PRONE PROTEINS

(71) Applicant: TSINGHUA UNIVERSITY, Beijing (CN)

(72) Inventors: Liang GE, Beijing (CN); Min ZHANG, Beijing (CN); Xinyu MA, Beijing (CN); Caijing LU, Beijing (CN); Shulin LI, Beijing (CN); Xuan TAO, Beijing (CN)

(21) Appl. No.: 18/761,349

(22) Filed: Jul. 2, 2024

Related U.S. Application Data
(63) Continuation of application No. PCT/CN2022/082587, filed on Mar. 23, 2022.

(30) Foreign Application Priority Data
Jan. 5, 2022 (WO) PCT/CN2022/070356

(51) Int. Cl. A61K 38/17; A61K 48/00; A61K 49/00; A61P 25/28; G01N 33/50

(52) U.S. Cl. CPC A61K 38/1709; A61K 48/0033; A61K 49/0008; A61P 25/28; G01N 33/5058

(57) ABSTRACT

Use of chaperones as autophagy receptors. The inventors identify a new function of the chaperones in aggrephagy. The chaperones are as a new type of autophagy receptor regulating the clearance of aggregation-prone proteins in cell and mouse brain.

### CROSS-REFERENCE TO RELATED APPLICATIONS

This application is a continuation of International Application No. PCT/CN2022/082587, filed on Mar. 23, 2022, which claims priority to International Application No. PCT/CN2022/070356, filed on Jan. 5, 2022. The disclosures of the aforementioned applications are hereby incorporated by reference in their entireties.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence_Listing.xml”, was created on Jun. 12, 2024, and is approximately 32 kilobytes in size.

### FIELD

The present invention relates to biotechnology, especially to chaperones as autophagy receptors, more especially to use of CCT2 as an autophagy receptor, a method for promoting clearance of solid protein aggregates and/or aggregation-prone proteins, a method for promoting autophagosomes targeting to inclusion bodies, use of reagents in the preparation of drugs for the treatment or prevention of diseases caused by protein aggregation, a method for treating or preventing of diseases caused by protein aggregation and a method for screening drugs for treatment or prevention diseases caused by protein aggregation.

### BACKGROUND

Accumulation of protein aggregates is a hallmark of multiple human pathologies, including neurodegeneration, eye disease, and type II diabetes. Sufficient amounts of evidence demonstrate autophagy, a lysosome-mediated bulk degradation pathway, as a key cellular process to clear protein aggregates. How the autophagic membrane recognizes protein aggregates and selectively targets them for degradation has been a major question in the field. Ubiquitin-binding receptors (P62 (SQSTM1), NBR1, TAXIBP1, Optineurin, and Tollip) can mediate but not specific for the autophagic clearance of protein aggregates, which may decrease the effects or increase the risk of side effects when using these receptors as therapeutic target. Therefore, it's urgent to find out a new type of autophagy receptor which specifically recognize and degrade protein aggregates. It has been unclear whether specific aggrephagy receptors exist in mammals and, if they exist, how they regulate aggrephagy.

It has been shown that aggregation-prone proteins form phase-separated biomolecular condensates/droplets before transitioning into pathogenic solid protein aggregates. Autophagy has been proposed to preferentially clear protein condensates with a certain amount of liquidity using the known aggrephagy receptors Atg19 (S. cerevisiae) or SEPA-1 (C. elegans). In addition, the ubiquitin-binding receptors, P62 and NBR1, organize the formation of phase-separated condensates, which likely facilitates autophagic clearance of the protein condensates. However, it remains to clarify how pathogenic solid protein aggregates are recognized and selectively cleared by aggrephagy.

In the cell, proteostasis is tightly controlled by a network of molecular chaperones which maintain protein folding and cooperates with the degradation machinery. Chaperones have been shown to be upregulated in response to misfolded protein accumulation to counteract aberrant folding and aggregation via direct binding to the misfolded protein. In the past ˜20 years, it has been shown by multiple groups that multiple chaperones become major components of the protein aggregate when aggregation occurs. However, it is by far, unknown about the function of the aggregate-associated chaperones.

### SUMMARY

The aim of the present invention is to solve at least one of the technical problems of the prior art. The present invention is based on the following findings of the present inventor:

In the current work, the inventors identify a new function of the TRIC subunit CCT2 in aggrephagy, which is conserved in mammals and yeast. CCT2 promotes autophagosome incorporation and clearance of protein aggregates with little liquidity via interacting with ATG8s and aggregation-prone proteins independent of cargo ubiquitination. In addition, CCT2 acts independently of the known lysosome-mediated pathways for clearance of aggregation-prone proteins, including ubiquitin-binding receptors (P62, NBR1, and TAX1BP1)-mediated aggrephagy and chaperone-mediated autophagy (CMA). CCT2 switches its function from a chaperone to an autophagy receptor via monomer formation, which exposes its ATG8-interaction motif and therefore allows for the recruitment of autophagosomal membranes. The dual function of CCT2, as a chaperone and an aggrephagy receptor, enables double-layer maintenance of proteostasis. In addition, the inventors also identified other chaperones, including CCT6, CCT1, CCT3, HSPA9, and HSP90AB1, which can also promote degradation of aggregation-prone proteins. Of the five chaperones, CCT6, CCT1, CCT3 and HSPA9 can also associate with ATG8s and enhance autophagosomal membrane targeting to protein aggregates.

As a result, the present disclosure provides use of chaperone as an autophagy receptor.

According to an embodiment of the present invention, the chaperone comprises at least one of the following: CCT2, CCT6, CCT1, CCT3, HSPA9 and HSP90AB1.

The inventors identify a new function of the chaperones in aggrephagy. The chaperonin subunit, such as CCT2, CCT6, CCT1, CCT3, HSPA9 or HSP90AB1 is as a new autophagy receptor regulating the clearance of aggregation-prone proteins in cell and mouse brain.

In one aspect of present disclosure, a method for promoting clearance of solid protein aggregates and/or aggregation-prone proteins is provided. According to an embodiment of the present invention, the method comprises giving reagents, which are used to at least one of the following: overexpress chaperones or enhance the activity of chaperones; enhance the chaperones interaction with solid protein aggregates and/or aggregation-prone proteins; enhance the chaperones interaction with ATG8s; promote the disassociation of TRIC to produce free subunits; overexpress/apply the D2 and/or D3 domain of CCT2 or enhance the D2 and/or D3 domain activity of CCT2; overexpress/apply the P7 Peptide of CCT2 or enhance the P7 Peptide activity of CCT2; enhance the activity of amino acids 503 ˜505 and/or 513 ˜515 of CCT2; overexpress/apply the peptide or enhance the peptide activity, wherein the peptide comprises amino acids 503 to 515 of CCT2 and optionally at least 10 amino acids upstream of amino acid 503 or at least 10 amino acids downstream of amino acid 515. The inventors found that the chaperones (such as CCT2, CCT6, CCT1, and CCT3) specifically promotes clearance of solid aggregates instead of liquid-granules caused by phase separation. As a representative of mechanistic study, the inventors found that the chaperone (CCT2) associates with aggregation-prone proteins independent of cargo ubiquitination and interacts with autophagosome marker ATG8s. The inventors also found that CCT2 interacts with autophagosome marker ATG8s through a non-classical VLIR motif (amino acids 503 ˜505 and/or 513 ˜515 of CCT2). The VLIR motifs are buried in the TRIC complex under steady states. Excessive aggregation-prone protein induced the formation of CCT2 monomer, exposing the VLIR motifs and enabling it to interact with ATG8 family members. The above method according to the embodiment of the invention can significantly promote the removal of solid protein aggregates and/or aggregation-prone proteins.

In one aspect of present disclosure, a method for promoting ATG8 targeting to inclusion bodies. According to an embodiment of the present invention, the method comprises: giving reagent, which is used to at least one of the following: overexpress chaperones or enhance the activity of chaperones; enhance the chaperones interaction with ATG8s; promote the disassociation of TRIC to produce free subunits; overexpress/apply the D2 and/or D3 domain of CCT2 or enhance the D2 and/or D3 domain activity of CCT2; overexpress/apply the P7 Peptide of CCT2 or enhance the P7 Peptide activity of CCT2; enhance the activity of amino acids 503 ˜505 and/or 513 ˜515 of CCT2; overexpress/apply the peptide or enhance the peptide activity, wherein the peptide comprises amino acids 503 to 515 of CCT2 and optionally at least 10 amino acids upstream of amino acid 503 or at least 10 amino acids downstream of amino acid 515. The inventors found that chaperones are as the new autophagy receptor regulating the clearance of aggregation-prone proteins, which is responsible for ATG8 targeting to inclusion bodies. The chaperones such as CCT2 interacts with autophagosome marker ATG8s through a non-classical VLIR motif (amino acids 503 ˜505 and/or 513 ˜515 of CCT2). The VLIR motifs are buried in the TRIC complex under steady states. Excessive aggregation-prone protein induced the formation of CCT2 monomer, exposing the VLIR motifs and enabling it to interact with ATG8 family members. The above method according to the embodiment of the invention can significantly promote ATG8 targeting to inclusion bodies.

According to an embodiment of the present invention, the chaperone comprises at least one of the following: CCT2, CCT6, CCT1, CCT3, HSPA9 and HSP90AB1.

According to an embodiment of the present invention, the method for promoting clearance of solid protein aggregates and/or aggregation-prone proteins comprises: giving reagents, which are used to at least one of the following: overexpress CCT2 or enhance the activity of CCT2;enhance the CCT2 interaction with solid protein aggregates and/or aggregation-prone proteins; enhance the CCT2 interaction with ATG8s; overexpress CCT6 or enhance the activity of CCT6; enhance the CCT6 interaction with solid protein aggregates and/or aggregation-prone proteins; enhance the CCT6 interaction with ATG8s; overexpress CCT1 or enhance the activity of CCT1; enhance the CCT1 interaction with solid protein aggregates and/or aggregation-prone proteins; enhance the CCT1 interaction with ATG8s; overexpress CCT3 or enhance the activity of CCT3; enhance the CCT3 interaction with solid protein aggregates and/or aggregation-prone proteins; enhance the CCT3 interaction with ATG8s; overexpress HSPA9 or enhance the activity of HSPA9; enhance the HSPA9 interaction with solid protein aggregates and/or aggregation-prone proteins; enhance the HSPA9interaction with ATG8s; overexpress HSP90AB1 or enhance the activity of HSP90AB1;enhance the HSP90AB1 interaction with solid protein aggregates and/or aggregation-prone proteins; overexpress/apply the P7 Peptide of CCT2 or enhance the P7 Peptide activity of CCT2; enhance the activity of amino acids 503 ˜505 and/or 513 ˜515 of CCT2; overexpress/apply the peptide or enhance the peptide activity, wherein the peptide in comprises amino acids 503 to 515 of CCT2 and optionally at least 10 amino acids upstream of amino acid 503 or at least 10 amino acids downstream of amino acid 515.

According to an embodiment of the present invention, the free subunits comprise at least one of the following: CCT2, CCT6, CCT1, CCT3.

According to an embodiment of the present invention, the method for promoting ATG8 targeting to inclusion bodies comprises: giving reagent, which is used to at least one of the following: overexpress CCT2 or enhance the activity of CCT2; enhance the CCT2 interaction with ATG8s; overexpress CCT6 or enhance the activity of CCT6; enhance the CCT6 interaction with ATG8s; overexpress CCT1 or enhance the activity of CCT1; enhance the CCT1 interaction with ATG8s; overexpress CCT3 or enhance the activity of CCT3; enhance the CCT3 interaction with ATG8s; overexpress HSPA9 or enhance the activity of HSPA9; enhance the HSPA9 interaction with ATG8s; overexpress chaperones or enhance the activity of HSP90AB1; overexpress/apply the P7 Peptide of CCT2 or enhance the P7 Peptide activity of CCT2; enhance the activity of amino acids 503 ˜505 and/or 513 ˜515 of CCT2; overexpress/apply the peptide or enhance the peptide activity, wherein the peptide comprises amino acids 503 to 515 of CCT2 and optionally at least 10 amino acids upstream of amino acid 503 or at least 10 amino acids downstream of amino acid 515.

According to an embodiment of the present invention, the reagent comprises expression vector with CCT2 coding nucleic acid or compounds, protein, or factors used for enhancing the activity of chaperones.

According to an embodiment of the present invention, the reagent comprises expression vector with D2 and/or D3 domain coding nucleic acid or compounds, protein or factors used for enhancing the activity of D2 and/or D3 domain.

According to an embodiment of the present invention, the CCT2 coding nucleic acid has the nucleotide sequence shown in SEQ ID No: 1; or CCT6 coding nucleic acid has the nucleotide sequence shown in SEQ ID No: 2; or CCT1 coding nucleic acid has the nucleotide sequence shown in SEQ ID No: 3; or CCT3 coding nucleic acid has the nucleotide sequence shown in SEQ ID No: 4; or HSPA9 coding nucleic acid has the nucleotide sequence shown in SEQ ID No: 5; or HSP90AB1 coding nucleic acid has the nucleotide sequence shown in SEQ ID No: 6.

(SEQ ID NO: 1)
ATGGCGTCCCTTTCCCTTGCACCTGTTAACATCTTTAAGGCAGGAGCTGA
TGAAGAGAGAGCAGAGACAGCTCGTCTGACTTCTTTTATTGGTGCCATCGCCATTG
GAGACTTGGTAAAGAGCACCTTGGGACCCAAAGGCATGGACAAAATTCTTCTAAG
CAGTGGACGAGATGCCTCTCTTATGGTAACCAATGATGGTGCCACTATTCTAAAAA
ACATTGGTGTTGACAATCCAGCAGCTAAAGTTTTAGTTGATATGTCAAGGGTTCAA
GATGATGAAGTTGGTGATGGCACTACCTCTGTTACCGTTTTAGCAGCAGAATTATTA
AGGGAAGCAGAATCTTTAATTGCAAAAAAGATTCATCCACAGACCATCATAGCGGG
TTGGAGAGAAGCCACGAAGGCTGCAAGAGAGGCGCTGTTGAGTTCTGCAGTTGAT
CATGGTTCCGATGAAGTTAAATTCCGTCAAGATTTAATGAATATTGCGGGCACAACA
TTATCCTCAAAACTTCTTACTCATCACAAAGACCACTTTACAAAGTTAGCTGTAGA
AGCAGTTCTCAGACTGAAAGGCTCTGGCAACCTGGAGGCAATTCATATTATCAAGA
AGCTAGGAGGAAGTTTGGCAGATTCCTATTTAGATGAAGGCTTCCTGTTGGATAAA
AAAATTGGAGTAAATCAACCAAAACGAATTGAAAATGCTAAAATTCTTATTGCAAA
TACTGGTATGGATACAGACAAAATAAAGATATTTGGTTCCCGGGTAAGAGTTGACT
CTACAGCAAAGGTTGCAGAAATAGAACATGCGGAAAAGGAAAAAATGAAGGAGA
AAGTTGAACGTATTCTTAAGCATGGAATAAATTGCTTTATTAACAGGCAATTAATTTA
TAATTATCCTGAACAGCTCTTTGGTGCTGCTGGTGTCATGGCTATTGAGCATGCAGA
TTTTGCAGGTGTGGAACGCCTAGCTCTTGTCACAGGTGGTGAAATTGCCTCTACCT
TTGATCACCCAGAACTGGTGAAGCTTGGAAGTTGCAAACTTATCGAGGAAGTCAT
GATTGGAGAAGACAAACTCATTCACTTTTCTGGGGTTGCCCTTGGTGAGGCTTGTA
CCATTGTTTTGCGTGGTGCCACTCAACAAATTTTAGATGAAGCAGAAAGATCATTG
CATGATGCTCTTTGTGTTCTTGCGCAAACTGTAAAGGACTCTAGAACAGTTTATGG
AGGAGGCTGTTCTGAGATGTTGATGGCTCATGCTGTGACACAGCTTGCCAATAGAA
CACCAGGCAAAGAAGCTGTTGCAATGGAGTCTTATGCTAAAGCACTGAGAATGTT
GCCAACCATCATAGCTGACAATGCAGGCTATGACAGTGCAGACCTGGTGGCACAG
CTCAGGGCTGCTCACAGTGAAGGCAATACCACTGCTGGATTGGATATGAGGGAAG
GCACCATTGGAGATATGGCTATCCTGGGTATAACAGAAAGTTTTCAAGTGAAGCGA
CAGGTTCTTCTGAGTGCAGCTGAAGCAGCAGAGGTGATTCTGCGTGTGGACAACA
TCATCAAAGCGGCACCCAGGAAACGTGTCCCTGATCACCACCCCTGTTAA.
(SEQ ID NO: 2)
ATGGCGGCGGTGAAGACCCTGAACCCCAAGGCCGAGGTGGCCCGAGCG
CAGGCGGCGCTGGCGGTCAACATCAGCGCAGCGCGGGGTCTGCAGGACGTGCTA
AGGACCAACCTGGGGCCCAAGGGCACCATGAAGATGCTCGTTTCTGGCGCTGGAG
ACATCAAACTTACTAAAGACGGCAATGTGCTGCTTCACGAAATGCAAATTCAACAC
CCAACAGCTTCCTTAATAGCAAAGGTAGCAACAGCCCAGGATGATATAACTGGTGA
TGGTACGACTTCTAATGTCCTAATCATTGGAGAGCTGCTGAAACAGGCGGATCTCT
ACATTTCTGAAGGCCTTCATCCTAGAATAATCACTGAAGGATTTGAAGCTGCAAAG
GAAAAGGCCCTTCAGTTTTTGGAAGAAGTCAAAGTAAGCAGAGAGATGGACAGG
GAAACACTTATAGATGTGGCCAGAACATCTCTTCGTACTAAAGTTCATGCTGAACT
TGCAGATGTCTTAACAGAGGCTGTAGTGGACTCCATTTTGGCCATTAAAAAGCAAG
ATGAACCTATTGATCTCTTCATGATTGAGATCATGGAGATGAAACATAAATCTGAAA
CTGATACAAGCTTAATCAGAGGGCTTGTTTTGGACCACGGAGCACGGCATCCTGAT
ATGAAGAAAAGGGTGGAGGATGCATACATCCTCACTTGTAACGTGTCATTAGAGTA
TGAGAAAACAGAAGTGAATTCTGGCTTTTTTTACAAGAGTGCAGAAGAGAGAGA
AAAACTCGTGAAAGCTGAAAGAAAATTCATTGAAGATAGGGTTAAAAAAATAATA
GAACTGAAAAGGAAAGTCTGTGGCGATTCAGATAAAGGATTTGTTGTTATTAATCA
AAAGGGAATTGACCCCTTTTCCTTAGATGCTCTTTCAAAAGAAGGCATAGTTGCTC
TGCGCAGAGCTAAAAGGAGAAATATGGAGAGGCTGACTCTTGCTTGTGGTGGGGT
AGCCCTGAATTCTTTTGACGACCTAAGTCCTGACTGCTTGGGACATGCAGGACTTG
TATATGAGTATACATTGGGAGAAGAGAAGTTTACCTTTATTGAGAAATGTAACAACC
CTCGTTCTGTCACATTATTGATCAAAGGACCAAATAAGCACACACTCACTCAGATC
AAAGATGCAGTGAGGGACGGCTTGAGGGCTGTCAAAAATGCTATTGATGATGGCT
GTGTGGTTCCAGGTGCTGGTGCCGTGGAAGTGGCAATGGCAGAAGCCCTGATTAA
ACATAAGCCCAGTGTAAAGGGCAGGGCACAGCTTGGAGTCCAAGCATTTGCTGAT
GCATTGCTCATTATTCCCAAGGTTCTTGCTCAGAACTCTGGTTTTGACCTTCAGGAA
ACATTAGTTAAAATTCAAGCAGAACATTCAGAATCAGGTCAGCTTGTGGGTGTGGA
CCTGAACACAGGTGAGCCAATGGTGGCAGCAGAAGTAGGCGTATGGGATAACTAT
TGTGTAAAGAAACAGCTTCTTCACTCCTGCACTGTGATTGCCACCAACATTCTCTT
GGTTGATGAGATCATGCGAGCTGGAATGTCTTCTCTGAAAGGTTGA.
(SEQ ID NO: 3)
ATGGAGGGGCCTTTGTCCGTGTTCGGTGACCGCAGCACTGGGGAAACG
ATCCGCTCCCAAAACGTTATGGCTGCAGCTTCGATTGCCAATATTGTAAAAAGTTCT
CTTGGTCCAGTTGGCTTGGATAAAATGTTGGTGGATGATATTGGTGATGTAACCATT
ACTAACGATGGTGCAACCATCCTGAAGTTACTGGAGGTAGAACATCCTGCAGCTAA
AGTTCTTTGTGAGCTGGCTGATCTGCAAGACAAAGAAGTTGGAGATGGAACTACT
TCAGTGGTTATTATTGCAGCAGAACTCCTAAAAAATGCAGATGAATTAGTCAAACA
GAAAATTCATCCCACATCAGTTATTAGTGGCTATCGACTTGCTTGCAAGGAAGCAG
TGCGTTATATCAATGAAAACCTAATTGTTAACACAGATGAACTGGGAAGAGATTGC
CTGATTAATGCTGCTAAGACATCCATGTCTTCCAAAATCATTGGAATAAATGGTGAT
TTCTTTGCTAACATGGTAGTAGATGCTGTACTTGCTATTAAATACACAGACATAAGA
GGCCAGCCACGCTATCCAGTCAACTCTGTTAATATTTTGAAAGCCCATGGGAGAAG
TCAAATGGAGAGTATGCTCATCAGTGGCTATGCACTCAACTGTGTGGTGGGATCCC
AGGGCATGCCCAAGAGAATCGTAAATGCAAAAATTGCTTGCCTTGACTTCAGCCTG
CAAAAAACAAAAATGAAGCTTGGTGTACAGGTGGTCATTACAGACCCTGAAAAAC
TGGACCAAATTAGACAGAGAGAATCAGATATCACCAAGGAGAGAATTCAGAAGAT
CCTGGCAACTGGTGCCAATGTTATTCTAACCACTGGTGGAATTGATGATATGTGTCT
GAAGTATTTTGTGGAGGCTGGTGCTATGGCAGTTAGAAGAGTTTTAAAAAGGGAC
CTTAAACGCATTGCCAAAGCTTCTGGAGCAACTATTCTGTCAACCCTGGCCAATTT
GGAAGGTGAAGAAACTTTTGAAGCTGCAATGTTGGGACAGGCAGAAGAAGTGGT
ACAGGAGAGAATTTGTGATGATGAGCTGATCTTAATCAAAAATACTAAGGCTCGTA
CGTCTGCATCGATTATCTTACGTGGGGCAAATGATTTCATGTGTGATGAGATGGAGC
GCTCTTTACATGATGCACTTTGTGTAGTGAAGAGAGTTTTGGAGTCAAAATCTGTG
GTTCCCGGTGGGGGTGCTGTAGAAGCAGCCCTTTCCATATACCTTGAAAACTATGC
AACCAGCATGGGGTCTCGGGAACAGCTTGCGATTGCAGAGTTTGCAAGATCACTT
CTTGTTATTCCCAATACACTAGCAGTTAATGCTGCCCAGGACTCCACAGATCTGGTT
GCAAAATTAAGAGCTTTTCATAATGAGGCCCAGGTTAACCCAGAACGTAAAAATCT
AAAATGGATTGGTCTTGATTTGAGCAATGGTAAACCTCGAGACAACAAACAAGCA
GGGGTGTTTGAACCAACCATAGTTAAAGTTAAGAGTTTGAAATTTGCAACAGAAG
CTGCAATCACCATTCTTCGAATTGATGATCTTATTAAATTACATCCAGAAAGTAAAG
ATGATAAACATGGAAGTTATGAAGATGCTGTTCACTCTGGAGCCCTTAATGATTGA.
(SEQ ID NO: 4)
ATGATGGGCCATCGTCCAGTGCTCGTGCTCAGCCAGAACACAAAGCGTG
AATCCGGAAGAAAAGTTCAATCTGGAAACATCAATGCTGCCAAGACTATTGCAGAT
ATCATCCGAACATGTTTGGGACCCAAGTCCATGATGAAGATGCTTTTGGACCCAAT
GGGAGGCATTGTGATGACCAATGATGGCAATGCCATTCTTCGAGAGATTCAAGTCC
AGCATCCAGCGGCCAAGTCCATGATCGAAATTAGCCGGACCCAGGATGAAGAGGT
TGGAGATGGGACCACATCAGTAATTATTCTTGCAGGGGAAATGCTGTCTGTAGCTG
AGCACTTCCTGGAGCAGCAGATGCACCCAACAGTGGTGATCAGTGCTTACCGCAA
GGCATTGGATGATATGATCAGCACCCTAAAGAAAATAAGTATCCCAGTCGACATCA
GTGACAGTGATATGATGCTGAACATCATCAACAGCTCTATTACTACCAAAGCCATCA
GTCGGTGGTCATCTTTGGCTTGCAACATTGCCCTGGATGCTGTCAAGATGGTACAG
TTTGAGGAGAATGGTCGGAAAGAGATTGACATAAAAAAATATGCAAGAGTGGAAA
AGATACCTGGAGGCATCATTGAAGACTCCTGTGTCTTGCGTGGAGTCATGATTAAC
AAGGATGTGACCCATCCACGTATGCGGCGCTATATCAAGAACCCTCGCATTGTGCT
GCTGGATTCTTCTCTGGAATACAAGAAAGGAGAAAGCCAGACTGACATTGAGATT
ACACGAGAGGAGGACTTCACCCGAATTCTCCAGATGGAGGAAGAGTACATCCAGC
AGCTCTGTGAGGACATTATCCAACTGAAGCCCGATGTGGTCATCACTGAAAAGGG
CATCTCAGATTTAGCTCAGCACTACCTTATGCGGGCCAATATCACAGCCATCCGCAG
AGTCCGGAAGACAGACAATAATCGCATTGCTAGAGCCTGTGGGGCCCGGATAGTC
AGCCGACCAGAGGAACTGAGAGAAGATGATGTTGGAACAGGAGCAGGCCTGTTG
GAAATCAAGAAAATTGGAGATGAATACTTTACTTTCATCACTGACTGCAAAGACCC
CAAGGCCTGCACCATTCTCCTCCGGGGGGCTAGCAAAGAGATTCTCTCGGAAGTA
GAACGCAACCTCCAGGATGCCATGCAAGTGTGTCGCAATGTTCTCCTGGACCCTCA
GCTGGTGCCAGGGGGTGGGGCCTCCGAGATGGCTGTGGCCCATGCCTTGACAGAA
AAATCCAAGGCCATGACTGGTGTGGAACAATGGCCATACAGGGCTGTTGCCCAGG
CCCTAGAGGTCATTCCTCGTACCCTGATCCAGAACTGTGGGGCCAGCACCATCCGT
CTACTTACCTCCCTTCGGGCCAAGCACACCCAGGAGAACTGTGAGACCTGGGGTG
TAAATGGTGAGACGGGTACTTTGGTGGACATGAAGGAACTGGGCATATGGGAGCC
ATTGGCTGTGAAGCTGCAGACTTATAAGACAGCAGTGGAGACGGCAGTTCTGCTA
CTGCGAATTGATGACATCGTTTCAGGCCACAAAAAGAAAGGCGATGACCAGAGCC
GGCAAGGCGGGGCTCCTGATGCTGGCCAGGAGTGA.
(SEQ ID NO: 5)
ATGATAAGTGCCAGCCGAGCTGCAGCAGCCCGTCTCGTGGGCGCCGCA
GCCTCCCGGGGCCCTACGGCCGCCCGCCACCAGGATAGCTGGAATGGCCTTAGTCA
TGAGGCTTTTAGACTTGTTTCAAGGCGGGATTATGCATCAGAAGCAATCAAGGGAG
CAGTTGTTGGTATTGATTTGGGTACTACCAACTCCTGCGTGGCAGTTATGGAAGGT
AAACAAGCAAAGGTGCTGGAGAATGCCGAAGGTGCCAGAACCACCCCTTCAGTT
GTGGCCTTTACAGCAGATGGTGAGCGACTTGTTGGAATGCCGGCCAAGCGACAGG
CTGTCACCAACCCAAACAATACATTTTATGCTACCAAGCGTCTCATTGGCCGGCGAT
ATGATGATCCTGAAGTACAGAAAGACATTAAAAATGTTCCCTTTAAAATTGTCCGT
GCCTCCAATGGTGATGCCTGGGTTGAGGCTCATGGGAAATTGTATTCTCCGAGTCA
GATTGGAGCATTTGTGTTGATGAAGATGAAAGAGACTGCAGAAAATTACTTGGGG
CACACAGCAAAAAATGCTGTGATCACAGTCCCAGCTTATTTCAATGACTCGCAGAG
ACAGGCCACTAAAGATGCTGGCCAGATATCTGGACTGAATGTGCTTCGGGTGATTA
ATGAGCCCACAGCTGCTGCTCTTGCCTATGGTCTAGACAAATCAGAAGACAAAGTC
ATTGCTGTATATGATTTAGGTGGTGGAACTTTTGATATTTCTATCCTGGAAATTCAGA
AAGGAGTATTTGAGGTGAAATCCACAAATGGGGATACCTTCTTAGGTGGGGAAGA
CTTTGACCAGGCCTTGCTACGGCACATTGTGAAGGAGTTCAAGAGAGAGACAGGG
GTTGATTTGACTAAAGACAACATGGCACTTCAGAGGGTACGGGAAGCTGCTGAAA
AGGCTAAATGTGAACTCTCCTCATCTGTGCAGACTGACATCAATTTGCCCTATCTTA
CAATGGATTCTTCTGGACCCAAGCATTTGAATATGAAGTTGACCCGTGCTCAATTTG
AAGGGATTGTCACTGATCTAATCAGAAGGACTATCGCTCCATGCCAAAAAGCTATG
CAAGATGCAGAAGTCAGCAAGAGTGACATAGGAGAAGTGATTCTTGTGGGTGGCA
TGACTAGGATGCCCAAGGTTCAGCAGACTGTACAGGATCTTTTTGGCAGAGCCCC
AAGTAAAGCTGTCAATCCTGATGAGGCTGTGGCCATTGGAGCTGCCATTCAGGGA
GGTGTGTTGGCCGGCGATGTCACGGATGTGCTGCTCCTTGATGTCACTCCCCTGTC
TCTGGGTATTGAAACTCTAGGAGGTGTCTTTACCAAACTTATTAATAGGAATACCAC
TATTCCAACCAAGAAGAGCCAGGTATTCTCTACTGCCGCTGATGGTCAAACGCAAG
TGGAAATTAAAGTGTGTCAGGGTGAAAGAGAGATGGCTGGAGACAACAAACTCC
TTGGACAGTTTACTTTGATTGGAATTCCACCAGCCCCTCGTGGAGTTCCTCAGATT
GAAGTTACATTTGACATTGATGCCAATGGGATAGTACATGTTTCTGCTAAAGATAAA
GGCACAGGACGTGAGCAGCAGATTGTAATCCAGTCTTCTGGTGGATTAAGCAAAG
ATGATATTGAAAATATGGTTAAAAATGCAGAGAAATATGCTGAAGAAGACCGGCGA
AAGAAGGAACGAGTTGAAGCAGTTAATATGGCTGAAGGAATCATTCACGACACAG
AAACCAAGATGGAAGAATTCAAGGACCAATTACCTGCTGATGAGTGCAACAAGCT
GAAAGAAGAGATTTCCAAAATGAGGGAGCTCCTGGCTAGAAAAGACAGCGAAAC
AGGAGAAAATATTAGACAGGCAGCATCCTCTCTTCAGCAGGCATCATTGAAGCTGT
TCGAAATGGCATACAAAAAGATGGCATCTGAGCGAGAAGGCTCTGGAAGTTCTGG
CACTGGGGAACAAAAGGAAGATCAAAAGGAGGAAAAACAGTAA.
(SEQ ID NO: 6)
ATGCCTGAGGAAGTGCACCATGGAGAGGAGGAGGTGGAGACTTTTGCC
TTTCAGGCAGAAATTGCCCAACTCATGTCCCTCATCATCAATACCTTCTATTCCAAC
AAGGAGATTTTCCTTCGGGAGTTGATCTCTAATGCTTCTGATGCCTTGGACAAGATT
CGCTATGAGAGCCTGACAGACCCTTCGAAGTTGGACAGTGGTAAAGAGCTGAAAA
TTGACATCATCCCCAACCCTCAGGAACGTACCCTGACTTTGGTAGACACAGGCATT
GGCATGACCAAAGCTGATCTCATAAATAATTTGGGAACCATTGCCAAGTCTGGTAC
TAAAGCATTCATGGAGGCTCTTCAGGCTGGTGCAGACATCTCCATGATTGGGCAGT
TTGGTGTTGGCTTTTATTCTGCCTACTTGGTGGCAGAGAAAGTGGTTGTGATCACA
AAGCACAACGATGATGAACAGTATGCTTGGGAGTCTTCTGCTGGAGGTTCCTTCAC
TGTGCGTGCTGACCATGGTGAGCCCATTGGCAGGGGTACCAAAGTGATCCTCCATC
TTAAAGAAGATCAGACAGAGTACCTAGAAGAGAGGCGGGTCAAAGAAGTAGTGA
AGAAGCATTCTCAGTTCATAGGCTATCCCATCACCCTTTATTTGGAGAAGGAACGA
GAGAAGGAAATTAGTGATGATGAGGCAGAGGAAGAGAAAGGTGAGAAAGAAGA
GGAAGATAAAGATGATGAAGAAAAACCCAAGATCGAAGATGTGGGTTCAGATGAG
GAGGATGACAGCGGTAAGGATAAGAAGAAGAAAACTAAGAAGATCAAAGAGAAA
TACATTGATCAGGAAGAACTAAACAAGACCAAGCCTATTTGGACCAGAAACCCTG
ATGACATCACCCAAGAGGAGTATGGAGAATTCTACAAGAGCCTCACTAATGACTGG
GAAGACCACTTGGCAGTCAAGCACTTTTCTGTAGAAGGTCAGTTGGAATTCAGGG
CATTGCTATTTATTCCTCGTCGGGCTCCCTTTGACCTTTTTGAGAACAAGAAGAAA
AAGAACAACATCAAACTCTATGTCCGCCGTGTGTTCATCATGGACAGCTGTGATGA
GTTGATACCAGAGTATCTCAATTTTATCCGTGGTGTGGTTGACTCTGAGGATCTGCC
CCTGAACATCTCCCGAGAAATGCTCCAGCAGAGCAAAATCTTGAAAGTCATTCGC
AAAAACATTGTTAAGAAGTGCCTTGAGCTCTTCTCTGAGCTGGCAGAAGACAAGG
AGAATTACAAGAAATTCTATGAGGCATTCTCTAAAAATCTCAAGCTTGGAATCCAC
GAAGACTCCACTAACCGCCGCCGCCTGTCTGAGCTGCTGCGCTATCATACCTCCCA
GTCTGGAGATGAGATGACATCTCTGTCAGAGTATGTTTCTCGCATGAAGGAGACAC
AGAAGTCCATCTATTACATCACTGGTGAGAGCAAAGAGCAGGTGGCCAACTCAGC
TTTTGTGGAGCGAGTGCGGAAACGGGGCTTCGAGGTGGTATATATGACCGAGCCCA
TTGACGAGTACTGTGTGCAGCAGCTCAAGGAATTTGATGGGAAGAGCCTGGTCTC
AGTTACCAAGGAGGGTCTGGAGCTGCCTGAGGATGAGGAGGAGAAGAAGAAGAT
GGAAGAGAGCAAGGCAAAGTTTGAGAACCTCTGCAAGCTCATGAAAGAAATCTT
AGATAAGAAGGTTGAGAAGGTGACAATCTCCAATAGACTTGTGTCTTCACCTTGCT
GCATTGTGACCAGCACCTACGGCTGGACAGCCAATATGGAGCGGATCATGAAAGC
CCAGGCACTTCGGGACAACTCCACCATGGGCTATATGATGGCCAAAAAGCACCTG
GAGATCAACCCTGACCACCCCATTGTGGAGACGCTGCGGCAGAAGGCTGAGGCCG
ACAAGAATGATAAGGCAGTTAAGGACCTGGTGGTGCTGCTGTTTGAAACCGCCCT
GCTATCTTCTGGCTTTTCCCTTGAGGATCCCCAGACCCACTCCAACCGCATCTATCG
CATGATCAAGCTAGGTCTAGGTATTGATGAAGATGAAGTGGCAGCAGAGGAACCC
AATGCTGCAGTTCCTGATGAGATCCCCCCTCTCGAGGGCGATGAGGATGCGTCTCG
CATGGAAGAAGTCGATTAG.

According to an embodiment of the present invention, the expression vector is AAV.

According to an embodiment of the present invention, the method is independent of cargo ubiquitination. The inventor found that depletion of key factors of the cargoes ubiquitination did not affect the association of CCT2 with ATG8, therefore, the method according to the embodiment of the present inventions independent of cargo ubiquitination.

According to an embodiment of the present invention, the method is realized through autophagy.

According to an embodiment of the present invention, the activity of chaperones is the ability of chaperones to degrade solid protein aggregates and/or aggregation-prone proteins by autophagy.

In one aspect of present disclosure, use of reagents in the preparation of drugs for the treatment or prevention of diseases caused by protein aggregation is provided. Wherein the reagents are used for at least one of the following: overexpressing chaperones or enhancing the activity of chaperones; enhancing the chaperones interaction with solid protein aggregates and/or aggregation-prone proteins; enhancing the chaperones interaction with ATG8s; promoting the disassociation of TRIC to produce free subunits; overexpressing/applying the D2 and/or D3 domain of CCT2 or enhancing the D2 and/or D3 domain activity of CCT2; overexpressing/applying the P7 Peptide of CCT2 or enhancing the P7 Peptide activity of CCT2; enhancing the activity of amino acids 503 ˜505 and/or 513 ˜515 of CCT2; overexpressing/applying the peptide or enhancing the peptide activity, wherein the peptide comprises amino acids 503 to 515 of CCT2 and optionally at least 10 amino acids upstream of amino acid 503 or at least 10 amino acids downstream of amino acid 515. As described above, the reagents can promote the removal of solid protein aggregates and/or aggregation-prone proteins, therefore, the drugs with the regents can treat or prevent diseases caused by protein aggregation effectively.

According to an embodiment of the present invention, the diseases caused by protein aggregation including at least one of the following: neurodegenerative diseases, eye disease, and type II diabetes, amyloid transthyretin cardiomyopathy.

According to an embodiment of the present invention, the neurodegenerative diseases include at least one of the following: Alzheimer's disease (AD), Parkinson's disease (PD), Huntington's disease (HD), amyotrophic lateral sclerosis (ALS), different types of spinocerebellar ataxia (SCA), pick disease, dementia with Lewy bodies, frontotemporal dementia.

According to an embodiment of the present invention, the chaperone comprises at least one of the following: CCT2, CCT6, CCT1, CCT3, HSPA9, and HSP90AB1.

According to an embodiment of the present invention, the free subunits comprise at least one of the following: CCT2, CCT6, CCT1, CCT3.

According to an embodiment of the present invention, the reagent comprises expression vector with chaperones coding nucleic acid or compounds, protein or factors used for enhancing the activity of chaperones.

According to an embodiment of the present invention, the reagent comprises expression vector with D2 and/or D3 domain coding nucleic acid or compounds, protein or factors used for enhancing the activity of D2 and/or D3 domain.

According to an embodiment of the present invention, the CCT2 coding nucleic acid has the nucleotide sequence shown in SEQ ID No: 1 or CCT6 coding nucleic acid has the nucleotide sequence shown in SEQ ID No: 2; or CCT1 coding nucleic acid has the nucleotide sequence shown in SEQ ID No: 3; or CCT3 coding nucleic acid has the nucleotide sequence shown in SEQ ID No: 4; or HSPA9 coding nucleic acid has the nucleotide sequence shown in SEQ ID No: 5; or HSP90AB1 coding nucleic acid has the nucleotide sequence shown in SEQ ID No: 6.

According to an embodiment of the present invention, the expression vector is AAV.

In one aspect of present disclosure, a method for treating or preventing of diseases caused by protein aggregation comprising: Administration medication to subjects, wherein the medication is used for at least one of the following: overexpressing chaperones or enhancing the activity of chaperones; enhancing the chaperones interaction with solid protein aggregates and/or aggregation-prone proteins; enhancing the chaperones interaction with ATG8s; promoting the degradation of TRIC to produce free subunits; overexpressing the D2 and/or D3 domain of CCT2 or enhancing the D2 and/or D3 domain activity of CCT2; overexpressing/applying the P7 Peptide of CCT2 or enhancing the P7 Peptide activity of CCT2; enhancing the activity of amino acids 503˜505 and/or 513˜515 of CCT2; overexpressing/applying the peptide or enhancing the peptide activity, wherein the peptide comprises amino acids 503 to 515 of CCT2 and optionally at least 10 amino acids upstream of amino acid 503 or at least 10 amino acids downstream of amino acid 515. As described above, the medication described above can promote the removal of solid protein aggregates and/or aggregation-prone proteins, therefore, the method can treat or prevent diseases caused by protein aggregation effectively.

According to an embodiment of the present invention, the administration is by injection.

According to an embodiment of the present invention, the injection is in situ or intravenous administration.

According to an embodiment of the present invention, the diseases caused by protein aggregation including at least one of the following: neurodegenerative diseases, eye disease, type II diabetes and amyloid transthyretin cardiomyopathy.

According to an embodiment of the present invention, the neurodegenerative diseases include at least one of the following: Alzheimer's disease (AD), Parkinson's disease (PD), Huntington's disease (HD), amyotrophic lateral sclerosis (ALS), dementia with Lewy bodies, frontotemporal dementia, different types of spinocerebellar ataxia (SCA), pick disease.

In one aspect of present disclosure, a method for screening drugs for treatment or prevention diseases caused by protein aggregation is provided, wherein the method comprises: contact the model with the drug to be screened, and compare the changes of at least one of the following before and after contact in the model: the expression quantity of chaperones or the activity of chaperones; the binding force of chaperones with ATG8s; the binding force of chaperones with solid protein aggregates and/or aggregation-prone proteins; the quantity of TRIC free subunits; the expression quantity of the D2 and/or D3 domain of CCT2 or the activity of the D2 and/or D3 domain of CCT2; the expression quantity of the P7 Peptide of CCT2 or the activity of P7 Peptide of CCT2; the activity of amino acids 503 ˜505 and/or 513 ˜515 of CCT2; the expression quantity of the peptide or the activity of the peptide, wherein the peptide comprises amino acids 503 to 515 of CCT2 and optionally at least 10 amino acids upstream of amino acid 503 or at least 10 amino acids downstream of amino acid 515; and based on the change, determine whether the drug to be screened is the target drug. As described above, chaperones, such as CCT2, are as a new autophagy receptor and responsible for clearance of solid protein aggregates and/or aggregation-prone proteins. Therefore, during screening drugs for treatment or prevention diseases caused by protein aggregation, the chaperones related change could be the hallmarker of the target drug. According to an embodiment of the present invention, the method described above can screen drugs for treatment or prevention diseases caused by protein aggregation effectively.

According to an embodiment of the present invention, after exposure compared with before exposure, a rise in at least one of the following: the expression quantity of chaperones or the activity of chaperones; the binding force of chaperones with ATG8s; the binding force of chaperones with solid protein aggregates and/or aggregation-prone proteins; the quantity of TRIC free subunits; the expression quantity of the D2 and/or D3 domain of CCT2 or the activity of the D2 and/or D3 domain of CCT2; the expression quantity of the P7 Peptide of CCT2 or the activity of P7 Peptide of CCT2; the activity of amino acids 503 ˜505 and/or 513 ˜515 of CCT2; the expression quantity of the peptide or the activity of the peptide, wherein the peptide comprises amino acids 503 to 515 of CCT2 and optionally at least 10 amino acids upstream of amino acid 503 or at least 10 amino acids downstream of amino acid 515; is an indication that the drug to be screened is the target drug.

According to an embodiment of the present invention, the chaperone comprises at least one of the following: CCT2, CCT6, CCT1, CCT3, HSPA9 and HSP90AB1.

According to an embodiment of the present invention, after exposure compared with before exposure, a rise in at least one of the following: the expression quantity of CCT2 or the activity of CCT2; the binding force of CCT2 with ATG8s; the binding force of CCT2 with solid protein aggregates and/or aggregation-prone proteins; the expression quantity of the P7 Peptide of CCT2 or the activity of P7 Peptide of CCT2; the activity of amino acids 503 ˜505 and/or 513 ˜515 of CCT2; the expression quantity of the peptide or the activity of the peptide, wherein the peptide comprises amino acids 503 to 515 of CCT2 and optionally at least 10 amino acids upstream of amino acid 503 or at least 10 amino acids downstream of amino acid 515; is an indication that the drug to be screened is the target drug.

According to an embodiment of the present invention, after exposure compared with before exposure, a rise in at least one of the following: the expression quantity of CCT6 or the activity of CCT6; the binding force of CCT6 with ATG8s; the binding force of CCT6 with solid protein aggregates and/or aggregation-prone proteins; is an indication that the drug to be screened is the target drug.

According to an embodiment of the present invention, after exposure compared with before exposure, a rise in at least one of the following: the expression quantity of CCT1 or the activity of CCT1; the binding force of CCT1 with ATG8s; the binding force of CCT1 with solid protein aggregates and/or aggregation-prone proteins; is an indication that the drug to be screened is the target drug.

According to an embodiment of the present invention, after exposure compared with before exposure, a rise in at least one of the following: the expression quantity of CCT3 or the activity of CCT3; the binding force of CCT3 with ATG8s; the binding force of CCT3 with solid protein aggregates and/or aggregation-prone proteins; is an indication that the drug to be screened is the target drug.

According to an embodiment of the present invention, after exposure compared with before exposure, a rise in at least one of the following: the expression quantity of HSPA9 or the activity of HSPA9; the binding force of HSPA9 with ATG8s; the binding force of HSPA9 with solid protein aggregates and aggregation-prone proteins; is an indication that the drug to be screened is the target drug.

According to an embodiment of the present invention, after exposure compared with before exposure, a rise in at least one of the following: the expression quantity of HSP90AB1 or the activity of HSP90AB1; the binding force of HSP90AB1 with solid protein aggregates and aggregation-prone proteins; is an indication that the drug to be screened is the target drug.

According to an embodiment of the present invention, the model is cultured cell lines, nerve cell, tissue or mice.

According to an embodiment of the present invention, the model is CCT2 knockdown or overexpression cultured cell lines, nerve cell, tissue or mice.

According to an embodiment of the present invention, the cultured cell lines, nerve cell or tissue has solid protein aggregates and/or aggregation-prone proteins.

According to an embodiment of the present invention, the diseases caused by protein aggregation including at least one of the following: neurodegenerative diseases, eye disease, type II diabetes, and amyloid transthyretin cardiomyopathy.

According to an embodiment of the present invention, the neurodegenerative diseases include at least one of the following: Alzheimer's disease (AD), Parkinson's disease (PD), Huntington's disease (HD), amyotrophic lateral sclerosis (ALS), dementia with Lewy bodies, frontotemporal dementia, different types of spinocerebellar ataxia (SCA), pick disease.

In one aspect of present disclosure, a fusion protein is provided, wherein the fusion protein comprises: a first peptide segment and a second peptide segment, wherein the first peptide segment comprising D2 domain of CCT2 and the second peptide segment comprising D3 domain of CCT2 or P7 peptide of CCT2. The inventors identify a new function of the chaperones in aggrephagy. The chaperonin subunit, such as CCT2, is as a new autophagy receptor regulating the clearance of aggregation-prone proteins in cell and mouse brain. The fusion protein containing the D2 domain of CCT2, and the D3 domain of CCT2 or P7 peptide of CCT2 specifically promotes the autophagic degradation of protein aggregates with little liquidity (solid aggregates).

According to an embodiment of the present invention, the C-terminal of the first peptide segment is connected with the N-terminal of the second peptide segment.

According to an embodiment of the present invention, the fusion protein further comprising a connecting peptide arranged between the first peptide segment and the second peptide segment.

According to an embodiment of the present invention, the N-terminal of the connecting peptide is connected with the C-terminal of the first peptide segment, and the C-terminal of the connecting peptide is connected with the N-terminal of the second peptide segment.

According to an embodiment of the present invention, the fusion protein has the amino acid sequence of SEQ ID NO: 13 or 14.

In another aspect of present disclosure, a nucleic acid is provided, wherein the nucleic acid encoding the fusion protein. As mentioned above, the fusion protein encoded by the nucleic acids specifically promotes the autophagic degradation of protein aggregates with little liquidity (solid aggregates).

According to an embodiment of the present invention, the nucleic acid has the nucleotide sequence of SEQ ID NO: 15 or 16.

It would be appreciated that the nucleic acid, as mentioned in the description and claims of the present disclosure, includes any one, or two, of a complementary double-strand. In the description and claims of the present disclosure, only one strand is provided in most cases for convenience, but the disclosure includes the other one strand of the complementary double-strand. For example, when referring to SEQ ID NO: 15 to 16, they include their complementary sequences. It would be also understood that one strand can be determined using the other one strand of the complementary double-strand, vice versa.

The gene sequence in the present disclosure includes both the DNA form and the RNA form, wherein in the case that one form is disclosed, the other one is also disclosed.

The term “encoding” refers to the inherent properties of polynucleotides such as genes, cDNAs, or mRNAs in which specific nucleotide sequences are used as templates for the synthesis of other polymers and macromolecules in biological processes. The polymers and macromolecules have a certain nucleotide sequence (e.g. rRNA, tRNA, and mRNA) or defined amino acid sequence and the resulting biological properties. Therefore, if the transcription and translation of mRNA corresponding to a gene produces a protein in a cell or other biological system, the gene, cDNA, or RNA encodes the protein. The coding strand and its nucleotide sequence are identical to the mRNA sequence and are usually provided in the sequence listing, while the non-coding strand used as a template for the transcription of a gene or cDNA can be referred to as a coding protein or other products of the gene or cDNA. Unless otherwise specified, “nucleotide sequence encoding an amino acid sequence” includes all nucleotide sequences that are degenerate forms of each other and encode the same amino acid sequence.

In one aspect of present disclosure, a construct is provided, wherein the construct carrying the nucleic acid. As mentioned above, the fusion protein encoded by the nucleic acids specifically promotes the autophagic degradation of protein aggregates with little liquidity (solid aggregates). The construct used in the present invention can effectively realize the expression of the fusion protein mentioned above under the mediation of the regulatory system after introducing appropriate receptor cells, and then achieve the large amount of the fusion protein in vitro.

The term “construct” used in present disclosure refers to a genetic vector containing a recombinant polynucleotide comprising an expression control sequence operably linked to the nucleotide sequence to be expressed, and capable of transferring a targeting nucleic acid sequence into a host cell to obtain a recombinant cell. The construct according to the embodiments of present disclosure is not specifically limited in any form. In some embodiments, the construct can be at least one of plasmid, bacteriophage, artificial chromosome, cosmid and virus, preferably plasmid. As a genetic vector, the plasmid is easy to deal with and can carry larger fragment, which is beneficial to further manipulate and treat. The plasmid is also not specifically limited in any form and can be a circular plasmid or linear plasmid, single-strand or double-strand, which can be selected by a person skilled in the art depending on actual requirement. The term “nucleic acid” used herein can be any polymer containing deoxyribonucleotides or ribonucleotides, including but not necessarily limited to modified or unmodified DNA and RNA, and shall has no specific limits to its length. The nucleic acid, for the construct for constructing the recombinant cell, is preferably DNA as it's more stable and easier for operation compared to RNA.

In one aspect of present disclosure, a recombinant cell is provided, wherein the recombinant cell carrying the nucleic acid or the construct or expressing the fusion protein. The recombinant cell effectively realizes the expression of the fusion protein mentioned above under appropriate conditions, and then achieve the in vitro availability of the fusion protein in large quantities.

The term “expression” refers to the transcription and/or translation of a specific nucleotide sequence driven by a promoter.

In another aspect of present disclosure, use of fusion protein in the preparation of drugs used for treatment or prevention diseases caused by protein aggregation. As mentioned above, CCT2, is as a new autophagy receptor regulating the clearance of aggregation-prone proteins in cell and mouse brain. Furthermore, drugs containing the fusion protein can be effectively treated or prevented diseases caused by protein aggregation.

The drugs of the present disclosure contain fusion protein thereof as described herein, and appropriate carriers including, for example, pharmaceutically acceptable carriers or diluents.

In some embodiments, carriers include pharmaceutically acceptable carriers, excipients, or stabilizers that are nontoxic to the cell or subject being exposed thereto at the dosages and concentrations employed. Often the physiologically acceptable carrier is an aqueous pH buffered solution. Suitable physiologically acceptable carriers include, for example, buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™

Suitable formulations include, for example, solutions, injections. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Such diluents include, for example, distilled water, buffered water, physiological saline, PBS, Ringer's solution, dextrose solution, and Hank's solution. A pharmaceutical composition or formulation of the present disclosure can further include, for example, other carriers or non-toxic, nontherapeutic, nonimmunogenic stabilizers, and excipients. The drugs can also include additional substances to approximate physiological conditions, such as pH adjusting and buffering agents, toxicity adjusting agents, wetting agents and detergents. A drug of the present disclosure can also include any of a variety of stabilizing agents, such as an antioxidant for example.

Drugs of the present disclosure can be suitable for oral or intestinal administration. In some embodiments, the drugs of are used (e.g., administered to a subject in need of treatment, such as a human individual) by oral administration. For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Dosages and desired concentration of drugs of the present disclosure may vary depending on the particular use envisioned. The determination of the appropriate dosage or route of administration is well within the skill of an ordinary artisan.

Administration of a drug of the present disclosure can be continuous or intermittent, depending, for example, on the recipient's physiological condition, whether the purpose of the administration is therapeutic or prophylactic, and other factors known to skilled practitioners. It is within the scope of the present disclosure that dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

According to an embodiment of the present invention, the diseases caused by protein aggregation including at least one of the following: neurodegenerative diseases, eye disease, type II diabetes and amyloid transthyretin cardiomyopathy.

According to an embodiment of the present invention, the neurodegenerative diseases include at least one of the following: Alzheimer's disease (AD), Parkinson's disease (PD), Huntington's disease (HD), amyotrophic lateral sclerosis (ALS), dementia with Lewy bodies, frontotemporal dementia, different types of spinocerebellar ataxia (SCA), pick disease.

More aspects and advantages will be described below, at least a part there of will be clear in the following description accompanying the figures as attached, and/or be obvious for a person normally skilled in the art from embodiments described herein after.

### BRIEF DESCRIPTION OF THE FIGURES

The aforementioned features and advantages of the invention as well as additional features and advantages thereof will be more clearly understood hereafter as a result of a detailed description of the following embodiments when taken conjunction with the drawings, wherein:

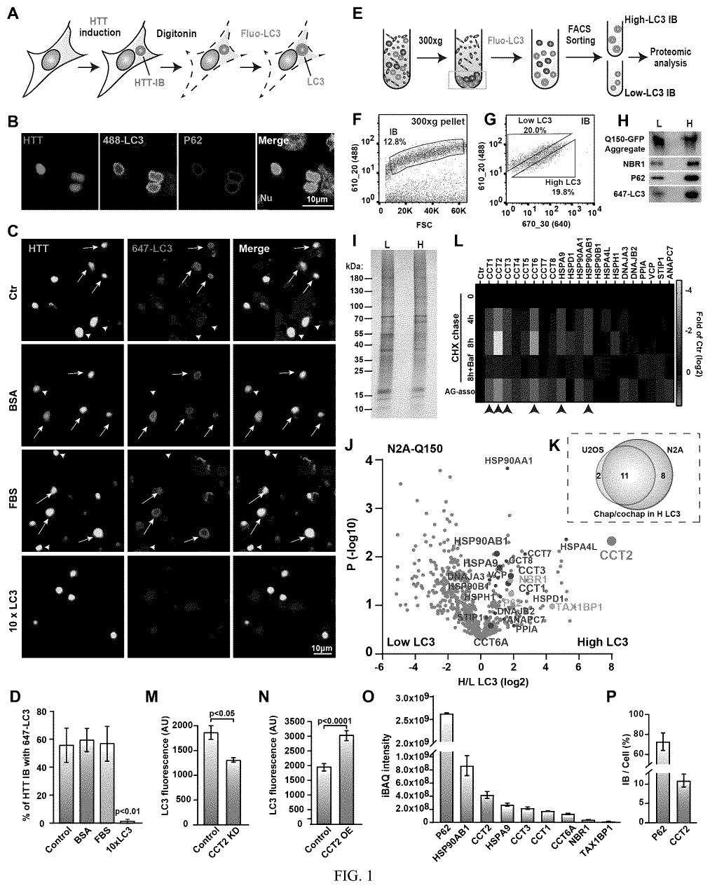

FIG. 1 shows identification of CCT2 and the other chaperones as proteins involved in LC3 recruitment to IBs, wherein, (A) Schematic diagram of in vitro reconstitution system for analyzing LC3 recruitment to the IB in the cells.
(B) Confocal images showing the recruitment of fluorescent LC3 to IBs in U2OS Q91-HTT-mCherry cells.
(C) Confocal images showing the competition of non-fluorescent LC3 against fluorescent LC3 recruitment to IBs in N2A Q150-HTT-GFP cells. Arrows and arrowheads point to IBs with high (H) and low (L) LC3 association respectively.
(D) Quantification of the percentage of IB with fluorescent LC3 (mean±SD) as shown in (C). P values are indicated (two-tailed t test, >100 IBs from three independent experiments).
(E) Schematic diagram of FAPS. The cells were lysed and IBs were enriched by centrifuging at 300 xg. LC3 recruitment was performed using the IB-enriched pellet and FACS sorting was employed to obtain IBs with H- and L-LC3 recruitment.
(F) FACS chart showing the gating of IB-fraction in FAPS.
(G) FACS chart showing gating of IBs with H- and L-LC3 in FAPS.
(H) Immunoblot analyzing the indicated proteins in IBs with H- and L-LC3.
(I) Silver staining of IBs with H- and L-LC3.
(J) Volcano Plot showing the different protein enrichment in IBs with H- and L-LC3.
(K) Venn diagram showing the amount of chaperones/cochaperones enriched in H-LC3 IBs as well as the overlap in U2OS and N2A cells.
(L) Heatmap showing the lysosome-dependent clearance of Q103-HTT and LC3 puncta association with IBs upon expression of the indicated chaperones/cochaperones.
(M) Quantification of LC3 fluorescence (mean±SD) on IBs by FACS in control or CCT2 KD condition. P values are indicated (two-tailed t test, three independent experiments).
(N) Quantification of LC3 fluorescence (mean±SD) on IBs by FACS in control or CCT2 expression condition. P values are indicated (two-tailed t test, four independent experiments).
(O) Quantification of indicated chaperones and autophagy receptors (mean±SD) on IBs by unlabeled quantitative mass spectrometry.
(P) The percentage of cellular P62 and CCT2 (mean±SD) on IBs quantified by unlabeled quantitative mass spectrometry.

FIG. 2 shows CCT2 regulates autophagic degradation of polyQ-HTT, wherein, (A) Immunofluorescence of U2OS co-expressing Q103-HTT-BFP and mCherry or HA-CCT2 with anti-HA and LC3 antibodies. The cells were permeabilized with digitonin before fixation.
(B) Quantification of LC3 around Q103 IB (mean±SEM) as shown in (A). P values are indicated (two-tailed t test, >50 cells from three independent experiments).
(C) Immunofluorescence of U2OS co-expressing Q103-HTT-BFP, HA-CCT2, and mCherry-LC3B WT or G120A with anti-HA antibodies. The cells were permeabilized with or without digitonin as indicated before fixation.
(D) Quantification of digitonin-insoluble LC3 around Q103 IB (mean±SEM) as shown in (C). P values are indicated (two-tailed t test, >50 cells from three independent experiments).
(E) Immunofluorescence of MEF WT or Atg5KO cells co-expressing Q103-HTT-BFP and HA-CCT2 with anti-HA and LC3 antibodies.
(F) Quantification of LC3 around Q103 IB (mean±SEM) as shown in (E). P values are indicated (two-tailed t test, >50 cells from three independent experiments).
(G) Immunofluorescence of U2OS co-expressing Q103-HTT-BFP and GFP-CCT2 with anti-LC3 and LAMP2 antibodies. The cells were permeabilized with digitonin before fixation.
(H) Electron microscopy of the APEX2-labeled Q103-HTT IBs and the autophagosomes with or without HA-CCT2 expression in U2OS. Cells were pre-transfected with siRNA against Atg5 before, or post-treated with 5 μM SAR405 for 12h after Q103-HTT-APEX2 and HA-CCT2 expression as indicated.
(I) Quantification of numbers of the Q103-HTT-positive autophagosomes in cells with IBs (mean±SEM) as shown in (H). P values are indicated (one-way ANOVA, >30 cells with IBs from three independent experiments).
(J) Membrane fractionation scheme to isolate autophagosomes.
(K) HEK293T cells were transfected with Q103-HTT-GFP and HA-CCT2. Immunoblot was performed to examine the distribution of Q103 and HA-CCT2 in the OptiPrep gradient fractions 1-10 as shown in (J). F-AG: autophagosome fractions. The data are representative of three independent experiments.
(L) Q103-HTT-GFP was expressed with or without HA-CCT2 in HEK293T. The F-AG as shown in (K) was isolated and treated with or without proteinase K and Triton X-100 as indicated. The indicated proteins were determined by immunoblot. The numbers indicate normalized Q103 to LC3-II ratio, in which the ratio of Q103-GFP to LC3-II in the autophagosome fraction from the control group was set as 1. The data are representative of three independent experiments.
(M) Immunoblot of striatum from AAV-mCherry- or AAV-CCT2-injected Hdh140Q mice at 2 months post AAV injection. The mice were injected with the AAVs at the age of 2 months. The data show 2 mice representative of 6 mice in the experiment.

(N) Analysis of Q103-HTT degradation in a CHX chase assay with or without HA-CCT2 expression in U2OS pre-transfected with siRNA against control, Atg5 or Beclin-1.
(O) Quantification of normalized Q103-HTT (mean±SEM) in (N). P values are indicated (two-way ANOVA, two independent experiments).

FIG. 3 shows CCT2 is required for polyQ-HTT degradation, wherein, (A) Immunofluorescence of U2OS expressing Q103-HTT-BFP with anti-CCT2 and LC3 antibodies.
(B) Quantification of CCT2 and LC3 around Q103 IB (mean±SEM) as shown in (A). P values are indicated (two-tailed t test, >50 cells from three independent experiments).
(C) Electron microscopy of Q103-HTT IBs with or without HA-CCT2 expression in U2OS.
(D) Quantification of the number of autophagic vacuole-like vesicles with IB (mean±SEM) as shown in (C). P values are indicated (two-tailed t test, >20 IBs from three independent experiments).
(E) CLEM imaging of U2OS expressing Q103-HTT-BFP, GFP-CCT2, and mCherry-LC3.
(F) Immunofluorescence of U2OS cell co-expressing Q103-HTT-BFP and GFP-CCT2 with anti-LC3 and FIP200 antibodies. The cells were permeabilized with digitonin before fixation.
(G) Immunofluorescence of U2OS co-expressing Q103-HTT-BFP and mCherry or HA-CCT2 with anti-HA and LAMP2 antibodies. The cells were permeabilized with digitonin before fixation.
(H) Quantification of LAMP2 around Q103 IB (mean±SEM) as shown in (F). P values are indicated (two-tailed t test, >50 cells from three independent experiments).
(I, K, M) Turnover of Q103-HTT in CHX chase assay in U2OS (I), N2A (K) and primary striatal neuron (M) with or without HA-CCT2 co-expression.
(J, L, N) Quantification of normalized Q103-HTT (mean±SEM) in (I, K, M). P values are indicated (two-way ANOVA, three independent experiments).

FIG. 4 shows CCT2 promotes autophagic clearance of mutant Tau and SOD1, wherein, (A) Immunofluorescence of U2OS co-expressing GFP-Tau P301L with control or HA-CCT2 using anti-HA and LC3 antibodies. The cells were permeabilized with digitonin before fixation. Arrows point to the triple colocalization of Tau, CCT2 and LC3.
(B) Quantification of Tau-LC3 colocalization and Tau-CCT2-LC3 triple colocalization (mean±SEM) in (A). P values are indicated (two-tailed t test, >50 cells from three independent experiments).
(C) Turnover of GFP-Tau P301L in CHX chase assay with or without HA-CCT2 expression in U2OS.
(D) Quantification of normalized Tau P301L (mean±SEM) in (C). P values are indicated (two-way ANOVA, three independent experiments).
(E) Turnover of SOD1 G93A-GFP in CHX chase assay with or without HA-CCT2 expression in U2OS.
(F) Quantification of normalized SOD1 G93A (mean±SEM) in (E). P values are indicated (two-way ANOVA, three independent experiments).
(G) Turnover of digitonin-insoluble Q103-HTT in CHX chase assay in U2OS cells. For the determination of digitonin-insoluble Q103-HTT, the cells were permeabilized with 40 μg/ml digitonin on ice to release soluble proteins before immunoblot analysis.
(H) Quantification of normalized Q103-HTT (mean±SEM) in (G). P values are indicated (two-way ANOVA, three independent experiments).
(I) Accumulation of exogenous HA-CCT2 in HEK293T. 24 h after HA-CCT2 expression, the cells were treated with or without 200 ng/ml BafA1 for 4 h. The numbers indicate relative enrichment of HA-CCT2. The data are representative of three independent experiments.
(J) Accumulation of endogenous CCT2 in HEK293T cells and autophagosomes. HEK293T cells were transfected with Q103-HTT-GFP. 24 h after transfection, the cells were treated with or without 200 ng/ml BafA1 for 8h and membrane fractionation was performed to isolate autophagosome fractions (F-AG) as shown in (K). The numbers indicate relative enrichment of CCT2 in the indicated fractions. The data are representative of three independent experiments.
(K) HEK293T cells were transfected with Q103-HTT-GFP and treated with or without 200 ng/ml BafA1 for 8 h. The immunoblot shows the distribution of endogenous CCT2 in the OptiPrep gradient fractions 1-10 from experiments shown in FIG. 2J. The data are representative of three independent experiments.

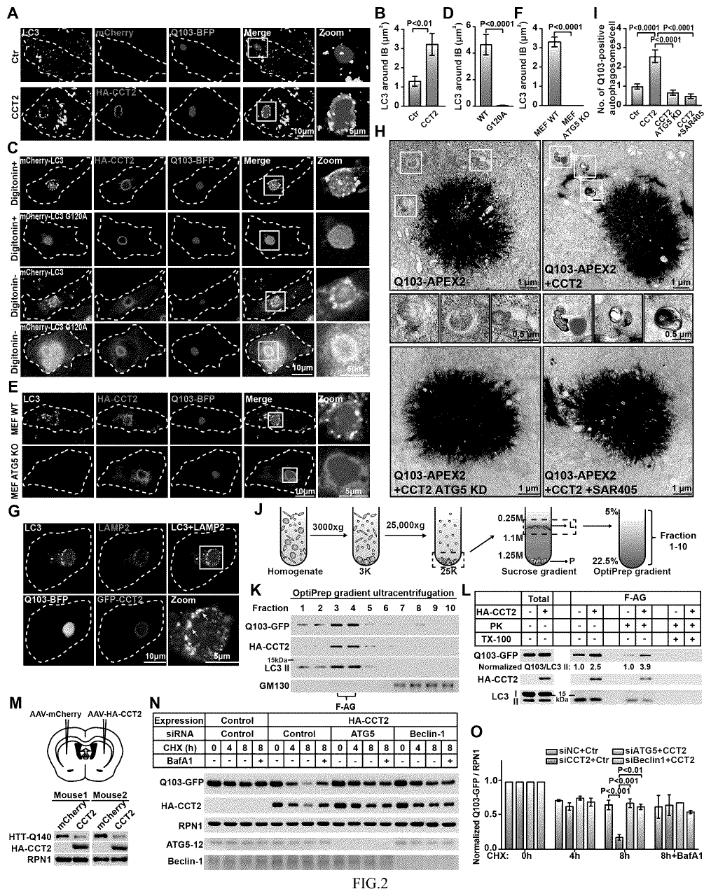

FIG. 5 shows CCT2 interacts with ATG8s, wherein, (A) Co-IP analysis of HA-CCT2 with T7-ATG8s in HEK293T. The data are representative of three independent experiments.
(B) Co-IP analysis of GFP-tagged CCT2 variants (FL, full length CCT2; D1, aa1-216; D2, aa217-368 of CCT2; D3, aa369-535 of CCT2) with T7-LC3C in HEK293T. The data are representative of three independent experiments.
(C) Peptides (P1-P8) from CCT2 D3 and peptide 7 mutant (mVL (I) L) were immobilized to agarose beads followed by analysis of direct T7-LC3C interaction in an in vitro pull-down assay. The data are representative of three independent experiments.
(D) Co-IP analysis of HA-CCT2 variants (WT, wild type CCT2; Apep7, CCT2 deleted peptide 7; VLL and VIL, CCT2 indicated site mutated to alanine) with T7-LC3C in HEK293T. The data are representative of three independent experiments.
(E) In vitro pull-down of purified GST-CCT2s and His-T7-LC3C proteins. The data are representative of three independent experiments.
(F) Immunofluorescence of U2OS co-expressing Q103-HTT-BFP and mCherry or HA-CCT2 variants with anti-HA and LC3 antibodies. The cells were permeabilized with digitonin before fixation.
(G) Quantification of LC3 around Q103-HTT IB (mean±SEM) as shown in (F). P values are indicated (one-way ANOVA, >50 cells from three independent experiments).
(H) Turnover of digitonin-insoluble Q103-HTT in CHX chase assay in WT or CCT2 knockdown U2OS with or without HA-CCT2 variants re-expression. The cells were permeabilized with digitonin before immunoblot analysis. LE, long exposure; SE, short exposure.
(I) Quantification of normalized Q103-HTT (mean±SEM) in (H). P values are indicated (two-wayANOVA, two independent experiments).
(J) Sequence showing the location of the two mutations in CCT2 (Blue). The VLIR motif VIL is highlighted in red.
(K) Co-IP analysis of HA-CCT2 variants with T7-LC3C in HEK293T. The data are representative of three independent experiments.
(L) Immunofluorescence of CCT2 knockdown U2OS co-expressing Q103-HTT-BFP and HA-CCT2 variants with anti-HA and LC3 antibodies. The cells were permeabilized with digitonin before fixation.
(M) Quantification of LC3 around IBs (mean±SEM) as shown in (L). P values are indicated (two-tailed t test, >50 cells from three independent experiments).
(N) Turnover of Q103-HTT in the CHX chase assay in CCT2 knockdown U2OS with HA-CCT2 variants re-expression.
(O) Quantification of normalized Q103-HTT (mean±SEM) in (N). P values are indicated (two-way ANOVA, three independent experiments).

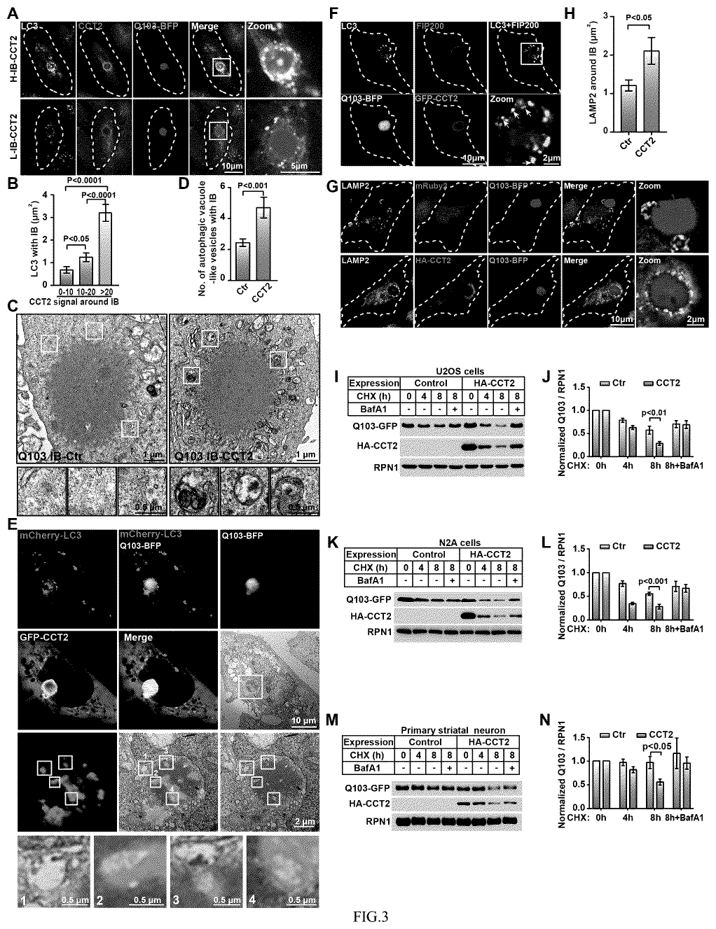

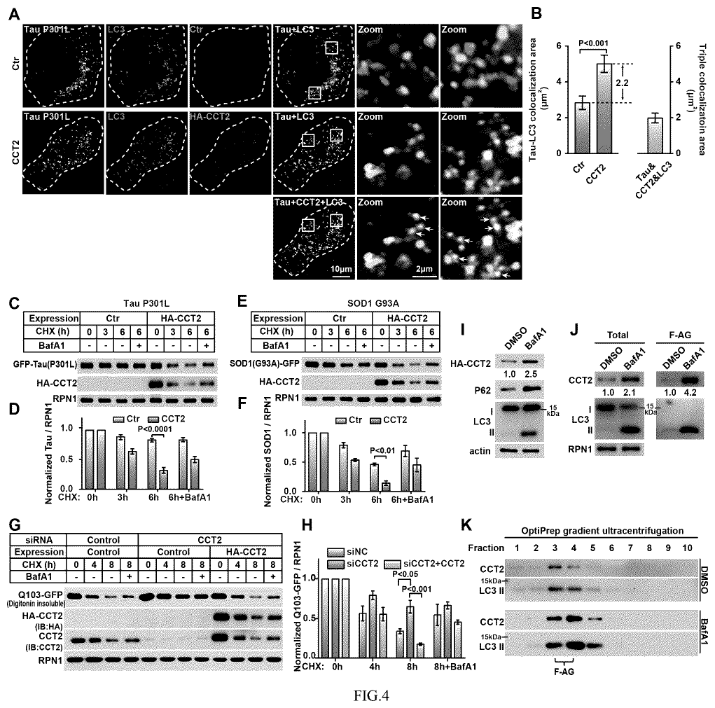

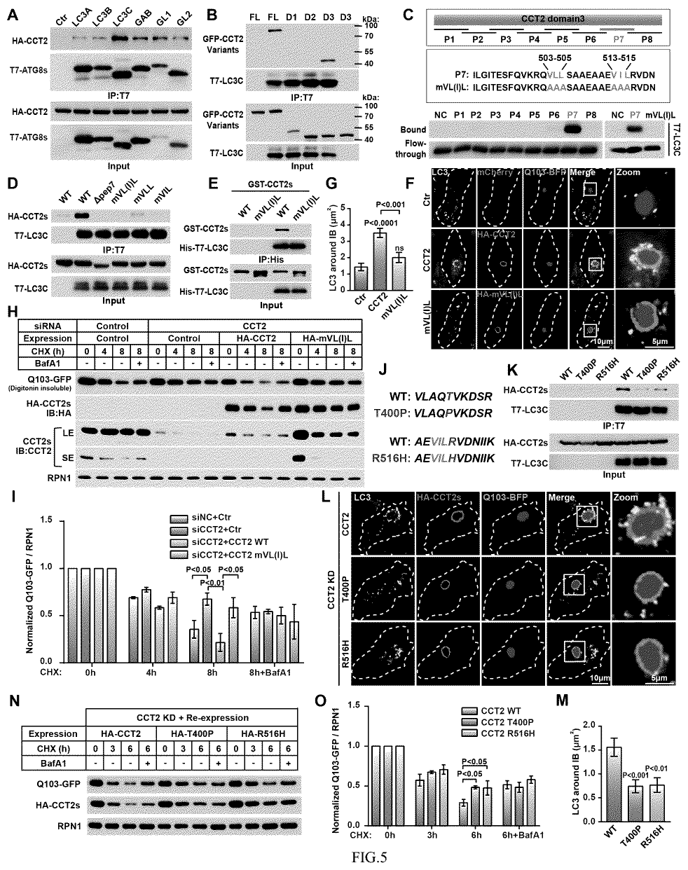

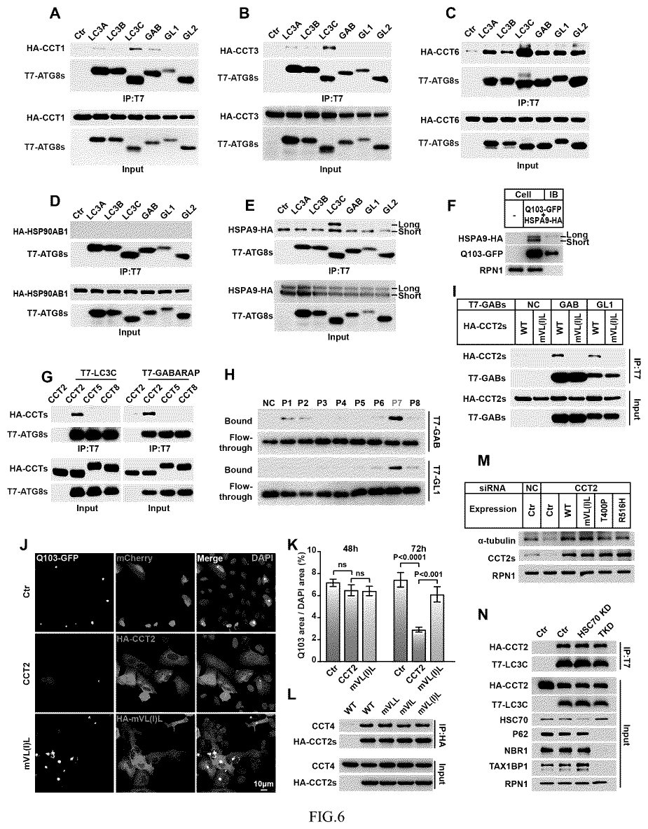

FIG. 6 shows interaction of CCT2 with ATG8s and the role in polyQ-HTT degradation, wherein, (A-E) Co-IP analysis of CCT1 (A), CCT3 (B), CCT6 (C), HSP90AB1 (D), or HSPA9 (E) with T7-LC3C in HEK293T. The data are representative of three independent experiments.
(F) HEK293T was transfected with or without Q103-GFP and HSPA9-HA. Total cell or Q103-HTT IB was collected for immunoblot to determine the form of HSPA9-HA. The data are representative of three independent experiments.
(G) Co-IP analysis of HA-CCT2, CCT5, and CCT8 with T7-LC3C/GABARAP in HEK293T. The data are representative of three independent experiments.
(H) Peptides (P1-P8) from CCT2 D3 were immobilized to agarose beads using the AminoLink Coupling Resin. The interaction of the peptides with T7-GABARAP or T7-GABARAPL1 proteins was analyzed by in vitro pull-down. The data are representative of three independent experiments.
(I) Co-IP analysis of the HA-CCT2 variants with T7-GABARAP or T7-GABARAPL1 in HEK293T. The data are representative of three independent experiments.
(J) Immunofluorescence of U2OS co-expressing Q103-HTT-GFP and mCherry or HA-CCT2 variants for 72h.
(K) Quantification of Q103-HTT-GFP area/DAPI area (mean±SEM) as shown in
(J). 48 h represents IB accumulation stage and 72 h represents clearance stage. P values are indicated (two-tailed t test, >20 views from three independent experiments).
(L) Co-IP analysis of HA-CCT2 variants with endogenous CCT4 in HEK293T. The data are representative of three independent experiments.
(M) Immunoblot of α-tubulin after CCT2 variants re-expression in CCT2 knockdown U2OS. The data are representative of three independent experiments.
(N) Co-IP analysis of HA-CCT2 with T7-LC3C in HEK293T with indicated gene knockdown. The data are representative of three independent experiments.

FIG. 7 shows CCT2 functions independent of cargo ubiquitination in aggrephagy, wherein, (A-C) Co-IP analyses of HA-CCT2 with the indicated GFP-tagged aggregation-prone proteins including Q103-HTT (A), Tau P301L (B), and SOD1 G93A (C) in HEK293T. The data are representative of three independent experiments. Asterisks indicate degradation bands.
(D) Co-IP analysis of Q103-HTT-BFP with the GFP-tagged CCT2 variants in HEK293T using Protein A/G agarose and BFP antibodies. The data are representative of three independent experiments.
(E) In vitro pull-down showing the binding of GST-CCT2 or P62 to the purified Ubx8. The data are representative of three independent experiments. The asterisk indicates a degradation band.
(F) In vitro pull-down showing the interaction of GST-CCT2 or P62 with the polyubiquitin chain from HEK293T cell lysates. The data are representative of three independent experiments. Asterisks indicate degradation bands.
(G) Duolink PLA assay showing the interaction of Q103-HTT-T7 or Q103KR-HTT-T7 withHA-CCT2 in U2OS.
(H) Quantification of the Duolink PLA signal (mean±SEM) as shown in (G). P values are indicated (two-tailed t test, >50 cells from three independent experiments).
(I) Filter trap assay showing the turnover of Q103-HTT-T7 and Q103KR-HTT-T7 in CHX chase assay in control or HA-CCT2 expression U2OS. Equal amount of cell lysates were loaded.
(J-K) Quantification of normalized Q103-HTT-T7 (J) and Q103KR-HTT-T7 (K) (mean±SEM) in (I). P values are indicated (two-way ANOVA, two independent experiments).

FIG. 8 shows CCT2 acts independent of P62, NBR1, TAXIBP1, and CMA, wherein, (A) Immunofluorescence of WT or TKD (triple knockdown of P62, NBR1, and TAX1BP1) U2OS co-expressing Q103-HTT-BFP and mCherry or HA-CCT2 with anti-HA and LC3 antibodies. The cells were permeabilized with digitonin before fixation.
(B) Quantification of LC3 around Q103-HTT IBs (mean±SEM) as shown in (A). P values are indicated (two-tailed t test, >50 cells from three independent experiments).
(C) Turnover of Q103-HTT in CHX chase assay with or without HA-CCT2 expression in WT or TKD U2OS.
(D) Quantification of normalized Q103-HTT (mean±SEM) in (C). P values are indicated (two-way ANOVA, three independent experiments).
(E) Immunofluorescence of WT or HSC70 knockdown U2OS co-expressing Q103-HTT-BFP and mCherry or HA-CCT2 with anti-HA and LC3 antibodies. The cells were permeabilized with digitonin before fixation.
(F) Quantification of LC3 around Q103-HTT IB (mean±SEM) as shown in (E). P values are indicated (two-tailed t test, >50 cells from three independent experiments).
(G) Turnover of Q103-HTT in CHX chase assay with or without HA-CCT2 expression in WT or HSC70 KD U2OS cells.
(H) Quantification of normalized Q103-HTT (mean±SEM) in (G). P values are indicated (two-way ANOVA, three independent experiments).

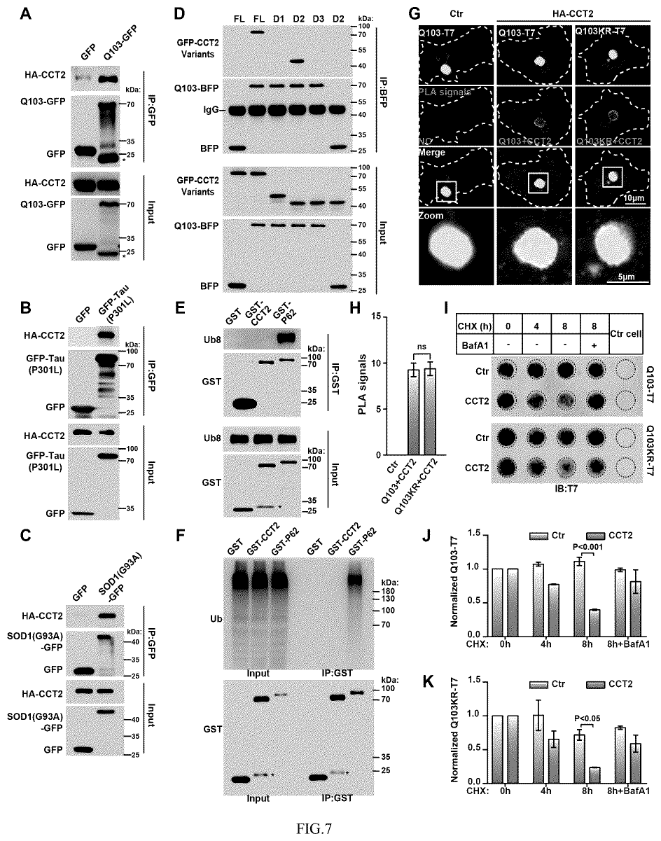

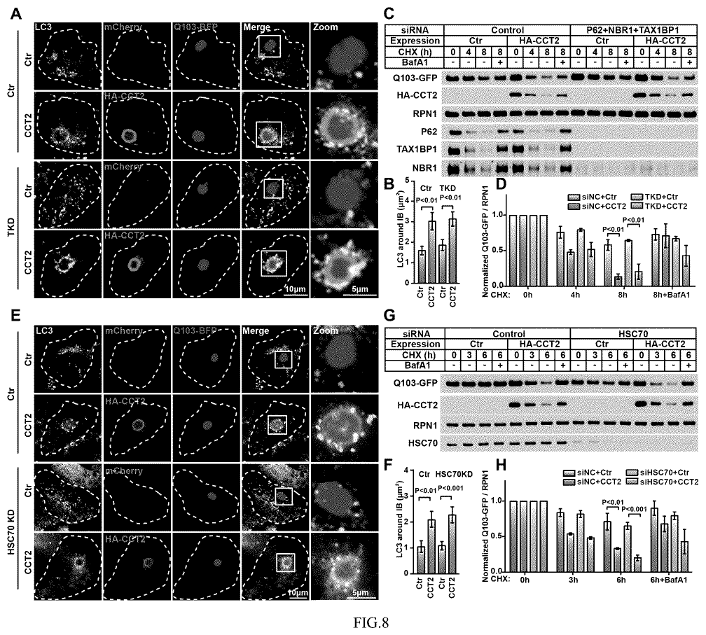

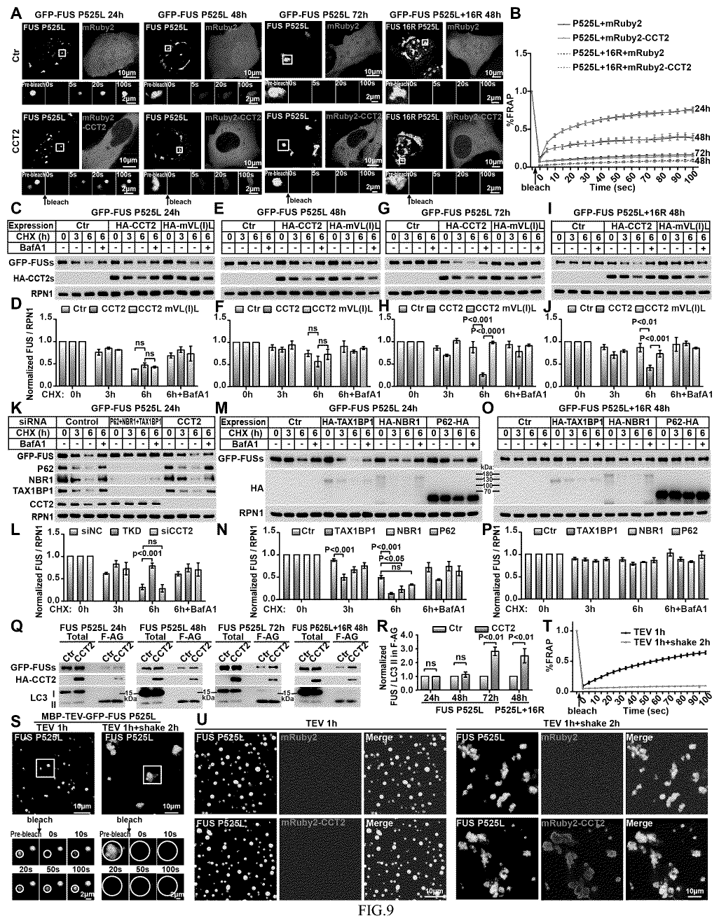

FIG. 9 shows CCT2 promotes the clearance of protein condensates with little liquidity, wherein, (A) The indicated GFP-FUS mutants, and mRuby2 or mRuby2-CCT2 were co-expressed in U2OS for 24 h, 48h and 72h. FRAP analysis was performed at the indicated time points.
(B) Quantification of the normalized GFP-FUS mutants fluorescence signal (mean±SEM) in (A) (>30 cells from three independent experiments).
(C, E, G, I) Turnover of the indicated GFP-FUS mutants (P525L (C, E, G) and P252L+16R (I)) in CHX chase assay with or without HA-CCT2 variants expression in U2OS at 24h (C), 48h (E, I) and 72h (G) after transfection.
(D, F, H, J) Quantification of the normalized GFP-FUS mutants (mean±SEM) in (D to C, F to E, G to H, J to I). P values are indicated (two-way ANOVA, three independent experiments).
(K) Turnover of GFP-FUS P525L in CHX chase assay in WT, TKD (P62, NBR1, and TAX1BP1) or CCT2 KD U2OS cells at 24 h after transfection.
(L) Quantification of the normalized GFP-FUS P525L (mean±SEM) in (K). P values are indicated (two-way ANOVA, three independent experiments).
(M) Turnover of GFP-FUS P525L in CHX chase assay with or without TAX1BP1, NBR1, or P62 expression in U2OS at 24 h after transfection.
(N) Quantification of the normalized GFP-FUS P525L (mean±SEM) in (M). P values are indicated (two-way ANOVA, three independent experiments).
(O) Turnover of GFP-FUS P525L+16R in CHX chase assay with or without TAX1BP1, NBR1, or P62 expression in U2OS at 48h after transfection.
(P) Quantification of the normalized GFP-FUS P525L+16R (mean±SEM) in (O). P values are indicated (two-way ANOVA, three independent experiments).
(Q) The GFP-FUS mutants were expressed with or without HA-CCT2 in HEK293T for 24 h, 48h and 72h as indicated. The autophagosome fractions (F-AG) were isolated and the indicated proteins were determined.
(R) Quantification of the normalized GFP-FUS mutants (mean±SEM) in F-AG as shown in (Q). P values are indicated (two-way ANOVA, two independent experiments).
(S) In vitro FUS P525L phase separation and aggregation. FRAP showed the liquidity of FUS granules.
(T) Quantification of the normalized fluorescence signal of FUS granules (mean±SEM) in(S) (>50 granules from three independent experiments).
(U) The recruitment of CCT2 to the liquid or solid FUS P525L granules shown in(S).

FIG. 10 shows CCT2 acts independent of the TRIC complex in aggrephagy, wherein, (A) Immunofluorescence of WT or CCT4 KD U2OS co-expressing Q103-HTT-BFP and mCherry or HA-CCT2 with anti-HA and LC3 antibodies. The cells were permeabilized with digitonin before fixation.
(B) Turnover of Q103-HTT in CHX chase assay with or without HA-CCT2 expression in WT or CCT4 KD U20S.
(C) Quantification of normalized Q103-HTT (mean±SEM) in (B). P values are indicated (two-way ANOVA, three independent experiments).
(D) Duolink PLA assay showing the interaction between V5-CCT2 and HA-CCTs (CCT1&3˜8) in the presence of Q10-HTT or Q103-HTT.
(E) Quantification of the Duolink PLA signal (mean±SEM) as shown in (D). P values are indicated (two-tailed t test, >50 cells from three independent experiments).
(F) Immunoblot of total HEK293T cell lysates after incubating with 1.6 μM purified CFP or Q45-CFP proteins for 2 h at 4° C.
(G) Gel-filtration analysis of CCTs in HEK293T cell lysates in (F). The numbers indicate percentage of CCT2 in the monomer fraction. The data are representative of three independent experiments.
(H) Structure of TRIC (PDB: 7LUM) and the location of the LC3-interaction motifs VLL and VIL on CCT2. The structure model was created by PyMOL.
(I) In vitro pull-down assay showing the interaction of His-T7-LC3C with complex (F13) or monomer (F17) form of CCT2 from (G). The data are representative of three independent experiments.
(J) Gel-filtration showing the form of exogenously expressed V5-CCT2 with or without other CCTs (HA-CCT1&3˜8). The data are representative of three independent experiments.
(K) Immunofluorescence of U2OS co-expressing Q103-HTT-BFP, V5-CCT2 with or without HA-CCTs (CCT1&3˜8) as indicated with anti-V5, HA and LC3 antibodies.
(L) Quantification of LC3 around Q103-HTT IB (mean±SEM) as shown in (K). P values are indicated (one-way ANOVA, >50 cells from three independent experiments).
(M) Turnover of Q103-HTT in CHX chase assay with or without V5-CCT2, HA-CCTs (CCT1&3˜8) expression as indicated in U2OS.
(N) Quantification of normalized Q103-HTT (mean±SEM) in (M). P values are indicated (two-way ANOVA, two independent experiments).

FIG. 11 shows CCT2 acts independently of the TRIC complex in aggrephagy, wherein, (A) Immunoblot of α-tubulin in U2OS transfected with siRNA against control, CCT4 or CCT5. The data are representative of three independent experiments.
(B) Quantification of LC3 around Q103-HTT IBs (mean±SEM) as shown in (C and FIG. 7A). P values are indicated (two-tailed t test, >50 cells from three independent experiments).
(C) Immunofluorescence of WT or CCT5 KD U2OS co-expressing Q103-HTT-BFP with mCherry or HA-CCT2 using anti-HA and LC3 antibodies. The cells were permeabilized with digitonin before fixation.
(D) Turnover of Q103-HTT in CHX chase assay with or without HA-CCT2 expression in WT or CCT5 KD U20S.
(E) Quantification of normalized Q103-HTT (mean±SEM) in (D). P values are indicated (two-way ANOVA, three independent experiments).
(F) Duolink PLA assay showing the interaction between V5-CCT2 and T7-LC3C with or without other CCTs (HA-CCT1&3˜8) expression. GFP was co-expressed to mark successfully transfected cells. Duolink PLA assay was performed with equal conditions, and the Duolink PLA signals were acquired with equal settings between each group.
(G) Quantification of the Duolink PLA signal (mean±SEM) as shown in (F). P values are indicated (two-tailed t test, >50 cells from three independent experiments).
(H) Co-IP analysis of HA-CCT2 variants with T7-CCT4 in HEK293T. The data are representative of three independent experiments.
(I) Gel-filtration showing the form of exogenously expressed V5-CCT2 WT or T400P and HA-CCTs (CCT1&3˜8) from HEK293T cell lysates after incubating with 1.6 μM purified CFP or Q45-CFP proteins. The numbers indicate the percentage of CCT2 in the monomer fraction. The data are representative of three independent experiments.
(J) A model for the functional switch of CCT2 from a chaperonin subunit to an autophagy receptor.

FIG. 12 shows that overexpression of CCT2 alleviates neurodegenerative phenotypes at neuronal, histopathological and behavioral level, wherein, (A, C) Representative images of striatal (A) and hippocampal neurons (B) and dendritic segments (zoom) labeled by triple fluorescence of aggregation-prone proteins (Q103-GFP, Tau-GFP), CCT2 (WT and R516H), and synapsin (synapse), scale bar, 30 μm (upper panel), 5 μm (lower panel).
(B, D) Quantification of synapse number. * P<0.05, ** P<0.01, *** P<0.001 (two-tailed t test, >50 cells from three independent experiments).
(E) Q140 KI het cohorts injected with AAV2-GFP/HA-CCT2 WT/HA-CCT2 R516H; scale bar, 20 μm. HTT inclusions (mEM48), purple; GFP/HA, green; DAPI, blue.
(F) The representative open field activity tracks show the 5 experimental groups used in behavioral analyses.
(G) Open field testing for total distance traveled was performed at 8 weeks of age on AAV-mCherry/CCT2 WT/CCT2 R516H-treated R6/2 male mice. * P<0.05 (two-tailed t test).

FIG. 13 shows CCT1/3/6 and CCT2 fusion proteins promote clearance of solid aggregates, wherein, (A) Turnover of GFP-FUS P525L+16R in CHX chase assay with or without CCT1/3/6 expression in U2OS at 48h after transfection.
(B) Turnover of GFP-FUS P525L+16R in CHX chase assay with or without CCT2 D2-V5-D3 expression in U2OS at 48h after transfection.
(C) Co-IP showing the interaction of CCT2 D2-P7 with LC3C.
(D) Turnover of GFP-FUS P525L+16R in CHX chase assay with or without CCT2 D2-P7 expression in U2OS at 48h after transfection.
(E) Turnover of GFP-Tau (P301L) in CHX chase assay with or without CCT2 D2-P7 expression in U2OS at 48h after transfection.

### DETAILED DESCRIPTION OF THE EMBODIMENTS

The aforementioned features and advantages of the invention as well as additional features and advantages thereof will be more clearly understood hereafter as a result of a detailed description of the following embodiments when taken conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless otherwise specified, “chaperone” mentioned in this application refers to a group of proteins that have functional similarity and assist in protein folding. They are proteins that have the ability to prevent non-specific aggregation by binding to non-native proteins.

According to an embodiment of the present invention, chaperone subunit CCT2 has the amino acid sequence shown in SEQ ID NO:7. The P7 Peptide of CCT2 described in this application is the peptide shown by amino acids 490 ˜519 in SEQ ID NO:7.

(SEQ ID NO: 7)
MASLSLAPVNIFKAGADEERAETARLTSFIGAIAIGDLVKSTLGPKGMDK
ILLSSGRDASLMVTNDGATILKNIGVDNPAAKVLVDMSRVQDDEVGDGTT
SVTVLAAELLREAESLIAKKIHPQTIIAGWREATKAAREALLSSAVDHGS
DEVKFRQDLMNIAGTTLSSKLLTHHKDHFTKLAVEAVLRLKGSGNLEAIH
IIKKLGGSLADSYLDEGFLLDKKIGVNQPKRIENAKILIANTGMDTDKIK
IFGSRVRVDSTAKVAEIEHAEKEKMKEKVERILKHGINCFINRQLIYNYP
EQLFGAAGVMAIEHADFAGVERLALVTGGEIASTFDHPELVKLGSCKLIE
EVMIGEDKLIHFSGVALGEACTIVLRGATQQILDEAERSLHDALCVLAQT
VKDSRTVYGGGCSEMLMAHAVTQLANRTPGKEAVAMESYAKALRMLPTII
ADNAGYDSADLVAQLRAAHSEGNTTAGLDMREGTIGDMAILGITESFQVK
RQVLLSAAEAAEVILRVDNIIKAAPRKRVPDHHPC.

According to an embodiment of the present invention, chaperone subunit CCT6 has the amino acid sequence shown in SEQ ID NO:8.

(SEQ ID NO: 8)
MAAVKTLNPKAEVARAQAALAVNISAARGLQDVLRTNLGPKGTMKMLVSG
AGDIKLTKDGNVLLHEMQIQHPTASLIAKVATAQDDITGDGTTSNVLIIG
ELLKQADLYISEGLHPRIITEGFEAAKEKALQFLEEVKVSREMDRETLID
VARTSLRTKVHAELADVLTEAVVDSILAIKKQDEPIDLFMIEIMEMKHKS
ETDTSLIRGLVLDHGARHPDMKKRVEDAYILTCNVSLEYEKTEVNSGFFY
KSAEEREKLVKAERKFIEDRVKKIIELKRKVCGDSDKGFVVINQKGIDPF
SLDALSKEGIVALRRAKRRNMERLTLACGGVALNSFDDLSPDCLGHAGLV
YEYTLGEEKFTFIEKCNNPRSVTLLIKGPNKHTLTQIKDAVRDGLRAVKN
AIDDGCVVPGAGAVEVAMAEALIKHKPSVKGRAQLGVQAFADALLIIPKV
LAQNSGFDLQETLVKIQAEHSESGQLVGVDLNTGEPMVAAEVGVWDNYCV
KKQLLHSCTVIATNILLVDEIMRAGMSSLKG.

According to an embodiment of the present invention, chaperone subunit CCT1 has the amino acid sequence shown in SEQ ID NO:9.

(SEQ ID NO: 9)
MEGPLSVFGDRSTGETIRSQNVMAAASIANIVKSSLGPVGLDKMLVDDIG
DVTITNDGATILKLLEVEHPAAKVLCELADLQDKEVGDGTTSVVIIAAEL
LKNADELVKQKIHPTSVISGYRLACKEAVRYINENLIVNTDELGRDCLIN
AAKTSMSSKIIGINGDFFANMVVDAVLAIKYTDIRGQPRYPVNSVNILKA
HGRSQMESMLISGYALNCVVGSQGMPKRIVNAKIACLDFSLQKTKMKLGV
QVVITDPEKLDQIRQRESDITKERIQKILATGANVILTTGGIDDMCLKYF
VEAGAMAVRRVLKRDLKRIAKASGATILSTLANLEGEETFEAAMLGQAEE
VVQERICDDELILIKNTKARTSASIILRGANDFMCDEMERSLHDALCVVK
RVLESKSVVPGGGAVEAALSIYLENYATSMGSREQLAIAEFARSLLVIPN
TLAVNAAQDSTDLVAKLRAFHNEAQVNPERKNLKWIGLDLSNGKPRDNKQ
AGVFEPTIVKVKSLKFATEAAITILRIDDLIKLHPESKDDKHGSYEDAVH
SGALND.

According to an embodiment of the present invention, chaperone subunit CCT3 has the amino acid sequence shown in SEQ ID NO:10.

(SEQ ID NO: 10)
MMGHRPVLVLSQNTKRESGRKVQSGNINAAKTIADIIRTCLGPKSMMKML
LDPMGGIVMTNDGNAILREIQVQHPAAKSMIEISRTQDEEVGDGTTSVII
LAGEMLSVAEHFLEQQMHPTVVISAYRKALDDMISTLKKISIPVDISDSD
MMLNIINSSITTKAISRWSSLACNIALDAVKMVQFEENGRKEIDIKKYAR
VEKIPGGIIEDSCVLRGVMINKDVTHPRMRRYIKNPRIVLLDSSLEYKKG
ESQTDIEITREEDFTRILQMEEEYIQQLCEDIIQLKPDVVITEKGISDLA
QHYLMRANITAIRRVRKTDNNRIARACGARIVSRPEELREDDVGTGAGLL
EIKKIGDEYFTFITDCKDPKACTILLRGASKEILSEVERNLQDAMQVCRN
VLLDPQLVPGGGASEMAVAHALTEKSKAMTGVEQWPYRAVAQALEVIPRT
LIQNCGASTIRLLTSLRAKHTQENCETWGVNGETGTLVDMKELGIWEPLA
VKLQTYKTAVETAVLLLRIDDIVSGHKKKGDDQSRQGGAPDAGQE.

According to an embodiment of the present invention, chaperone HSPA9 has the amino acid sequence shown in SEQ ID NO:11.

(SEQ ID NO: 11)
MISASRAAAARLVGAAASRGPTAARHQDSWNGLSHEAFRLVSRRDYASEA
IKGAVVGIDLGTTNSCVAVMEGKQAKVLENAEGARTTPSVVAFTADGERL
VGMPAKRQAVTNPNNTFYATKRLIGRRYDDPEVQKDIKNVPFKIVRASNG
DAWVEAHGKLYSPSQIGAFVLMKMKETAENYLGHTAKNAVITVPAYENDS
QRQATKDAGQISGLNVLRVINEPTAAALAYGLDKSEDKVIAVYDLGGGTF
DISILEIQKGVFEVKSTNGDTFLGGEDFDQALLRHIVKEFKRETGVDLTK
DNMALQRVREAAEKAKCELSSSVQTDINLPYLTMDSSGPKHLNMKLTRAQ
FEGIVTDLIRRTIAPCQKAMQDAEVSKSDIGEVILVGGMTRMPKVQQTVQ
DLFGRAPSKAVNPDEAVAIGAAIQGGVLAGDVTDVLLLDVTPLSLGIETL
GGVFTKLINRNTTIPTKKSQVFSTAADGQTQVEIKVCQGEREMAGDNKLL
GQFTLIGIPPAPRGVPQIEVTFDIDANGIVHVSAKDKGTGREQQIVIQSS
GGLSKDDIENMVKNAEKYAEEDRRKKERVEAVNMAEGIIHDTETKMEEFK
DQLPADECNKLKEEISKMRELLARKDSETGENIRQAASSLQQASLKLFEM
AYKKMASEREGSGSSGTGEQKEDQKEEKQ.

According to an embodiment of the present invention, chaperone HSP90AB1 has the amino acid sequence shown in SEQ ID NO:12.

(SEQ ID NO: 12)
MPEEVHHGEEEVETFAFQAEIAQLMSLIINTFYSNKEIFLRELISNASDA
LDKIRYESLTDPSKLDSGKELKIDIIPNPQERTLTLVDTGIGMTKADLIN
NLGTIAKSGTKAFMEALQAGADISMIGQFGVGFYSAYLVAEKVVVITKHN
DDEQYAWESSAGGSFTVRADHGEPIGRGTKVILHLKEDQTEYLEERRVKE
VVKKHSQFIGYPITLYLEKEREKEISDDEAEEEKGEKEEEDKDDEEKPKI
EDVGSDEEDDSGKDKKKKTKKIKEKYIDQEELNKTKPIWTRNPDDITQEE
YGEFYKSLTNDWEDHLAVKHFSVEGQLEFRALLFIPRRAPFDLFENKKKK
NNIKLYVRRVFIMDSCDELIPEYLNFIRGVVDSEDLPLNISREMLQQSKI
LKVIRKNIVKKCLELFSELAEDKENYKKFYEAFSKNLKLGIHEDSTNRRR
LSELLRYHTSQSGDEMTSLSEYVSRMKETQKSIYYITGESKEQVANSAFV
ERVRKRGFEVVYMTEPIDEYCVQQLKEFDGKSLVSVTKEGLELPEDEEEK
KKMEESKAKFENLCKLMKEILDKKVEKVTISNRLVSSPCCIVTSTYGWTA
NMERIMKAQALRDNSTMGYMMAKKHLEINPDHPIVETLRQKAEADKNDKA
VKDLVVLLFETALLSSGFSLEDPQTHSNRIYRMIKLGLGIDEDEVAAEEP
NAAVPDEIPPLEGDEDASRMEEVD.

According to an embodiment of the present invention, the fusion protein comprising D2 domain of CCT2 and D3 domain of CCT2 (CCT2 D2-V5-D3) has the amino acid sequence shown in SEQ ID NO:13.

(SEQ ID NO: 13)
MGFLLDKKIGVNQPKRIENAKILIANTGMDTDKIKIFGSRVRVDSTAKVA
EIEHAEKEKMKEKVERILKHGINCFINRQLIYNYPEQLFGAAGVMAIEHA
DFAGVERLALVTGGEIASTFDHPELVKLGSCKLIEEVMIGEDKLIHFSGV
ALGGKPIPNPLLGLDSTEACTIVLRGATQQILDEAERSLHDALCVLAQTV
KDSRTVYGGGCSEMLMAHAVTQLANRTPGKEAVAMESYAKALRMLPTIIA
DNAGYDSADLVAQLRAAHSEGNTTAGLDMREGTIGDMAILGITESFQVKR
QVLLSAAEAAEVILRVDNIIKAAPRKRVPDHHPC.

According to an embodiment of the present invention, the fusion protein comprising D2 domain of CCT2 and P7 peptide of CCT2 (CCT2 D2-P7) has the amino acid sequence shown in SEQ ID NO:14.

(SEQ ID NO: 14)
MGFLLDKKIGVNQPKRIENAKILIANTGMDTDKIKIFGSRVRVDSTAKVA
EIEHAEKEKMKEKVERILKHGINCFINRQLIYNYPEQLFGAAGVMAIEHA
DFAGVERLALVTGGEIASTFDHPELVKLGSCKLIEEVMIGEDKLIHFSGV
ALGGSGGILGITESFQVKRQVLLSAAEAAEVILRVDN.

According to an embodiment of the present invention, the CCT2 D2-V5-D3 coding nucleic acid has the nucleotide sequence shown in SEQ ID No: 15.

(SEQ ID NO: 15)
ATGGGCTTCCTGTTGGATAAAAAAATTGGAGTAAATCAACCAAAACGAAT
TGAAAATGCTAAAATTCTTATTGCAAATACTGGTATGGATACAGACAAAA
TAAAGATATTTGGTTCCCGGGTAAGAGTTGACTCTACAGCAAAGGTTGCA
GAAATAGAACATGCGGAAAAGGAAAAAATGAAGGAGAAAGTTGAACGTAT
TCTTAAGCATGGAATAAATTGCTTTATTAACAGGCAATTAATTTATAATT
ATCCTGAACAGCTCTTTGGTGCTGCTGGTGTCATGGCTATTGAGCATGCA
GATTTTGCAGGTGTGGAACGCCTAGCTCTTGTCACAGGTGGTGAAATTGC
CTCTACCTTTGATCACCCAGAACTGGTGAAGCTTGGAAGTTGCAAACTTA
TCGAGGAAGTCATGATTGGAGAAGACAAACTCATTCACTTTTCTGGGGTT
GCCCTTGGTGGTAAGCCTATCCCTAACCCTCTCCTCGGTCTCGATTCTAC
GGAGGCTTGTACCATTGTTTTGCGTGGTGCCACTCAACAAATTTTAGATG
AAGCAGAAAGATCATTGCATGATGCTCTTTGTGTTCTTGCGCAAACTGTA
AAGGACTCTAGAACAGTTTATGGAGGAGGCTGTTCTGAGATGTTGATGGC
TCATGCTGTGACACAGCTTGCCAATAGAACACCAGGCAAAGAAGCTGTTG
CAATGGAGTCTTATGCTAAAGCACTGAGAATGTTGCCAACCATCATAGCT
GACAATGCAGGCTATGACAGTGCAGACCTGGTGGCACAGCTCAGGGCTGC
TCACAGTGAAGGCAATACCACTGCTGGATTGGATATGAGGGAAGGCACCA
TTGGAGATATGGCTATCCTGGGTATAACAGAAAGTTTTCAAGTGAAGCGA
CAGGTTCTTCTGAGTGCAGCTGAAGCAGCAGAGGTGATTCTGCGTGTGGA
CAACATCATCAAAGCGGCACCCAGGAAACGTGTCCCTGATCACCACCCCT
GTTAG.

According to an embodiment of the present invention, the CCT2 D2-P7 coding nucleic acid has the nucleotide sequence shown in SEQ ID No: 16.

(SEQ ID NO: 16)
ATGGGCTTCCTGTTGGATAAAAAAATTGGAGTAAATCAACCAAAACGAAT
TGAAAATGCTAAAATTCTTATTGCAAATACTGGTATGGATACAGACAAAA
TAAAGATATTTGGTTCCCGGGTAAGAGTTGACTCTACAGCAAAGGTTGCA
GAAATAGAACATGCGGAAAAGGAAAAAATGAAGGAGAAAGTTGAACGTAT
TCTTAAGCATGGAATAAATTGCTTTATTAACAGGCAATTAATTTATAATT
ATCCTGAACAGCTCTTTGGTGCTGCTGGTGTCATGGCTATTGAGCATGCA
GATTTTGCAGGTGTGGAACGCCTAGCTCTTGTCACAGGTGGTGAAATTGC
CTCTACCTTTGATCACCCAGAACTGGTGAAGCTTGGAAGTTGCAAACTTA
TCGAGGAAGTCATGATTGGAGAAGACAAACTCATTCACTTTTCTGGGGTT
GCCCTTGGTGGAAGTGGTGGAATCCTGGGTATAACAGAAAGTTTTCAAGT
GAAGCGACAGGTTCTTCTGAGTGCAGCTGAAGCAGCAGAGGTGATTCTGC
GTGTGGACAACTGA.

Unless otherwise specified, “autophagy receptor” mentioned in this application refers to proteins recognize and recruit specific cargoes to the autophagosome-lysosome pathway for degradation.

Protein aggregation is a hallmark of multiple human pathologies. Autophagy selectively degrades protein aggregates via aggrephagy. How selectivity is achieved has been elusive. Here the inventors identify the chaperonin subunit CCT2 as an autophagy receptor regulating the clearance of aggregation-prone proteins in the cell and the mouse brain. CCT2 associates with aggregation-prone proteins independent of cargo ubiquitination and interacts with autophagosome marker ATG8s through a non-classical VLIR motif. In addition, CCT2 regulates aggrephagy independent of the ubiquitin-binding receptors (P62, NBR1, and TAX1BP1) or chaperone-mediated autophagy. Unlike P62, NBR1, and TAX1BP1 which facilitate the clearance of protein condensates with liquidity, CCT2 specifically promotes the autophagic degradation of protein aggregates with little liquidity (solid aggregates). Furthermore, aggregation-prone protein accumulation induces the functional switch of CCT2 from a chaperone subunit to an autophagy receptor via promoting CCT2 monomer formation, which exposes the VLIR for ATG8s interaction and therefore, enables the autophagic function.

### EXAMPLES

KEY RESOURCES TABLE

| | | |
|---|---|---|
| REAGENT or RESOURCE | SOURCE | IDENTIFIER |

Antibodies

| | | |
|---|---|---|
| Mouse monoclonal anti-CCT1 | Boster | Cat# M02389 |
| Rabbit polyclonal anti-CCT2 | Abclonal | Cat# A6546, |
| | | RRID: AB_2767139 |
| Rabbit polyclonal anti-CCT3 | Boster | Cat# PB9926 |
| Rabbit polyclonal anti-CCT4 | Proteintech | Cat# 21524-1-AP, |
| | | RRID: AB_10733520 |
| Rabbit polyclonal anti-CCT5 | Proteintech | Cat# 11603-1-AP, |
| | | RRID: AB_2073774 |
| Rabbit polyclonal anti-CCT6A | Proteintech | Cat# 19793-1-AP, |
| | | RRID: AB_10638922 |
| Rabbit polyclonal anti-CCT7 | Proteintech | Cat# 15994-1-AP, |
| | | RRID: AB_2073903 |
| Mouse monoclonal anti-CCT8 | Proteintech | Cat# 67539-1-Ig, |
| | | RRID: AB_2882758 |
| Rabbit monoclonal anti-HA | CST | Cat# 3724, |
| | | RRID: AB_1549585 |
| Mouse monoclonal anti-HA | CST | Cat# 2367S, |
| | | RRID: AB_10691311 |
| Mouse monoclonal anti-T7 | Millipore | Cat# 69522, |
| | | RRID: AB 11211744 |
| Rabbit monoclonal anti-V5 | CST | Cat# 13202S, |
| | | RRID: AB_2687461 |
| Mouse monoclonal anti-LC3 | CST | Cat# M152-3, |
| | | RRID: AB 1279144 |
| Mouse monoclonal anti-SQSTM1/p62(WB) | Abcam | Cat# ab56416, |
| | | RRID: AB_945626 |
| Rabbit polyclonal anti-SQSTM1/p62(IF) | MBL | Cat# PM045, |
| | | RRID: AB_1279301 |
| Mouse polyclonal anti-NBR1 | Novus | Cat# H00004077-B01P, |
| | | RRID: AB_2149403 |
| Rabbit monoclonal anti-TAX1BP1 | CST | Cat# 5105, |
| | | RRID: AB_11178939 |
| Mouse monoclonal anti-Ubiquitin | CST | Cat# 3936, |
| | | RRID: AB_331292 |
| Rabbit monoclonal anti-GFP | CST | Cat# 2956, |
| | | RRID: AB_1196615 |
| Mouse monoclonal anti-GST | CST | Cat# 2624S, |
| | | RRID: AB_2189875 |
| Rabbit monoclonal anti-GFP | Abclonal | raised against purified GFP |
| Rabbit monoclonal anti-BFP | Abclonal | raised against purified BFP |
| Goat anti-GFP | Rockland | Cat# 600-101-215, |
| | | RRID: AB_218182 |
| Rabbit polyclonal anti-Ribophorin1(RPN1) | Dr. Randy | |
| | Schekman | |
| Rat monoclonal HSC70 | Abcam | Cat# Ab19136, |
| | | RRID: AB_444764 |
| Mouse monoclonal anti-ATG5 | MBL | Cat# M153-3, |
| | | RRID: AB_1278760 |
| Rabbit polyclonal anti-Beclin1 | Sigma | Cat# PRS3613, |
| | | RRID: AB_1845329 |
| Mouse monoclonal anti-alpha Tubulin | Abcam | Cat# ab7291, |
| | | RRID: AB_2241126 |
| Rabbit polyclonal anti-RB1CC1(FIP200) | Proteintech | Cat# 17250-1-AP, RRID: |
| | | AB_10666428 |
| Rabbit polyclonal to LAMP2A | Abcam | Cat# ab18528, |
| | | RRID: AB_775981 |

Chemicals, Peptides, and Recombinant Proteins

| | | |
|---|---|---|
| Bafilomycin A1 | Selleck | Cat# S1413 |
| Cycloheximide | CST | Cat# 2112S |
| Anti-T7 agarose | Millipore | Cat# 69026 |
| Anti-HA agarose | Sigma | Cat# A2095 |
| GFP-Trap magnetic beads | Chromotek | Cat# gtma-20 |
| Ni sepharose | GE Healthcare | Cat# 17-5318-02 |
| Glutathione beads | Smart | Cat# SA010100 |
| | Lifesciences | |
| Protein A/G PLUS-Agarose | SCBT | Cat# sc-2003 |
| LC3B protein (647) | Inventor's | N/A |
| | laboratory | |
| T7-LC3C protein | Inventor's | N/A |
| | laboratory | |
| T7-GABARAP protein | Inventor's | N/A |
| | laboratory | |
| T7-GABARAPLI protein | Inventor's | N/A |
| | laboratory | |
| CCT2 domain3 peptide 1: | Beijing SciLight | N/A |
| EACTIVLRGATQQILDEAERSLHDA | Biotechnology | |
| CCT2 domain3 peptide 2: | Beijing SciLight | N/A |
| ERSLHDALCVLAQTVKDSRTVYGGGCSE | Biotechnology | |
| CCT2 domain3 peptide 3: | Beijing SciLight | N/A |
| GGGCSEMLMAHAVTQLANRTPGKEA | Biotechnology | |
| CCT2 domain3 peptide 4: | Beijing SciLight | N/A |
| PGKEAVAMESYAKALRMLPTIIADN | Biotechnology | |
| CCT2 domain3 peptide 5: | Beijing SciLight | N/A |
| IIADNAGYDSADLVAQLRAAHSEGN | Biotechnology | |
| CCT2 domain3 peptide 6: | Beijing SciLight | N/A |
| HSEGNTTAGLDMREGTIGDMAILGI | Biotechnology | |
| CCT2 domain3 peptide 7: | Beijing SciLight | N/A |
| ILGITESFQVKRQVLLSAAEAAEVILRVDN | Biotechnology | |
| CCT2 domain3 peptide 7 mVL(I)L: | Beijing SciLight | N/A |
| ILGITESFQVKRQAAASAAEAAEAAARVDN | Biotechnology | |
| CCT2 domain3 peptide 8: | Beijing SciLight | N/A |
| AEVILRVDNIIKAAPRKRVPDHHPC | Biotechnology | |

Critical Commercial Assays

| | | |
|---|---|---|
| Duolink PLA kit | Sigma | Cat# DUO92102 |
| AminoLink Plus Coupling Resin | Thermo | Cat# 20501 |

Experimental Models: Cell Lines

| | | |
|---|---|---|
| HEK293T Cells | Dr. Randy | N/A |
| | Schekman | |
| U2OS Cells | Dr. Randy | N/A |
| | Schekman | |
| N2A Cells | Dr. Randy | N/A |
| | Schekman | |
| U2OS Q91-HTT-GFP Cells | Dr. Kirill Bersuker | |
| | (Dr. Ron Kopito | |
| | lab) | |
| N2A Q150-HTT-GFP Cells | Dr. Nukina | |
| | Nobuyuki | |
| MEF WT | Dr. Noboru | |
| | Mizushima | |
| MEF Atg5KO | Dr. Noboru | |
| | Mizushima | |

Oligonucleotides

| | | |
|---|---|---|
| CCT2 siRNA target sequence-1: | GenePharma | N/A |
| CCCACGTGCTGTCGATCTT | | |
| CCT2 siRNA target sequence-2: | GenePharma | N/A |
| GCTGACCTTCGCTTTAACA | | |
| CCT4 siRNA target sequence-1: | GenePharma | N/A |
| GGATTCATCCAACCATCAT | | |
| CCT4 siRNA target sequence-2: | GenePharma | N/A |
| GCACCATTATGATCACCAG | | |
| CCT4 siRNA target sequence-3: | GenePharma | N/A |
| GCCTGAAGTTGTATTGAAA | | |
| CCT5 siRNA target sequence-1: | GenePharma | N/A |
| GCATCGACTGTTTGCACAA | | |
| CCT5 siRNA target sequence-2: | GenePharma | N/A |
| CCATGTGAGCCTTTGCTTT | | |
| CCT5 siRNA target sequence-3: | GenePharma | N/A |
| GCTAATAGCAATCTTCCTA | | |
| Atg5 siRNA target sequence-1: | Qiagen | N/A |
| AACCTTTGGCCTAAGAAGAAA | | |
| Atg5 siRNA target sequence-2: | Qiagen | N/A |
| CTAGGAGATCTCCTCAAAGAA | | |
| Atg5 siRNA target sequence-3: | Qiagen | N/A |
| AAGACTTACCGGACCACTGAA | | |
| Atg5 siRNA target sequence-4: | Qiagen | N/A |
| CATCATAGCTTTATTACTCTA | | |
| Beclin1 siRNA target sequence-1: | Qiagen | N/A |
| GAGGATGACAGTGAACAGTTA | | |
| Beclin1 siRNA target sequence-2: | Qiagen | N/A |
| TGGACAGTTTGGCACAATCAA | | |
| Beclin1 siRNA target sequence-3: | Qiagen | N/A |
| AGGGTCTAAGACGTCCAACAA | | |
| Beclin1 siRNA target sequence-4: | Qiagen | N/A |
| ACCGACTTGTTCCTTACGGAA | | |
| P62 siRNA target sequence-1: | Qiagen | N/A |
| TGACGTTTGCATAGAGAGAAA | | |
| P62 siRNA target sequence-2: | Qiagen | N/A |
| TCGGAGGATCCGAGTGTGAAT | | |
| P62 siRNA target sequence-3: | Qiagen | N/A |
| CTCATAGGTCCCTGACATTTA | | |
| P62 siRNA target sequence-4: | Qiagen | N/A |
| TAGGGTGCAAGAAGCCATTTA | | |
| NBR1 siRNA target sequence-1: | RiboBio | N/A |
| GGAGTGGATTTACCAGTTA | | |
| NBR1 siRNA target sequence-2: | RiboBio | N/A |
| GGTGCAGTATCATAGTAGA | | |
| NBR1 siRNA target sequence-3: | RiboBio | N/A |
| GAGCCCTGATAACATTGAA | | |
| TAX1BP1 siRNA target sequence-1: | GenePharma | N/A |
| GCCTGAACATTATGTGGAA | | |
| TAX1BP1 siRNA target sequence-2: | GenePharma | N/A |
| GCTTACAACCTCAAGTAAA | | |
| TAX1BP1 siRNA target sequence-3: | GenePharma | N/A |
| GCAGCCAGCCTGCTCGAAA | | |
| HSC70 siRNA target sequence-1: | GenePharma | N/A |
| GTCCTCATCAAGCGTAATA | | |
| HSC70 siRNA target sequence-2: | GenePharma | N/A |
| GGCCAGTATTGAGATCGAT | | |

Recombinant DNA

| | | |
|---|---|---|
| pmCherry-Q91-HTT | Dr. Kirill Bersuker | |
| pEGFPN1-Q103-HTT | Inventor's | N/A |
| | laboratory | |
| pBFPN1-Q103-HTT | Inventor's | N/A |
| | laboratory | |
| pEGFPN1-Q103-HTT-APEX2 | Inventor's | N/A |
| | laboratory | |
| pFUGW-Q103-T7 | Inventor's | N/A |
| | laboratory | |
| pFUGW-Q103KR-T7 | Inventor's | N/A |
| | laboratory | |
| pRK5-EGFP-Tau P301L | Inventor's | N/A |
| | laboratory | |
| pEGFPN1-SOD1 G93A | Inventor's | N/A |
| | laboratory | |
| pEGFPC1-FUS WT | Inventor's | N/A |
| | laboratory | |
| pEGFPC1-FUS P525L | Inventor's | N/A |
| | laboratory | |
| pEGFPC1-FUS P525L + 16R | Inventor's | N/A |
| | laboratory | |
| pCDH-mCherry-LC3B | Inventor's | N/A |
| | laboratory | |
| pCDH-mCherry-LC3B G120A | Inventor's | N/A |
| | laboratory | |
| pFUGW-mCherry-pHluorin-LC3B | Inventor's | N/A |
| | laboratory | |
| pCDNA3-T7-LC3A | Inventor's | N/A |
| | laboratory | |
| pCDNA3-T7-LC3B | Inventor's | N/A |
| | laboratory | |
| pCDNA3-T7-LC3C | Inventor's | N/A |
| | laboratory | |
| pCDNA3-T7-GABARAP | Inventor's | N/A |
| | laboratory | |
| pCDNA3-T7-GABARAPL1 | Inventor's | N/A |
| | laboratory | |
| pCDNA3-T7-GABARAPL2 | Inventor's | N/A |
| | laboratory | |
| pFUGW-HA-CCT1 | Inventor's | N/A |
| | laboratory | |
| pFUGW-HA-CCT2 | Inventor's | N/A |
| | laboratory | |
| pFUGW-HA-CCT2 Δpep7 | Inventor's | N/A |
| | laboratory | |
| pFUGW-HA-CCT2 mVLL | Inventor's | N/A |
| | laboratory | |
| pFUGW-HA-CCT2 mVIL | Inventor's | N/A |
| | laboratory | |
| pFUGW-HA-CCT2 mVL(I)L | Inventor's | N/A |
| | laboratory | |
| pFUGW-HA-CCT2 T400P | Inventor's | N/A |
| | laboratory | |
| pFUGW-HA-CCT2 R516H | Inventor's | N/A |
| | laboratory | |
| pFUGW-V5-CCT2 | Inventor's | N/A |
| | laboratory | |
| pFUGW-V5-CCT2 T400P | Inventor's | N/A |
| | laboratory | |
| pCDH-GFP-CCT2 | Inventor's | N/A |
| | laboratory | |
| pCDH-GFP-CCT2 D1 | Inventor's | N/A |
| | laboratory | |
| pCDH-GFP-CCT2 D2 | Inventor's | N/A |
| | laboratory | |
| pCDH-GFP-CCT2 D3 | Inventor's | N/A |
| | laboratory | |
| pFUGW-HA-CCT3 | Inventor's | N/A |
| | laboratory | |
| pFUGW-HA-CCT4 | Inventor's | N/A |
| | laboratory | |
| pFUGW-HA-CCT5 | Inventor's | N/A |
| | laboratory | |
| pFUGW-HA-CCT6 | Inventor's | N/A |
| | laboratory | |
| pFUGW-HA-CCT7 | Inventor's | N/A |
| | laboratory | |
| pFUGW-HA-CCT8 | Inventor's | N/A |
| | laboratory | |
| pCMV3-HSPA9-HA | SinoBiological | HG16926-CY |
| pCMV3-HSPD1-HA | SinoBiological | HG11322-CY |
| pCMV3-HA-HSP90AA1 | SinoBiological | HG11445-NY |
| pCDNA3-HSP90AB1-HA | addgene | 22487 |
| pcDNA3-HSP90B1-HA | Inventor's | N/A |
| | laboratory | |
| pCMV3-HA-HSPA4L | SinoBiological | HG20756-NY |
| pCMV3-HSPH1-HA | SinoBiological | HG12215-CY |
| pCMV3-DNAJA3-HA | SinoBiological | MG51440-CY |
| pCMV3-DNAJB2-Flag | SinoBiological | HG20425-CF |
| pCMV3-PPIA-HA | SinoBiological | HG10436-CY |
| pCDNA3-VCP-HA | Inventor's | N/A |
| | laboratory | |
| pCMV3-STIP1-HA | SinoBiological | HG16371-CY |
| pCDNA3-ANAPC7-HA | Inventor's | N/A |
| | laboratory | |
| pFUGW-HA-D2-V5-D3 | Inventor's | N/A |
| | laboratory | |
| pFUGW-HA-D2-P7 | Inventor's | N/A |
| | laboratory | |

Software and Algorithms

| | | |
|---|---|---|
| Fiji (ImageJ) | | https://imagej.nih.gov/ij/ |
| Prism 8 | GraphPad | https://www.graphpad.com |
| Flowjo | FLOWJO | https://www.flowjo.com |
| Imaris 9 | IMARIS | https://imaris.oxinst.com |
| Zeiss Zen Blue 3.1 | ZEISS | https://www.zeiss.com.cn |

### Method

Cells HEK293T, U2OS, and N2A cells were maintained in DMEM supplemented with 10% FBS at 37° C. in 5% CO₂. For induction of Q91-HTT-mCherry expression, U2OS HTT-Q91-mCherry cells were incubated with 1 μg/ml doxycycline for 24 h. For induction of Q150-HTT-GFP expression, N2A Q150-HTT-GFP cells were differentiated with 5 mM dbcAMP for 24 h followed by 1 μM ponasterone A for 48 h. The cells were employed for in vitro reconstitution, immunofluorescence, electron microscopy, and biochemical assays as described below. Transfection of DNA constructs was performed using PEI (Polysciences, Inc.) for HEK293T and X-tremeGENE HP (Roche) for U2OS and N2A. The siRNA transfection was performed with Lipofectamine RNAiMAX (Invitrogen) as described previously.

For primary culture of mouse striatal neurons, mouse striatal neurons were dissected from newborn WT mice and incubated in 0.25% trypsin-ethylenediaminetetraacetic acid (Life Technologies) for 15 min at 37° C. After washing with Hank's Buffered Salt Solution plus 5 mM Hepes (Life Technologies), 20 mM D-glucose, and 2% fetal bovine serum (FBS) (Gibco), the neurons were mechanically dissociated in culture medium and plated on poly-D-lysine-coated glass coverslips at a density of 50,000 to 100,000 cells/cm². Cells were grown in Neurobasal-A medium (Life Technologies) supplemented with 2% B-27 (Life Technologies) and 2 mM glutamax (Life Technologies). Cultures were maintained at 37° C. in a 5% CO₂-humidified incubator. AAV viruses were added to neurons at day in vitro (DIV) 3, and the chase assay was performed as described below at DIV8.

### Mice

The Hdh140Q knock-in mice was a gift from Boxun Lu. The generation and characterization of the Hdh140Q knock-in mice have been previously described. The mice were housed in ventilated cages in a temperature and light regulated room in a SPF facility and received food and water ad libitum. The mouse experiments were approved by the Institutional Animal Care and Use Committees at Tsinghua University and they were in compliance with all relevant ethical regulations.

### In Vitro Reconstitution

The in vitro reconstitution contains steps of protein purification, fluorescence labeling, and in vitro LC3 recruitment assay. Protein purification was described before. In brief, His-tagged LC3 protein with a cysteine interaction in the N-terminus for fluorophore maleimide labeling was expressed in E. coli. BL21 and purified using Nickel Sepharose (GE). The LC3 protein was labeled with Alexa Fluor 647/488 C2 maleimide (Invitrogen) according to the manual provided and subsequently gel filtrated to remove the unlabeled fluorophore. For in vitro reconstitution of LC3 recruitment to the IBs in the cell, U2OS HTT-Q91-mCherry or N2A HTT-Q150-GFP cells were plated on a coverslip (for immunofluorescence), and fluorescence-tagged PolyQ-HTT IBs were induced for 24-48 h. The cells were then treated with 40 μg/ml digitonin on ice to permeabilize the plasma membrane, incubated with 5-10 μg/mL fluorescence-labeled LC3 for 1 h at 30° C., and fixed by 4% paraformaldehyde (PFA) for microscopy analysis. For in vitro reconstitution of LC3 to IB in solution, the cells with IBs were harvested and lysed in B88 (20 mM HEPES (pH 7.2), 250 mM sorbitol, 150 mM potassium acetate, 5 mM magnesium acetate) with 1% Triton X-100, protease inhibitors, DNase and RNase. The lysate was centrifuged at 300×g. The pellet containing the IBs was collected and incubated with 5-10 μg/mL fluorescence-labeled LC3 for 1 h at 30° C. after which FACS was performed to analyze LC3 recruitment to IBs.

### FACS Analysis, Sorting of IBs and Mass Spectrometry-Based Label-Free Quantification

To analyze LC3 recruitment to IBs, U2OS HTT-Q91-mCherry or N2A HTT-Q150-GFP cells were plated in 10 cm dishes and fluorescence-tagged PolyQ-HTT IB was induced for 24-48 has described above. The cells were harvested by centrifugation and lysed in B88 with 1% Triton X-100, protease inhibitors, DNase, and RNase by passaging through a 22G needle for 10 times. The lysate was then centrifuged at 300×g for 10 min. The pellet containing the IBs was collected and incubated with 0.5-1 μg/mL fluorescence-labeled LC3 in B88 with protease inhibitors for 1 h at 30° C. The reaction mixture was centrifuged at 1000×g for 5 min and suspended in B88 with 1% Triton X-100 to wash the pellet, followed by centrifugation at 1000×g for 5 min. Finally, the pellet was suspended in B88 with 1% Triton X-100 and FACS analysis (PulSA, BD Fortessa) or sorting (BD Influx) was performed as described previously with modifications described in figure legends. After sorting, the IB solutions were centrifuged at 3000×g for 30 min, and pellet were analyzed by immunoblot or mass spectrometry in Taplin Biological Mass Spectrometry Facility at Harvard Medical School.

To quantify the known receptors and CCT2 on IBs or in cells, N2A HTT-Q150-GFP cells were plated in 10 cm dishes and fluorescence-tagged PolyQ-HTT IB was induced for 48 h. The cells were harvested by centrifugation and lysed in HB1 buffer (20 mM HEPES-KOH, pH 7.2, 400 mM Sucrose, 1 mM EDTA) with 1% Triton X-100, protease inhibitors, DNase, and RNase by passaging through a 22G needle for 10 times. The lysate was then centrifuged at 300×g for 10 min. The pellet containing the IBs was suspended with PBS. IBs or IB-positive cells were sorted by BD FACSAria SORP. After sorting, the IB and cell solutions were centrifuged at 3000×g for 30 min.

Mass spectrometry analysis was performed at the Protein Chemistry and Proteomics Center at Tsinghua University. In brief, the IB proteins (IB group) and total cell proteins (cell group) were resolved in SDS-PAGE and stained by Simply Blue (Invitrogen). The lanes were excised from the gel, reduced, alkylated, and digested with trypsin overnight. The resulting tryptic peptides were analyzed using an UltiMate 3000 RSLCnano System (Thermo Scientific, USA) which was directly interfaced with a Thermo Orbitrap Fusion Lumos mass spectrometer (Thermo Fisher Scientific, USA). The RAW files were searched against the Mouse Proteome (Uniprot) database using an in-house Proteome Discoverer 2.3 searching algorithm. The peak area was used for protein abundance comparison between the IB group and the cell group. The iBAQ value calculated by Maxquant was used to estimate the protein content in IB group.

Plasmids and siRNA Oligos

Q91-HTT-mcherry plasmid was a gift from Dr. Kirill Bersuke. We obtained Q103-HTT from Dr. Bing Zhou and the Q103-HTT-GFP plasmid was generated by PCR and ligation. SOD1-encoding DNA was amplified from HEK293T cDNA and the SOD1 (G93A)-GFP plasmid was constructed by site mutagenesis PCR. The Tau plasmid was obtained from Addgene (46904). Tau-GFP (P301L) mutant was generated by site mutagenesis PCR. FUS and FUS (P525L) were from Dr. Cong Liu. FUS 16R was described previously. The pEGFPC1-FUSs plasmids were generated by PCR, ligation and site mutagenesis PCR. The CCT1-8 encoding genes were PCR amplified rom HEK293T cDNA and inserted into the FUGW vector with different tags at the N-terminus. Mutagenesis was formed by PCR. ATG8 family protein genes were amplified by PCR and inserted into the plasmids for mammalian expression. HSPA9, HSPD1, HSP90AA1, HSPA4L, HSPH1, DNAJA3, DNAJB2, PPIA, and STIP1 plasmids were purchased from Sinobiological, and HSP90AB1 plasmid from Addgene. The VCP and ANAPC7 were PCR amplified from templates (VCP from Dr. Bao-Liang Song, ANAPC7 from Sinobiological). The HSP90B1 was described as previously.

For siRNAs, the targeting sequences for human CCT2, CCT4, CCT5, ATG5, Beclin1, P62, NBR1, TAXIBP1, and HSC70 were shown above. An equimolar mixture of different siRNAs for a specific gene was used to induce gene silencing. AllStars negative siRNA (GenePharma) was used as a control.

### CHX Chase Assay

Cells were transfected with indicated plasmids. After transfection for the indicated times (in Figure legends), cells were treated with 50 μg/mL CHX, with or without 0.5 μg/mL Bafilomycin Al as indicated and were collected at each indicated time point for immunoblot analysis. For the insoluble Q103-HTT detection, cells were permeabilized with 40 μg/mL of digitonin diluted in PBS on ice for 5 min and washed with PBS before being collected for immunoblot analysis.

### Q140 Mice and AAV Injection

For determination of Q140-HTT via immunoblot, AAVs (CCT2 and mCherry) were delivered to the striatum. Briefly, Hdh140Q mice were anesthetized by an i.p. injection with avertin and immobilized on rodent stereotaxic frames. A burr hole was used to perforate the skull, and the AAVs (400nl per injection spot, 5×1012vg/ml) were injected into the striatum using a 10 μl syringe at a rate of 50 nL/min. The injection coordinates were Anterior/Posterior (AP)+0.9 mm, Medial/Lateral (ML)+/−1.8 mm from the bregma, and Dorsal/Ventral (DV)-2.7 mm from the dura. Striatal tissues of Hdh140Q mice were carefully removed for immunoblot analyses at 2 months post AAV injection. For determination of HTT-IBs, Hdh140Q mice (mixed gender) received bilateral intrastriatal injections of AAV constructs encoding GFP, HA-CCT2 WT, or HA-CCT2 R516H at 2 months of age. Mice were individually anaesthetized with Avertin and placed in a stereotaxic instrument. A longitudinal mid-sagittal incision of length 1 cm was made in the scalp, after sterilization with 75% ethanol and iodine solution. Following skin incision, a small hole corresponding to the striatal injection site was made in the skull using an electrical drill. The coordinates measured according to the mouse bregma were 0.8 mm anterior, 1.8 mm lateral and 3.8 mm deep with flat skull nosebar setting. A total volume of 300 nL (1×10⁹ genome copies) viral vectors were administered using a Hamilton gas-tight syringe connected to an automated micro-injection pump at a constant flow rate of 50 nL/min. After injection, the surgical wound was sealed and the animal was kept on a heating pad until fully recovered. For experiments using R6/2 transgenic mice, at 3 weeks of age, AAV-CAG-GFP, AAV-CAG-HA-CCT2 WT or AAV-CAG-HA-CCT2 R516H was bilaterally delivered to the striatum of R6/2 mice using stereotaxic injection.

### Histology and Immunohistochemistry

Mice were euthanized at 4 months by transcardial perfusion. For perfusions, mice were deeply anesthetized by intraperitoneal injection of Avertin using a 27-gauge needle. Before perfusion, animals were assessed for loss of toe pinch reflex to ensure that the correct level of anesthesia was achieved. Mice were transcardially perfused with 20 mL of ice-cold PBS followed by 30 mL of 4% paraformaldehyde using a peristaltic pump. Brain samples were removed from the skull and post-fixed overnight in the same fixztive at 4° C., and cryoprotected by incubation in 30% sucrose solution until saturated. Whole brains were embedded in TissueTek and stored at −80° C. Coronal sections of 20 μm were cut using a cryostat, collected as free-floating in 24-well plates and directly used for staining or stored in a cryoprotection solution (50% PBS, 30% ethylene glycol, 20% glycerol) at −20° C. until time of use. The following primary antibodies were used for immunostaining: monoclonal mouse anti-mutant huntingtin, monoclonal rabbit anti-HA. Sections were permeabilized in 0.1% Triton X-100/PBS, blocked in 3% BSA/PBS and incubated with the primary antibody diluted in the blocking buffer at 4° C. overnight. Sections were washed three times in 0.1% Triton X-100/PBS for 30 min and incubated in the secondary antibody for 2 h at room temperature. Sections were washed in 0.1% Triton X-100/PBS as described above and mounted using aqueous mounting medium containing DAPI.

### Open Field Test

R6/2 transgenic mice were subjected to open field testing at 6, 8, 10 and 12 weeks of age. Animals were placed in square, acrylic chambers for 30 min. Total horizontal activity (distance traveled) were measured.

### Protein Purification

The His-T7-LC3C/GABARAP/GABARAPL1, His-CFP/Q45-CFP, His-mRuby2/mRuby2-CCT2, and MBP-TEV-GFP-FUS P525L proteins were purified using Ni sepharose (GE Healthcare), and the GST, GST-HA-CCT2s and GST-P62 proteins were purified using Glutathione beads as described before. The Ub8 protein was gift from Dr. Li Yu.

### Co-Immunoprecipitation, In Vitro Peptide/Protein Pull-Down Assay and Immunoblot

Co-immunoprecipitation was performed essentially as described before. In brief, 24 h after transfection, the cells were collected and lysed on ice for 30 min in co-IP buffer (50 mM Tris/HCl pH 7.4, 150 mM NaCl, 1 mM EDTA, 0.5% NP40) with protease inhibitor mixture, and the lysates were cleared by centrifugation. The resulting supernatants were incubated with indicated agarose or magnetic beads and rotated at 4° C. for 3 h. The agarose was washed five times with co-IP buffer. As for the BFP-tagged Q103, the supernatants were incubated with rabbit anti-BFP antibodies and Protein A/G PLUS-Agarose according to the manufacturers' protocol. After washing, 2×SDS loading buffer was added to the beads, and immunoblot was performed as described previously.

For peptide pull-down assay, synthetic peptides were conjugated to agarose beads using the AminoLink Plus Coupling Resin (Thermo, Cat #20501) according to the manufacturers′rotocol. 2 μg purified T7-tagged LC3C proteins were incubated with 15 μL peptides-coupled beads in co-IP buffer and rotated at 4° C. for 3 h. Then the agarose was washed three times with co-IP buffer. After washing, 2×SDS loading buffer was added to the beads, and immunoblot was performed as described previously.

For in vitro protein pull-down assay, 20 μg purified His-T7-LC3C protein was incubated with 20 μL Ni sepharose in PBS for 1 h on a rotor at 4° C. After washing, the beads were incubated with 5 μg GST-CCT2s proteins or the fractions after gel-filtration for 3 h on a rotor at 4° C. After washing, 2×SDS loading buffer was added to the beads, and immunoblot was performed. As for the GST-pull down of polyubiquitin chains, 200 pmol purified GST or GST tagged proteins were incubated with Glutathione beads in co-IP buffer for 2 h on a rotor at 4° C. After washing, the beads were incubated with 5 pmol Ub8 protein or the cell lysate from MG132 treated HEK293T cells for 3 h on a rotor at 4° C. After washing, beads were eluted with elution buffer (50 mM Tris/HCl PH 8.0, 20 mM GSH). 5×SDS loading buffer was added to the elutions, and immunoblot was performed.

### Immunofluorescence and Duolink PLA

Immunofluorescence was performed as previously described. In brief, the cells were permeabilized with 40 μg/mL of digitonin diluted in PBS on ice for 5 min, washed once with cold PBS and immediately incubated with 4% PFA for 20 min at room temperature. The cells were further permeabilized with 50 μg/mL of digitonin diluted in PBS at room temperature for 10 min followed by blocking with 10% FBS diluted with PBS for 1 h and primary antibody incubation for 1 h. The cell was washed three times with PBS, followed by secondary antibody incubation for 1 h at room temperature. Fluorescence images were acquired using the Olympus FV3000 confocal microscope. Quantification was performed using ImageJ software.

Duolink PLA was performed as described previously. In brief, 24 h after transfection, the cells were fixed with 4% paraformaldehyde for 20 min and permeabilized with 0.1% Triton X-100 diluted in PBS at room temperature. The cell was blocked with 10% FBS, incubated with primary antibodies and PLA probes followed by ligation and amplification using the recommended conditions according to the manual. Images were captured by Olympus FV3000 confocal microscope, and the quantification was performed using ImageJ software.

### Electron Microscopy (EM), Correlative Light and Electron Microscopy (CLEM), and DAB Staining

U2OS cells were transfected with Q103-HTT-GFP and either empty plasmids or HA-CCT2.24-48h after transfection, cells were fixed with 2.5% glutaraldehyde for 1 h at room temperature and washed 3×15 min with 0.1M PB (0.02M NaH₂PO₄, 0.08M Na₂HPO₄, PH 7.4). Post-fixation staining was performed with 1% osmium tetroxide (SPI, 1250423) for 0.5 h on ice. Cells were washed 3×15 min with ultrapure water, and then placed in 1% aqueous uranyl acetate (EMS, 22400) at 4° C. overnight. Samples were then washed 3×15 min with ultrapure water, and dehydrated in a cold-graded ethanol series (50%, 70%, 80%, 90%, 100%, 100%, 100%; 2 min in each). Penetrating in EPON 812 resin using 1:1 (v/v) resin and ethanol for 8 h, 2:1 (v/v) resin and ethanol for 8 h, 3:1 (v/v) resin and ethanol for 8 h, then pure resin 2× 8 h and finally into fresh resin and polymerisation in oven at 60° C. for 48 h. Embedded samples were sliced into 80-nm-thick sections and stained with uranyl acetate and lead citrate (C1813156). Samples were imaged under the H-7650 80kv transmission electron microscope.

For CLEM, U2OS cells were seeded in a gridded glass bottom dish (Cellvis, D35-14-1.5GI), and co-transfected with Q103-HTT-BFP, GFP-CCT2, and mcherry-LC3.24 h after transfection, cells were fixed with 4% PFA for 20 min at room temperature. Fluorescence images were captured by Olympus FV3000 confocal microscope. The cell shape and the position of ROI were acquired and recorded under bright field. After imaging, the cells were fixed with 2.5% glutaraldehyde for 1 h at room temperature. Samples for TEM were prepared as described above. The grids were engraved on the resin surface allowing for the location of ROIs on the resin surface. The samples of ROI were cut into 80-nm-thick sections. Stained sections were observed with the H-7650 80kv transmission electron microscope. Finally, the fluorescence images and TEM images were overlaid using Zeiss Zen Blue software.

For DAB staining, cells were fixed with room temperature 2.5% glutaraldehyde in buffer (100 mM sodium cacodylate with 2 mM CaCl₂), pH7.4) and quickly moved to ice. Cells were kept between 0 and 4° C. for all subsequent steps until resin infiltration. After 30 min, cells were rinsed 5×2 min in chilled buffer, and then treated for 5 min in buffer containing 20 mM glycine to quench unreacted glutaraldehyde followed by 5×2 min rinses in chilled buffer. A freshly diluted solution of 0.5 mg/mlL (1.4 mM) DAB tetrahydrochloride ((Sigma, 32750) was combined with 0.03% (v/v) (10 mM) H₂O₂ in chilled buffer, and the solution was added to cells for 5 min. To halt the reaction, the DAB solution was removed, and cells were rinsed 5×5 min with chilled buffer. Samples for TEM were prepared as described above. DAB-stained areas of embedded cultured cells were identified by transmitted light and cut into 80-nm-thick sections. The samples were observed with the H-7650 80kv transmission electron microscope.

### Membrane Fractionation

The procedure is modified from our previous work. HEK293T cells were transfected with indicated plasmids and harvested after 24 hours. Cells were then homogenized in a 2x cell pellet volume of HB1 buffer plus a cocktail of protease and phosphatase inhibitors (Roche, Indianapolis, IN) and 0.3 mM DTT by passing through a 22 G needle until ˜85% lysis analyzed by Trypan Blue staining. Homogenates were subjected to sequential differential centrifugation at 3,000×g (10 min) and 25,000 xg (20 min) to achieve the 25,000 xg membrane pellet (25K). The 25K pellet was suspended in 0.25 mL 1.25 M sucrose buffer and overlaid with 0.25 mL 1.1 M and 0.2 mL 0.25 M sucrose buffer (Golgi isolation kit; Sigma). Centrifugation was performed at 120,000×g for 2 h (TLS 55 rotor, Beckman), after which two fractions, one at the interface between 0.25 M and 1.1 M sucrose (L fraction) and the pellet on the bottom (P fraction), were separated. The L fraction which contained the highest level of LC3-II was suspended in 0.2 mL 19% OptiPrep for a step gradient containing 0.1 mL 22.5%, 0.2 ml 19% (sample), 0.18 mL 16%, 0.18 mL 12%, 0.2 mL 8%, 0.1 mL 5% and 0.04 mL 0% OptiPrep each each. Each density of OptiPrep was prepared by diluting 60% OptiPrep (20 mM Tricine-KOH, pH 7.4, 42 mM sucrose and 1 mM EDTA) with a buffer containing 20 mM Tricine-KOH, pH 7.4, 250 mM sucrose and 1 mM EDTA. The OptiPrep gradient was centrifuged at 150,000×g for 3 h (TLS 55 rotor, Beckman) and subsequently ten fractions, 0.1 mL each, were collected from the top. 5×SDS loading buffer was added to the fractions, and immunoblot was performed with the indicated antibodies.

### Proteinase K Protection Assay

The autophagosome fractions from membrane fractionation were collected and suspended in B88 buffer and divided into three fractions (without proteinase K, with proteinase K (80 μg/mL), and with proteinase K and 0.5% Triton X-100) 20 μL per fraction. The reactions were performed on ice for 20 min and stopped by adding PMSF and 2×SDS loading buffer. The samples were immediately heated at 100° C. for 10 min, and immunoblot was performed with the indicated antibodies.

### Filter Trap Assay

The Filter Trap assay was performed refered to a described protocol. In Brief, cells were collected and lysed in FTA lysis buffer (10 mM Tris-HCl, PH 8.0, 150 mM NaCl, 2% SDS, 50 mM DTT) and heated at 100° C. for 10 min. The filter papers and 0.2 μm pore size cellulose acetate membrane (Sterlitech) were soaked in FTA wash buffer (10 mM Tris-HCl, PH 8.0, 150 mM NaCl, 0.1% SDS), and placed on the base of the MINIFOLD I 96 well Dot-Blot System (GE Healthcare), with the cellulose acetate membrane on top of the filter papers. After washing wells with FTA wash buffer, samples were loaded and washed with FTA wash buffer, each step above were applied vacuum until the wells were empty. Following immunodetection of protein aggregates on cellulose acetate membrane was the same as immunoblot.

Fluorescence Recovery after Photobleaching (FRAP)

FRAP experiments were performed on Olympus FV3000 confocal microscope. FUS condensates were bleached for 5 s using a laser intensity of 80% at 480 nm. Recovery was recorded for the indicated time durations. The fluorescence intensity of the photobleached area was normalized to the intensity of the unbleached area.

### In Vitro FUS Phase Separation, Aggregation and CCT2 Recruitment

For phase separation, 2 μM MBP-TEV-GFP-FUS P525L proteins were digested with TEV in phase separation buffer (40 mM Tris/HCl PH7.4, 150 mM KCl, 2.5% glycerol) for 1 hour. For aggregation, the proteins were shaked at 700 rpm in a shaker at 25° C. after TEV digestion. The products were transferred into 384-well glass bottom plate, 4 μM mRuby2 or mRuby2-CCT2 proteins were added and incubated for 5 min before imaging.

### Gel-Filtration

The cells were collected and lysed on ice for 30 min in co-IP buffer with protease inhibitor mixture, and the lysates were cleared by centrifugation. The supernatants were injected into a Superose 6 Increase 10/300 GL (GE Healthcare) exclusion column in an AKTA FPLC system (GE Healthcare). Samples were separated at a flow rate of 0.5 mL/min by co-IP buffer. Fractions were collected per 1 mL followed by analysis with immunoblot.

### Quantification and Statistical Analysis

Quantification of each experiment has been provided in the METHOD DETAILS. The statistical information of each experiment, including the statistical methods, the P-values and numbers were shown in the figures and corresponding legends. Statistical analyses were performed in GraphPad Prism.

results

### Identification of CCT2, a Chaperonin Subunit Responsible for LC3 Targeting to Inclusion Bodies

Selective targeting of the autophagic membrane to protein aggregates is an essential step in aggrephagy. To dissect this process, the inventors developed an in vitro reconstitution system to recapitulate autophagic membrane targeting to protein aggregates (FIG. 1A). It has been shown that autophagy receptors recruit autophagic membranes via associating with the ATG8 family members on the autophagosome. In this system, the inventors determined the recruitment of a fluorescence-labeled ATG8 family member LC3 to the PolyQ-huntingtin (HTT) inclusion bodies (IBs) which contain multiple properties of protein aggregates and have been extensively investigated for aggrephagy (FIG. 1A). Similar to the autophagy receptor P62, the fluorescent LC3 was attached to the IBs (FIG. 1B). In addition, IB association of the fluorescent LC3 was competed by unlabeled LC3 instead of BSA or FBS, indicating binding site sp ecificity of LC3 on the IB (FIGS. 1C and 1D). To quantitatively analyze the recruitment of LC3 to the IB, the inventors employed a pulse shape analysis (PulSA) based on flow cytometry. Consistently, the fluorescent LC3 was recruited to the IB, which was specifically inhibited by the unlabeled LC3. Therefore, certain components on the IB specifically associate with LC3.

Interestingly, the inventors observed different LC3 recruitment among IBs, indicating variable amounts of LC3-attracting components among individual IBs (FIG. 1C, compare arrow with arrowhead-pointed IBs). The observation inspired us to incorporate and develop a fluorescence-activated particle sorting (FAPS) system based on flow cytometry to isolate IBs with high (H) and low (L) LC3 association from two different cell lines, a differentiated mouse neuroblastoma cell line N2A with the Q150-HTT-GFP and a human osteosarcoma cell line U2OS with Q91-HTT-mCherry (FIGS. 1E-1G). In the immunoblot assay, the H-LC3 IBs contained a higher amount of LC3 as well as P62 and NBR1, confirming the feasibility of the FAPS system (FIG. 1H). Subsequently, the inventors employed an unlabeled quantitative mass spectrometry approach to compare protein components enriched in H- and L-LC3 IBs (FIGS. 1I and 1J). Consistent with the above immunoblot assay, P62 and NBR1 were enriched in the H-LC3 IBs (FIG. 1J). In addition, TAXIBP1, a recently identified new ubiquitin-binding aggrephagy receptor also appeared in the H-LC3 IBs (FIG. 1J). Another two reported ubiquitin-binding aggrephagy receptors, Optineurin and Tollip, were detected without enrichment to the H-LC3 IBs likely because our in vitro assay could not recapitulate the function of the two receptors.

Interestingly, the inventors found multiple chaperones and co-chaperones enriched in the H-LC3 IBs. These chaperones and co-chaperones were highly overlapped between the H-LC3 IBs of N2A and U2OS (FIG. 1K, 11 overlap of 19 in N2A and 13 in U2OS respectively). The inventors determined the effects of the chaperones or co-chaperones on autophagosome association with polyQ-HTT IBs and found that 9 out of the 21 analyzed chaperones or co-chaperones significantly increased the association of LC3 puncta (an indicator of autophagic membrane) with the IBs (FIG. 1L). Of the 9 chaperones, 6 dramatically promoted lysosome-dependent polyQ-HTT degradation in a chase assay with cycloheximide (CHX) inhibition of protein synthesis employed before to determine autophagy turnover (FIG. 1L). Of the 6 chaperones, 4 are chaperonin subunits (CCT1, CCT2, CCT3, and CCT6), and the other 2 contain HSP90AB1, a cytosolic chaperone, and HSPA9, a multi-location chaperone primarily in the mitochondria (FIG. 1L, arrowhead pointed).

The inventors next focused on CCT2 because: 1) CCT2 was the most enriched chaperone in the mass spectrometry and had the strongest effect on promoting autophagosome association with the IB and lysosome-dependent HTT clearance (FIGS. 1J and 1L); 2) In the PulSA assay mentioned above, knockdown (KD) of CCT2 decreased LC3 association with IBs and vice versa with expression of exogenous CCT2, suggesting a major contribution of CCT2 to LC3 recruitment to IBs in the in vitro assay (FIGS. 1M and IN); 3) In our preliminary data, IBs from glucose starvation-treated cells showed increased LC3 recruitment and mass spectrometry analysis also found the enrichment of CCT2 in the IBs from glucose starvation-treated cells (data not shown); 4) In a label-free mass spectrometry quantification, CCT2 (6-fold lower than P62 but 10-folded and 25-fold higher than NBR1 and TAX1BP1) appeared to be the highest compared to the other four chaperones (CCT1,CCT2, CCT6, and HSPA9) that associates with ATG8s (FIG. 10). Although the amount of HSP90AB1 is higher than CCT2, it did not associate with ATG8s and was not further studied (FIG. 5D).

### CCT2 Targets Autophagic Membrane to Aggregates and Promotes Aggrephagy

Around 10% of endogenous CCT2 (versus ˜70% of P62) localizes on the IBs in N2A cells (FIG. 1P). Similar to P62 (FIG. 1B), the exogenously expressed CCT2 associated with IBs (FIG. 2A). In addition, expression of CCT2 increased LC3 puncta with IBs (˜2.5-fold increase, FIGS. 2A and 2B). Similarly, the endogenous CCT2 also associated with IBs, and the amount of CCT2 around the IBs correlated with the amount of LC3 puncta association (FIGS. 3A and 3B). The IB-associated LC3 puncta requires LC3 lipidation, as lipidation-deficient LC3 mutant (G120A) failed to form puncta associated with IBs in the presence and absence of digitonin permeabilization to remove cytosolic components (FIGS. 2C and 2D). The inventors also observed colocalization of both WT and G120A mutant LC3 (diffused signal but not clear puncta) with the IB when co-expressed with Q103-HTT (FIG. 2C), which reflects the previous results showing that the unlipidated LC3 co-aggregates with protein aggregates. Consistent with the requirement of LC3 lipidation, the CCT2-promoted LC3 puncta around the IB was not observed in Atg5 knockout cells (FIGS. 2E and 2F).

In electron microscopy (EM), expression of CCT2 increased recruitment of autophagic vacuoles to the IBs compared to the control (˜2 fold increase, FIGS. 3C and 3D). The presence of autophagic membrane-like vacuoles on IBs was also confirmed by correlative light electron microscopy (CLEM) (FIG. 3E). The data indicate that CCT2 promotes autophagic membrane targeting to protein aggregates. The IB-associated LC3 puncta increased by CCT2 colocalized with FIP200/RB1CC1 and LAMP2, confirming that these puncta are autophagosomes and these autophagosomes could fuse with the lysosome (FIGS. 3F and 2G). In addition, CCT2 expression increased lysosome (labeled by LAMP2) association with the IB (FIGS. 3G and 3H).

To test if CCT2 promotes autophagic engulfment of Q103-HTT, The inventors performed Apex2 labeling of Q103-HTT. In the EM analysis, The inventors observed more Apex2-positive signals in autophagic vacuoles in cells with CCT2 expression compared to the control (FIGS. 2H and 21, ˜2.5 fold of increase). The autophagosome encapsulation was abolished in Atg5 KD cells or cells treated with SAR405 (FIGS. 2H and 21), a class III phosphatidylinositol-3 kinase inhibitor that blocks autophagy in the early stage. In a membrane fraction approach, CCT2 increased the amount of Q103-HTT in the autophagosome fraction (FIGS. 2J-2L, 2.5-fold and 3.9-fold before and after proteinase K digestion). Both Q103-HTT and CCT2 were protected from proteinase K digestion indicating that they are inside the autophagosome (FIG. 2L). These data together demonstrate that CCT2 promotes Q103-HTT entry into the autophagosome.

In the chase analysis as described above. Expression of CCT2 enhanced Q103-HTT degradation, which was blocked by the lysosome inhibitor Bafilomycin Al in U2OS, N2A, and primary cultured striatal neuron (FIGS. 31-3N). In a Huntington Disease mice with polyQ (Q140) knockin, expression of CCT2 reduced the level of endogenous polyQ-HTT (HTT-Q140) in the striatum (FIG. 2M). Knockdown of Atg5 and Beclin-1, two major autophagy regulators, abolished the effect of CCT2 on Q103-HTT degradation confirming the notion that CCT2 regulates the clearance of Q103-HTT via autophagy (FIGS. 2N and 20). Imaging assays based on analyzing IBs also confirmed the enhancing effect of CCT2 on protein aggregate clearance (FIGS. 6J and 6K).

To determine if CCT2 regulates the clearance of other aggregation-prone proteins, the inventors analyzed LC3 colocalization and turnover of Tau (P301L) and SOD1 (G93A). Similarly, CCT2 colocalized with Tau (P301L) aggregates and promoted LC3 recruitment to the aggregates. The inventors observed multiple puncta triple positive for Tau (P301L), CCT2, and LC3 (FIG. 4, arrows). In addition, the area of triple-positive puncta almost equaled to the increase of LC3-Tau (P301L) colocalization caused by CCT2 expression (FIGS. 4A and 4B). The data indicate that CCT2 directly promotes autophagosome incorporation of Tau (P301L). Consistently, CCT2 expression enhanced lysosome-dependent clearance of Tau (P301L) and SOD1 (G93A) (FIGS. 4C-4F).

To determine the specific effect of autophagy on aggregate clearance, the inventors removed soluble Q103-HTT using digitonin permeabilization of the plasma membrane. CCT2 knockdown largely compromised insoluble Q103-HTT degradation, which was restored by CCT2 re-expression (FIGS. 4G and 4H). Notably, the turnover of CCT2 was also inhibited by Bafilomycin Al or autophagy gene knockdown (FIG. 2N). In addition, CCT2 cofractionated with LC3-II and colocalized with autophagosome (FIGS. 2K, 4K, and 4A), and Bafilomycin Al treatment increased the amount of cellular and autophagosome-localized CCT2 (FIGS. 41 and 4J). Therefore the data, together with those showing the incorporation of CCT2 into the autophagosome (FIG. 2L), indicate that CCT2 is a substrate of autophagy, a character shared by autophagy receptors. In support of the notion, proteomic studies by other groups also detected CCT2 as a component of the autophagosome.

### CCT2 Binds to ATG8s Via Non-Classical LC3-Interaction Region Motifs

In co-immunoprecipitation (co-IP), CCT2 interacted with the six ATG8 family members with a preference for LC3C, in which the C-terminal one-third of CCT2 (D3), which corresponds to part of the equatorial domain, accounts for the association (FIGS. 5A and 5B). An involvement of LC3C in aggrephagy was also reported by another study.

Four of the five other chaperones (CCT1, CCT3, CCT6, and HSPA9, but not HSP90AB1) which promoted autophagosome association with IBs and lysosome-dependent polyQ-HTT turnover also associated with ATG8s with a preference for LC3C (FIGS. 6A-6E). HSPA9 is primarily a mitochondrial chaperone with multiple cellular localizations. Its long-form, likely the cytosolic form containing the transit peptide, associated with LC3C and IBs (FIG. 6E). CCT5 and CCT8, which had little effect on polyQ-HTT degradation, did not associate with LC3C (FIG. 6G). The data suggest a correlation of ATG8 association with involvement of aggrephagy among the chaperones tested.

Further mapping of the LC3C interaction region of CCT2-D3 using synthetic peptide pull-down found that a peptide (P7) covering aa 490-519 directly interacted with the purified LC3C (FIG. 5C). Interestingly, the inventors did not find a canonical LIR motif within P7. Instead, the inventors noticed that two triple consecutive hydrophobic residues (VLL and VIL) resemble the NDP52 CLIR-motif which is composed of LVV and accounts for LC3C interaction. Mutation of VLL and VIL abolished the binding of P7 to LC3C (FIG. 5C), indicating that these two triple-residue motifs contribute to LC3C interaction. Requirement of the motif for LC3C association was confirmed by co-IP analysis in which mutation of either VLL or VIL rendered CCT2 deficient of association with LC3C (FIG. 5D). In an in vitro pull-down assay, the WT CCT2 directly interacts with LC3C, which was abolished by mutation of VLL and VIL (FIG. 5E). The inventors term the VLL and VIL on P7 as VLIR-motif. The inventors also examined the association with GABARAP and GL1, two ATG8 family members which also strongly associate with CCT2 (FIG. 5A). Similarly, in peptide pull-down and co-IP, the two ATG8 family members directly interacted with P7 and associated with CCT2 in a VLIR-dependent manner (FIGS. 6H and 61). Therefore two VLIR motifs in CCT2 account for the interaction with LC3C, GABARAP, and GL1.

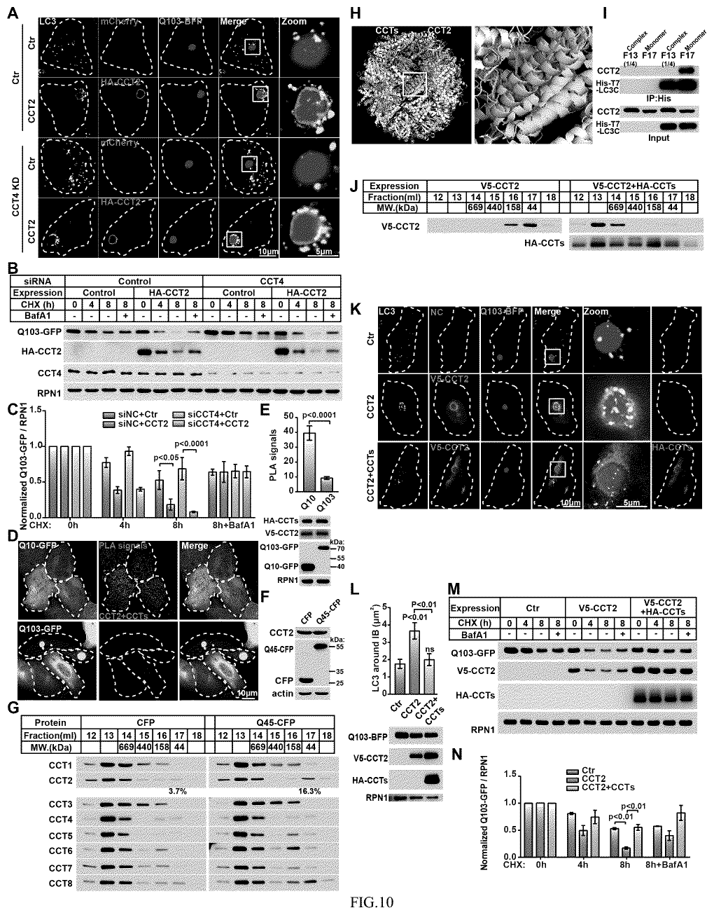

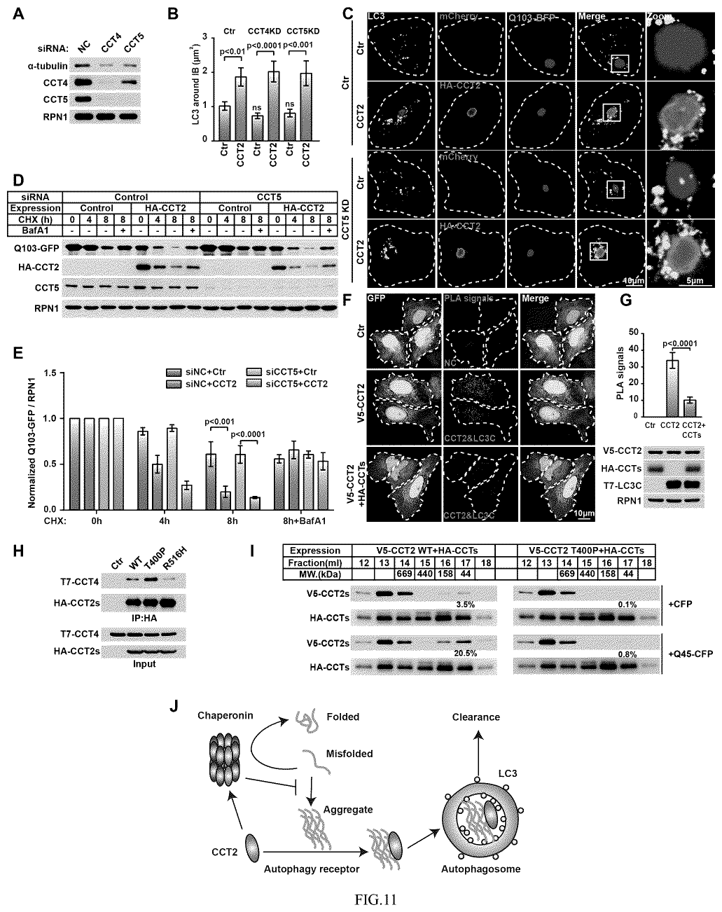

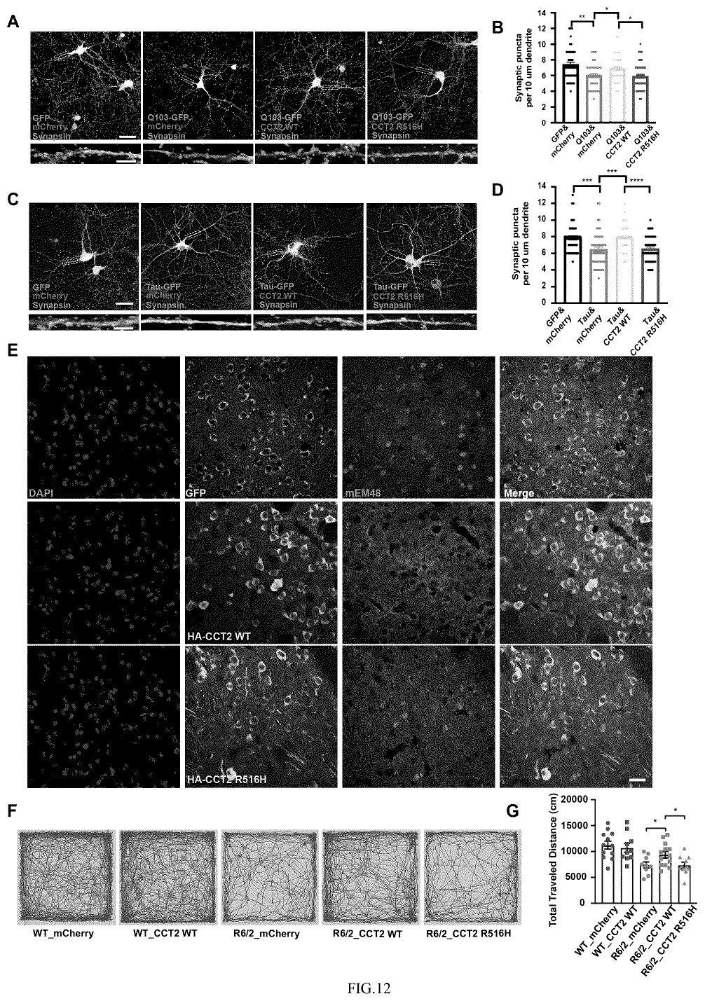

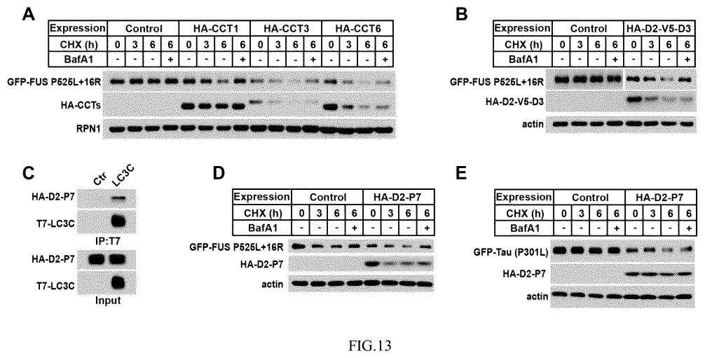

Noticeably, the double VLIR-motif mutant (mVL (I) L) of CCT2 failed to promote autophagic membrane association with IBs nor did it rescue the defect of digitonin insoluble Q103-HTT aggregate clearance caused by CCT2 depletion (FIGS. 5F-51). The dependence of VLIR on protein aggregate clearance was also confirmed by an imaging assay, in which CCT2 but not the VLIR mutant promoted the clearance of protein aggregates/IBs (FIGS. 6J and 6K). The functional loss of the VLIR mutant may not be due to the reduction of TRIC activity because the CCT2-VLIR mutant associated with CCT4 and restored the level of α-tubulin (an indicator of TRIC activity) equally well with the WT CCT2 in CCT2-depleted cells (FIGS. 6L and 6M). Therefore the data indicate that interacting with ATG8s is essential for CCT2 to promote autophagic membrane targeting and aggregate degradation.

Two CCT2 point mutations (T400P and R516H) were reported to cause Leber Congenital Amaurosis (LCA), a hereditary congenital retinopathy with severe macular degeneration. Although a moderate compromise of TRIC function was proposed, the two mutants were still able to largely restore the level of α-tubulin after CCT2-depletion compared with WT CCT2 (FIG. 6M). Instead, association with LC3C was dramatically decreased in the two mutants compared to WT CCT2 and consequently, the mutants were defective in promoting the recruitment of autophagic membrane to the IBs and Q103-HTT degradation (FIGS. 5J-50). The mutants also failed to be degraded via the lysosome compared to the WT CCT2 in the CHX chase assay, indicating that they lost the character of the autophagy receptor (FIG. 5N). The R516H localizes adjacent to the VIL motif (FIG. 5J). Therefore, it may affect VIL interaction with LC3C. How T400P affects LC3C association is explored below. Although pending further evidence, the data implies that deficiency of CCT2-mediated aggrephagy may be related to retinopathy.

CCT2 Associates with Aggregation-Prone Proteins but not Ubiquitin

CCT2 co-precipitated with the aggregation-prone proteins the turnover of which was regulated by CCT2 as shown above (FIGS. 7A-7C). This is consistent with the data that CCT2 associates with IBs of polyQ-HTT and the aggregates of Tau mutant protein (FIGS. 2A and 3A). The CCT2 apical domain is responsible for binding to polyQ-HTT (FIG. 7D), which echoes the previous finding that the apical domain of chaperonin subunits binds to misfolded/unfolded substrates. The data indicate that, as a chaperone protein, CCT2 interacts with aggregation-prone proteins via its intrinsic ability to bind misfolded/unfolded proteins during aggrephagy.

In contrast to P62, CCT2 did not co-precipitate with polyubiquitions synthesized in vitro or from the cell lysates (FIGS. 7E and 7F). In addition, CCT2 associated with polyQ-HTT in the IB irrespective of cargo ubiquitination, as the K-R mutant of polyQ-HTT showed a similar interaction signal with the WT counterpart in the Duolink PLA assay (FIGS. 7G and 7H). Consistently, CCT2 expression promoted the lysosome-dependent clearance of polyQ-HTT K-R mutant equally well with the WT counterpart (FIGS. 71-7K). Therefore, it is likely that CCT2 may associate with aggregation-prone proteins and promote their clearance independent of substrate ubiquitination.

### CCT2 Acts Independently of Known Pathways of Degrading Aggregation-Prone Proteins

To understand the relationship between CCT2 and these ubiquitin-binding receptors in aggrephagy, the inventors determined CCT2-LC3 association, autophagic membrane recruitment, and Q103-HTT autophagic degradation in cells triply depleted of P62, NBR1, and TAXIBP1. Deficiency of the three receptors did not affect CCT2-LC3C association, CCT2-promoted autophagic membrane recruitment to IBs, and CCT2-enhanced Q103-HTT clearance (FIGS. 6N and 8A-8D). The data indicate that CCT2 acts independent of the three ubiquitin-binding receptors in regulating aggrephagy.

CMA was also reported to regulate the clearance of soluble form of aggregation-prone proteins. Depletion of HSC70, the key chaperone receptor recognizing the KFERQ-motif of the cargoes, did not affect the association of CCT2 with LC3C (FIG. 6N). Nor did it compromise the CCT2-promoted autophagic membrane with IBs and lysosome-dependent clearance of Q103-HTT (FIGS. 8E-8H). Together, the data indicate that CCT2 acts independently of multiple ubiquitin-binding receptors and CMA.

CCT2 Promotes the Clearance of Protein Condensates with Little Liquidity

Liquid-liquid phase separation was shown as a transition stage before aggregation-prone proteins form solid protein aggregates. It has been proposed that selective autophagy preferentially clears protein condensates with certain amount of liquidity while solid aggregate is not a good substrate for aggrephagy. To determine the involvement of liquidity in CCT2-mediated clearance of protein condensates, the inventors employed an established FUS liquid-to-solid transition model to generate protein condensates with different states of liquidity (FIG. 9A). Via increasing the expression time of FUS with a disease mutation (P525L), the inventors observed protein condensates with decreasing liquidity from 24 to 72 h expression based on fluorescence recovery after photobleaching (FRAP) (FIGS. 9A and 9B). In the CHX chase experiment, the basal lysosome-dependent degradation of FUS (P525L) is reduced with the decrease of liquidity (FIGS. 9C-9H, Compare 24, 48 and 72 h, lane 1-4 of the IBs). This is consistent with the notion that aggregates with decreased liquidity compromise degradation via aggrephagy. Triple KD of P62/NBR1/TAXIBP1 but not CCT2 compromised the basal lysosome-dependent turnover of liquid FUS (P525L) (24 h), indicating that the ubiquitin-binding receptors are primarily involved in the autophagic degradation of liquid FUS (P525L) condensates (FIGS. 9K and 9L). Interestingly, expression of CCT2, instead of the VLIR mutant, enhanced the clearance of FUS (P525L) with low liquidity compared to those of high liquidity (FIGS. 9C-9H, Compare 24, 48 and 72 h, lane 1-4 vs lane 5-8 vs lane 9-12). Especially, CCT2 exerted the most enhancing effect of clearance on FUS (P525L) with 72 h expression in which liquidity was barely detected based on FRAP indicating the likelihood of solid state (FIGS. 9A, 9B, 9G, and 9H).

Cation-interactions mediated by arginine and tyrosine were shown to regulate liquid-to-solid transition of FUS, and arginine methylation is an important tune of the process. The FUS mutants with 16 amino acids mutated to arginine (P525L+16R) were reported to have increased liquid-to-solid transition. The inventors employed this mutant to further confirm the reverse correlation between liquidity and CCT2-promoted clearance. Consistent with the previous study, the FUS (P525L+16R) was expressed with decreased liquidity compared to FUS (P525L) in which fluorescence recovery was barely observed (likely to be a solid state) for the FUS (P525L+16R) after 48 h expression together with reduced lysosome-dependent clearance compared to FUS (P525L) of 48 h expression (FIGS. 9A, 9B, 9E, 9F, 9I, and 9J). Again, compared to FUS (P525L), the FUS (P525L+16R) clearance was more efficiently promoted by CCT2 but not the VLIR mutant (FIGS. 9E, 9F, 9I and 9J).

It has been shown that chaperones regulate the phase transition of aggregation-prone proteins. However, expression of CCT2 did not affect the liquidity of FUS (P525L) or FUS (P525L+16R) condensates suggesting that CCT2 did not promote their clearance via altering liquid-to-solid transition (FIGS. 9A and 9B). Instead, CCT2 preferentially increased the amount of FUS (P525L) and FUS (P525L+16R) with little liquidity in the autophagosome (FIGS. 8Q and 8R). The data together with the requirement of VLIR motif in promoting FUS (P525L) and FUS (P525L+16R) clearance (FIGS. 9C-9J) indicate that CCT2 mediates degradation of the protein condensates via aggrephagy.

Different from CCT2, expression of NBR1 or TAXIBP1 enhanced the clearance of FUS (P525L) condensates with liquidity but not the solid aggregate FUS (P525L+16R) (FIGS. 9M-9P). The data, together with the KD experiments which showed the requirement of P62, NBR1 or TAXIBP1 but not CCT2 in lysosome-dependent clearance of liquid FUS (P525L) (FIGS. 9K and 9L), indicate that CCT2 and the ubiquitin-binding receptors respectively degrade protein condensates with different liquidity. The ubiquitin-binding receptors select cargoes with liquidity, whereas CCT2 prefers those with little liquidity in aggrephagy.

To explore why CCT2 preferentially enhances the clearance of FUS condensates with little liquidity, the inventors produced granules of liquid-liquid phase separation and solid aggregates of FUS (P525L) using a previous approach (FIG. 9T). In a protein recruitment assay, the CCT2 protein preferentially associated with solid aggregates compared to liquid granules in vitro (FIG. 9U). Therefore, the data suggest that CCT2 interacts with protein condensates with little liquidity and promotes their autophagic clearance.

### CCT2 Functions Independent of the Chaperonin TRIC in Aggrephagy

It has been shown that the proper function of TRIC requires all subunits. In the TRIC, CCT4 and CCT5 are two neighbors of CCT2. To determine the involvement of TRIC complex formation in CCT2-regulated aggrephagy, the inventors depleted CCT4 and CCT5 respectively to disrupt the TRIC complex. The compromise of TRIC function was confirmed by a reduction of α-tubulin after CCT4 or CCT5 RNAi (FIG. 11A). CCT2 expression increased autophagic membrane association with IBs similarly in control, CCT4 KD, and CCT5 KD cells (FIGS. 10A, 11B and 11C). Consistently, CCT2 expression promoted lysosome-dependent Q103-HTT degradation in control and CCT4 or CCT5 KD cells (FIGS. 10B, 10C, 11D and 11E). The data indicate that CCT2 regulates aggrephagy independent of the integrity of the TRIC complex.

To determine the status of CCT2 in mediating aggrephagy, the inventors analyzed the association between CCT2 and TRIC subunits in the absence and presence of Q103-HTT using a Duolink PLA assay. Interestingly, Q103-HTT expression inhibited the association between CCT2 and TRIC subunits, suggesting that accumulation of the aggregation-prone protein affects partition of CCT2 in the TRIC (FIGS. 10D and 10E). To confirm, the inventors incubated cell lysates, in which majority of CCT2 existed in the form of TRIC complex, with Q45-CFP (1.6 μM) with a comparable concentration of Q103-HTT in the cytosol in our experiments (1.5-2.0 μM, data not shown) (FIG. 10F). In gel-filtration, Q45-CFP protein increased CCT2 in the ˜44KD monomer fraction by ˜3.5 fold compared to CFP (FIG. 10G, from 3.7% to 16.3% of total CCT2 in the monomer fraction). Several other CCT subunits also appeared to increase in the non-TRIC complex fractions, suggesting partial disruption of TRIC by polyQ-HTT (FIG. 10G). Together the data indicate that accumulation of polyQ-HTT promotes the monomer form of CCT2 and may affect the integrity of the TRIC complex.

The VLIR motif locates in the equatorial domain of CCT2 and is buried into the TRIC complex (FIG. 10H). It is likely that via dissociating from the TRIC complex, the VLIR motif is exposed and able to associate with ATG8s. The notion is confirmed by a pull-down experiment in which CCT2 in the monomer instead of in the TRIC complex fraction interacted with LC3C (FIG. 101). In addition, the exogenously expressed CCT2 which promoted aggrephagy were primarily monomeric (FIG. 10J). Co-expression of other TRIC subunits decreased monomeric CCT2 by forming the TRIC complex and therefore compromised the CCT2 association with LC3C (FIGS. 11F and 11G), as well as the CCT2 promoted autophagic membrane recruitment to IBs and Q103-HTT degradation (FIGS. 10J-10N). The inventors also found that in addition to CCT2, the rest of the TRIC subunits were able to associate with IBs (FIG. 10K), which is consistent with previous studies showing that the TRIC complex and subunits localize to protein aggregates. Importantly, the T400P mutation enhanced CCT2 association with CCT4 and partition into the TRIC complex (FIG. 11H). In addition, the mutation compromised the release of CCT2 (T400P) from the TRIC complex caused by polyQ-HTT (FIG. 11I), and therefore decreased the ability of CCT2 to associate with LC3 and to promote aggrephagy as shown above.

Together the data indicate a scenario of CCT2 dissociation from the TRIC complex induced by excessive aggregation-prone proteins as a switch of chaperonin function from protein folding to autophagy. The monomeric CCT2 is able to associate with ATG8s and therefore act as an autophagy receptor to promote the degradation of protein aggregates (FIG. 11J).

### Expression of CCT2 Relieves Neurodegeneration Phenotype

Expression of WT CCT2 but not the aggrephagy-deficient R516H mutant restored neuron synapse loss caused by Q103-HTT or Tau (P301L) expression in primary culture (FIGS. 12A-12D). In addition, AAV-mediated CCT2 expression in mouse striatum reduced Q140 inclusion body in the knockin model described above, whereas the ATG8 binding-deficient CCT2 R516H had no effect (FIG. 12E). In an open field assessment, CCT2 WT expression instead of the CCT2 R516H conferred significant total distance traveled in R6/2 transgenic mouse model of Huntington's disease (FIGS. 12F and 12G). Together, the data indicate that expression of CCT2 clears aggregation-prone proteins and improves the performance of neurodegenerative mice.

### CCT1/3/6 and CCT2 Fusion Proteins Promote Clearance of Solid Aggregates

The inventors also determined the function of CCT1/3/6 in the clearance of solid aggregates. Expression of CCT1/3/6 accelerated the degradation of FUS P525L+16R (FIG. 13A), suggesting these chaperonin subunits also function as autophagy receptors in clearance of solid aggregates.

To modify CCT2 for more effective application, the inventors fused the functional domains of CCT2, the D2 which associates with protein aggregates and the D3 which interacts with LC3, with a V5 (SEQ ID NO:17) as a linker between the two domains. Expression of the D2-V5-D3 accelerated the autophagic clearance of FUS P525L+16R (FIG. 13B), indicating that the D2 and D3 fusion protein is enough for autophagy receptor function of CCT2. The inventors further optimized the CCT2 by fusing the D2 and P7 peptide of D3. D2-P7 also associated with LC3C and effectively accelerated the degradation of FUS P525L+16R and the Tau (P301L) (FIGS. 13 C, D and E). Therefore, the modified D2-V5-D3 or D2-P7 have good application prospects in aggregation related diseases.

### GKPIPNPLLGLDST (SEQ ID NO:17).

It will be apparent to those skilled in the art that variations and modifications of the present invention may be made without departing from the scope or spirit of the present invention. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for promoting clearance of solid protein aggregates and/or aggregation-prone proteins, or promoting ATG8 targeting to inclusion bodies comprising:
giving reagent, which is used to at least one of the following:
overexpress chaperones or enhance the activity of chaperones;
enhance the chaperones interaction with ATG8s;
promote the disassociation of TRiC to produce free subunits;
overexpress/apply the D2 and/or D3 domain of CCT2 or enhance the D2 and/or D3 domain activity of CCT2;
overexpress/apply the P7 Peptide of CCT2 or enhance the P7 Peptide activity of CCT2;
enhance the activity of amino acids 503 ˜505 and/or 513 ˜515 of CCT2;
overexpress/apply the peptide or enhance the peptide activity, wherein the peptide comprises amino acids 503 to 515 of CCT2 and optionally at least 10 amino acids upstream of amino acid 503 or at least 10 amino acids downstream of amino acid 515.

2. The method of claim 1, wherein the chaperone comprises at least one of the following: CCT2, CCT6, CCT1, CCT3, HSPA9 and HSP90AB1.

3. The method of claim 1, wherein the free subunits comprise at least one of the following: CCT2, CCT6, CCT1, CCT3.

4. The method of claim 1, wherein the reagent comprises expression vector with chaperones coding nucleic acid or compounds, protein or factors used for enhancing the activity of chaperones;
optionally, wherein the reagent comprises expression vector with D2 and/or D3 domain coding nucleic acid or compounds, protein or factors used for enhancing the activity of D2 and/or D3 domain.

5. The method of claim 1, wherein the CCT2 coding nucleic acid has the nucleotide sequence shown in SEQ ID No: 1; or
CCT6 coding nucleic acid has the nucleotide sequence shown in SEQ ID No: 2; or
CCT1 coding nucleic acid has the nucleotide sequence shown in SEQ ID No: 3; or
CCT3 coding nucleic acid has the nucleotide sequence shown in SEQ ID No: 4; or
HSPA9 coding nucleic acid has the nucleotide sequence shown in SEQ ID No: 5; or
HSP90AB1 coding nucleic acid has the nucleotide sequence shown in SEQ ID No: 6.

6. The method of claim 4, wherein the expression vector is AAV.

7. The method of claim 1, wherein the method is independent of cargo ubiquitination.

8. The method of claim 1, wherein the method is realized through autophagy.

9. The method of claim 1, wherein the activity of chaperones is the ability of chaperones to degrade solid protein aggregates and/or aggregation-prone proteins by autophagy.

10. A method for treating or preventing of diseases caused by protein aggregation comprising:
administration medication to subjects, wherein the medication is used for at least one of the following:
overexpressing chaperones or enhancing the activity of chaperones;
enhancing the chaperones interaction with solid protein aggregates and/or aggregation-prone proteins;
enhancing the chaperones interaction with ATG8s;
promoting the disassociation of TRIC to produce free subunits;
overexpressing/applying the D2 and/or D3 domain of CCT2 or enhancing the D2 and/or D3 domain activity of CCT2;
overexpressing/applying the P7 Peptide of CCT2 or enhancing the P7 Peptide activity of CCT2;
enhancing the activity of amino acids 503 ˜505 and/or 513 ˜515 of CCT2;
overexpressing/applying the peptide or enhancing the peptide activity, wherein the peptide comprises amino acids 503 to 515 of CCT2 and optionally at least 10 amino acids upstream of amino acid 503 or at least 10 amino acids downstream of amino acid 515.

11. The method of claim 10, wherein the administration is by injection.

12. The method of claim 11, wherein the injection is in situ or intravenous administration.

13. The method of claim 10, wherein the diseases caused by protein aggregation including at least one of the following:
neurodegenerative diseases, eye disease, type II diabetes, and amyloid transthyretin cardiomyopathy;
optionally, the neurodegenerative diseases include at least one of the following:
Alzheimer's disease (AD), Parkinson's disease (PD), Huntington's disease (HD), amyotrophic lateral sclerosis (ALS), dementia with Lewy bodies, frontotemporal dementia, different types of spinocerebellar ataxia (SCA), pick disease.

14. A method for screening drugs for treatment or prevention diseases caused by protein aggregation comprising:
contact the model with the drug to be screened, and compare the changes of at least one of the following before and after contact in the model;
the expression quantity of chaperones or the activity of chaperones;
the binding force of chaperones with ATG8s;
the binding force of chaperones with solid protein aggregates and/or aggregation-prone proteins;
the quantity of TRIC free subunits;
the expression quantity of the D2 and/or D3 domain of CCT2 or the activity of the D2 and/or D3 domain of CCT2;
the expression quantity of the P7 Peptide of CCT2 or the activity of P7 Peptide of CCT2;
the activity of amino acids 503 ˜505 and/or 513 ˜515 of CCT2;
the expression quantity of the peptide or the activity of the peptide, wherein the peptide comprises amino acids 503 to 515 of CCT2 and optionally at least 10 amino acids upstream of amino acid 503 or at least 10 amino acids downstream of amino acid 515;
and
based on the change, determine whether the drug to be screened is the target drug.

15. The method of claim 14, wherein after exposure compared with before exposure, a rise in at least one of the following:
the expression quantity of chaperones or the activity of chaperones;
the binding force of chaperones with ATG8s;
the binding force of chaperones with solid protein aggregates and/or aggregation-prone proteins;
the quantity of TRiC free subunits;
the expression quantity of the D2 and/or D3 domain of CCT2 or the activity of the D2 and/or D3 domain of CCT2;
the expression quantity of the P7 Peptide of CCT2 or the activity of P7 Peptide of CCT2;
the activity of amino acids 503 ˜505 and/or 513 ˜515 of CCT2;
the expression quantity of the peptide or the activity of the peptide, wherein the peptide comprises amino acids 503 to 515 of CCT2 and optionally at least 10 amino acids upstream of amino acid 503 or at least 10 amino acids downstream of amino acid 515;
is an indication that the drug to be screened is the target drug.

16. The method of claim 14, wherein the chaperone comprises at least one of the following:
CCT2, CCT6, CCT1, CCT3, HSPA9 and HSP90AB1.

17. The method of claim 14, wherein the model is cultured cell lines, nerve cell, tissue or mice
optionally, the model is CCT2 knockdown or overexpression cultured cell lines, tissue or mice.

18. The method of claim 17, wherein the cultured cell lines, nerve cell or tissue has solid protein aggregates and/or aggregation-prone proteins.

19. The method of claim 14, wherein the diseases caused by protein aggregation including at least one of the following:
neurodegenerative diseases, eye disease, type II diabetes, and amyloid transthyretin cardiomyopathy;
optionally, the neurodegenerative diseases include at least one of the following:
Alzheimer's disease (AD), Parkinson's disease (PD), Huntington's disease (HD), amyotrophic lateral sclerosis (ALS), dementia with Lewy bodies, frontotemporal dementia, different types of spinocerebellar ataxia (SCA), pick disease.
